(19) United States
(12) Patent Application Publication
(10) Pub. No.: US 2005/0124537 A1
(43) Pub. Date: Jun. 9, 2005

(54) Modulators of receptors for parathyroid hormone and parathyroid hormone-related protein

(75) Inventors: Paul Kostenuik, Newbury Park, CA (US); Colin V. Gegg, Newbury Park, CA (US); Mark Anthony Jarosinski, Boulder, CO (US); Olaf Boris Kinstler, Newbury Park, CA (US)

Correspondence Address: U. S. Patent Operations/ TJG;Dept. 4300, M/S 27-4-A, AMGEN, INC., One Amgen Center Drive, Thousand Oaks, CA, 91320-1799, US

(73) Assignee: Amgen Inc.

(21) Appl. No.: 09/999,608

(22) Filed: Oct. 31, 2001

Related U.S. Application Data
(63) Continuation-in-part of application No. 09/843,221, filed on Apr. 26, 2001, now Pat. No. 6,756,480.
(60) Provisional application No. 60/266,673, filed on Feb. 6, 2001.
(60) Provisional application No. 60/214,860, filed on Jun. 28, 2000.
(60) Provisional application No. 60/200,053, filed on Apr. 27, 2000.

(51) Int. Cl. C07K 14/635; A61K 38/29

(52) U.S. Cl. 514/12; 530/399

(57) ABSTRACT

The present invention concerns therapeutic agents that modulate the activity of PTH and PTHrP. In accordance with the present invention, modulators of PTH and PTHrP comprise: (a) a PTH/PTHrP modulating domain; and (b) a vehicle, such as a polymer (e.g., PEG or dextran) or an Fc domain, which is preferred; wherein the vehicle is covalently attached to the C-terminus of the PTH/PTHrP modulating domain or through a sidechain at any residue from residue 14 through the C-terminal residue. The vehicle and the PTH/PTHrP modulating domain may be linked through the N- or C-terminus of the PTH/PTHrP modulating domain, as described further below. The preferred vehicle is PEG. Preferred PTH/PTHrP modulating domains comprise the PTH and PTHrP-derived amino acid sequences described hereinafter. Other PTH/PTHrP modulating domains can be generated by phage display, RNA-peptide screening and the other techniques mentioned herein. Such peptides typically will be modulators of both PTH activity and PTHrP activity, although such techniques can be used to generate peptide sequences that serve as selective modulators (e.g., agonists of PTH activity but not PTHrP activity).

### CROSS-REFERENCE TO RELATED APPLICATIONS

This application is a continuation-in-part of U.S. Ser. No. 09/843,221, filed Apr. 26, 2001, which claims the benefit of U.S. Provisional Application No. 60/266,673, filed Feb. 6, 2001, U.S. Provisional Application No. 60/214,860, filed Jun. 28, 2000,and U.S. Provisional Application No. 60/200,053, filed Apr. 27, 2000, which are hereby incorporated by reference.

### FIELD OF THE INVENTION

This invention relates to parathyroid hormone PTH), parathyroid hormone-related protein (PTHrP) and modulators of PTH and PTHrP receptors. This invention also relates to proteins modified for extended half-life and, in particular, to proteins modified with polyethylene glycol.

### BACKGROUND OF THE INVENTION

PTH and PTHrP play important physiological roles in calcium homeostasis and in development, respectively. Calcium concentration in the blood is tightly regulated, due to the essential role of calcium in cell metabolism. PTH is an endocrine hormone which is secreted from the parathyroid gland in response to decreased serum calcium levels. PTH acts directly to increase bone resorption and to stimulate renal calcium reabsorption, thus increasing or preserving circulating calcium stores. PTH also indirectly increases calcium absorption in the gut by stimulating the renal hydroxylation of vitamin D.

Both primary and secondary hyperparathyroidism are conditions that are associated with excessive levels of circulating parathyroid hormone. Through the aforementioned pathways, excess PTH levels can cause hypercalcemia and osteopenia. Bone resorption inhibitors such as bisphosphonates and OPG can effectively protect bone and can inhibit the skeleton's contribution to hypercalcemia. However, the calcemic effects of hyperparathyroidism on the kidney and gut are not addressed by currently available therapy.

PTHrP is produced by many cell types, and plays an important role in regulating skeletal development. Postnatally, the roles for PTHrP are less clearly defined. Circulating levels of PTHrP are essentially non-detectable in normal healthy adults. However, many tumors of diverse embryological origins produce and secrete PTHrP in quantities sufficient to cause hypercalcemia. In fact, humoral hypercalcemia of malignancy (HHM) is the most common paraneoplastic syndrome, which accounts for significant patient morbidity and mortality.

Currently, HHM is treated with saline hydration followed by bone resorption inhibitors such as bisphosphonates. This treatment regimen typically takes 3-4 days to achieve significant reductions in serum calcium, and the effects are relatively short-lived (less than one month). For patients with high circulating levels of PTHrP, the effects of current treatment options are even less impressive. Repeated administration of conventional therapies are usually progressively less effective. These limitations to current therapy strongly indicate an unmet medical need for rapid, effective, and long-lasting treatments for HHM.

A major reason for the limited benefits of current HHM therapy is the failure to directly inhibit PTHrP, which is very well established as the principal pathophysiologic factor in HHM. Bone resorption inhibitors such as bisphosphonates only inhibit bone resorption, while PTHrP also has significant calcemic effects on the kidney and the gut. Total neutralization of PTHrP would be the ideal adjuvant therapeutic approach to treatment of HHM.

Both PTH and PTHrP interact with PTH-1 receptor, which accounts for most of their known effects. Mannstadt et al. (1999), Am. J. Physiol. 277. 5Pt 2. F665-75 (1999). Only PTH interacts with the newly discovered PTH-2 receptor. Id. PTHrP can be changed to a PTH-2 receptor agonist, however, by changing two residues to the residues at those positions in PTH. Gardella et al. (1996), J. Biol. Chem. 271 (33): 19888-93.

An N-terminal fragment of PTH has been used as a therapeutic agent. Intermittently administered native PTH-(1-84) exhibits osteogenic properties, and it has been recognized for decades that these properties can be fully realized with the C-terminally truncated fragment PTH-(1-34). Both peptides bind and activate the PTH-1 receptor with similar affinities, causing the activation of adenylate cyclase (AC) as well as phospholipase C (PLC). AC activation through PTH-1 receptor generates CAMP, while PLC activation through PTH-1 receptor generates PKC and intracellular calcium transients. PTH-(1-34) can maximally activate both the AC and the PLC pathways. It has been demonstrated that the anabolic effects of PTH-(1-34) require short intermittent (daily) exposures Dobnig (1998), Endocrinol. 138: 4607-12. In human trials on postmenopausal women, daily subcutaneous injection of low doses of PTH(1-34) were shown to result in impressive bone formation in the spine and femoral neck with significant reduction in incidence of vertebral fractures. These clinical data reveal PTH as one of the most efficacious agents tested for osteoporosis.

Truncated PTH fragments have diminished AC/cAMP activation and similarly diminished anabolic activity. Rixon et al. (1994), J. Bone Min. Res. 9: 1179-89; Hilliker et al. (1996), Bone 19: 469-477; Lane et al. (1996), J. Bone Min. Res. 11: 614-25. Such truncated PTH fragments have this diminished activity(Rixon et al. (1994); Hilliker et al. (1996); Lane et al. (1996)) even if they maintain full agonism towards PKC. Rixon et al., (1994). These observations have led to the proposal that the AC/cAMP pathway is critical for the bone anabolic properties of PTH, while the PLC/PKC pathway is dispensable in this regard. Rixon et al., (1994); Whitfield et al. (1996), Calcified Tissue International 53: 81-7.

An opposing, but not mutually exclusive, theory suggests that PLC activation (in addition to AC) might also be an important property of anabolic PTH fragments. Takasu (1998), Endocrinol. 139: 4293-9. The apparent absence of PLC activation by some anabolic C-terminally truncated PTH peptides may be an artifact of insensitive assay methods combined with lower receptor binding. Takasu (1998). Progressive truncations from the C-terminus of PTH-(1-34) result in stepwise reductions in binding affinity for the PTH1R Takasu (1998). PKC activation through PTH-1 receptor appears to be acutely sensitive to binding affinity and to receptor density (Guo et al. (1995), Endocrinol 136: 3884-91), whereas cAMP activation is far less sensitive to these variables. As such, hPTH-(1-31) has a slightly reduced (1-6 fold) affinity for PTH-1 receptor compared to hPTH-(1-34), while hPTH-(1-30) has a significantly reduced (10-100 fold) affinity Takasu (1998). Perhaps due to this decreased PTH-1 receptor affinity, PTH-(1-30) is a weak and incomplete agonist for PLC activation via the rat PTH-1 receptor.

Compared to PTH-(1-34), PTH-(1-31) has similar or slightly reduced anabolic potential (Rixon et al. (1994); whitfield et al. (1996), Calcified Tissue International 53: 81-7; Whitfield et al. (1996), Calcified Tissue International 65: 143-7), binding affinity for PTH1R, and cAMP induction (Takasu (1998)). PTH-(1-31) also has slightly reduced PLC activation. Takasu (1998). In healthy humans, infusion of PTH-(1-31) and PTH-(1-34) had similar stimulatory effects on plasma and urinary cAMP concentration, but unlike PTH-(1-34), PTH-(1-31) failed to elevate serum calcium, plasma 1,25(OH)2D3, or urinary N-TX levels. Fraher et al. (1999), J. Clin. Endocrin. Met. 84: 2739-43. These data suggest that PTH-(1-31) has diminished capacity to induce bone resorption and to stimulation vitamin D synthesis, which is a favorable profile for bone anabolic agents.

PTH-(1-30) was initially shown to lack anabolic properties Whitfield et al. (1996), Calcified Tissue International 53: 81-7. More recently, however, it has been demonstrated that PTH-(1-30) is anabolic when administered at very high doses (400-2,000 μg/kg, vs. 80 μg/kg for PTH-(1-34)). The lower potency of PTH-(1-30) could be predicted by its lower binding affinity for PTH-1 receptor, its diminished cAMP activation, and/or to its greatly diminished PKC activation. Takasu (1998). It remains to be determined whether PTH-(1-30) has a similar or even more desirable reduction in apparent bone resorption activity.

PTH-(1-28) is the smallest reported fragment to fully activate cAMP. Neugebauer et al. (1995), Biochem. 34: 8835-42. However, hPTH-(1-28) was initially reported to have no osteogenic effects in OVX rats.

Miller et al. (1997), J. Bone Min. Res. 12: S320 (Abstract). Recently, a very high dose of PTH-(1-28) (1,000 μg/kg/day) was shown to be anabolic in OVX rats, whereas 200 μg/kg/day was ineffective. Whitfield et al. (2000), J. Bone Min. Res. 15: 964-70. The diminished or absent anabolic effects of some truncated PTH fragments has been attributed to rapid clearance in vivo. Rixon et al. (1994).

Recombinant and modified proteins are an emerging class of therapeutic agents. Useful modifications of protein therapeutic agents include combination with the “Fc” domain of an antibody and linkage to polymers such as polyethylene glycol (PEG) and dextran. Such modifications are discussed in detail in a patent application entitled, “Modified Peptides as Therapeutic Agents,” U.S. Ser. No. 09/428,082, WO 00/24782, which is hereby incorporated by reference in its entirety.

A much different approach to development of therapeutic agents is peptide library screening. The interaction of a protein ligand with its receptor often takes place at a relatively large interface. However, as demonstrated for human growth hormone and its receptor, only a few key residues at the interface contribute to most of the binding energy. Clackson et al. (1995), Science 267: 383-6. The bulk of the protein ligand merely displays the binding epitopes in the right topology or serves functions unrelated to binding. Thus, molecules of only “peptide” length (2 to 40 amino acids) can bind to the receptor protein of a given large protein ligand. Such peptides may mimic the bioactivity of the large protein ligand (“peptide agonists”) or, through competitive binding, inhibit the bioactivity of the large protein ligand (“peptide antagonists”).

Phage display peptide libraries have emerged as a powerful method in identifying such peptide agonists and antagonists. See, for example, Scott et al. (1990), Science 249: 386; Devlin et al. (1990), Science 249: 404; U.S. Pat. No. 5,223,409, issued Jun. 29, 1993; U.S. Pat. No. 5,733,731, issued Mar. 31, 1998; U.S. Pat. No. 5,498,530, issued Mar. 12, 1996; U.S. Pat. No. 5,432,018, issued Jul. 11, 1995; U.S. Pat. No. 5,338,665, issued Aug. 16, 1994; U.S. Pat. No. 5,922,545, issued Jul. 13, 1999; WO 96/40987, published Dec. 19, 1996; and WO 98/15833, published Apr. 16, 1998 (each of which is incorporated by reference in its entirety). In such libraries, random peptide sequences are displayed by fusion with coat proteins of filamentous phage. Typically, the displayed peptides are affinity-eluted against an antibody-immobilized extracellular domain of a receptor. The retained phages may be enriched by successive rounds of affinity purification and repropagation. The best binding peptides may be sequenced to identify key residues within one or more structurally related families of peptides. See, e.g., Cwirla et al. (1997), Science 276: 1696-9, in which two distinct families were identified. The peptide sequences may also suggest which residues may be safely replaced by alanine scanning or by mutagenesis at the DNA level. Mutagenesis libraries may be created and screened to further optimize the sequence of the best binders. Lowman (1997), Ann. Rev. Biophys. Biomol. Struct. 26: 401-24.

Structural analysis of protein-protein interaction may also be used to suggest peptides that mimic the binding activity of large protein ligands. In such an analysis, the crystal structure may suggest the identity and relative orientation of critical residues of the large protein ligand, from which a peptide may be designed. See, e.g., Takasaki et al. (1997), Nature Biotech. 15: 1266-70. These analytical methods may also be used to investigate the interaction between a receptor protein and peptides selected by phage display, which may suggest further modification of the peptides to increase binding affinity.

Other methods compete with phage display in peptide research. A peptide library can be fused to the carboxyl terminus of the lac repressor and expressed in E. coli. Another E. coli-based method allows display on the cell's outer membrane by fusion with a peptidoglycan-associated lipoprotein (PAL). Hereinafter, these and related methods are collectively referred to as “E. coli display.” In another method, translation of random RNA is halted prior to ribosome release, resulting in a library of polypeptides with their associated RNA still attached. Hereinafter, this and related methods are collectively referred to as “ribosome display.” Other methods employ peptides linked to RNA; for example, PROfusion technology, Phylos, Inc. See, for example, Roberts & Szostak (1997), Proc. Natl. Acad. Sci. USA, 94:12297-303. Hereinafter, this and related methods are collectively referred to as “RNA-peptide screening.” Chemically derived peptide libraries have been developed in which peptides are immobilized on stable, non-biological materials, such as polyethylene rods or solvent-permeable resins. Another chemically derived peptide library uses photolithography to scan peptides immobilized on glass slides. Hereinafter, these and related methods are collectively referred to as “chemical-peptide screening.” Chemical-peptide screening may be advantageous in that it allows use of D-amino acids and other unnatural analogues, as well as non-peptide elements. Both biological and chemical methods are reviewed in Wells & Lowman (1992), Curr. Opin. Biotechnol. 3: 355-62. Conceptually, one may discover peptide mimetics of any protein using phage display, RNA-peptide screening, and the other methods mentioned above.

### SUMMARY OF THE INVENTION

The present invention concerns therapeutic agents that modulate the activity of PTH and PTHrP. In accordance with the present invention, modulators of PTH and PTHrP comprise:
a) a PTH/PTHrP modulating domain, preferably the amino acid sequence of PTH/PTHrP modulating domains of PTH and/or PTHrP, or sequences derived therefrom by phage display, RNA-peptide screening, or the other techniques mentioned above; and
b) a vehicle, such as a polymer (e.g., PEG or dextran) or an Fc domain, which is preferred;
   wherein the vehicle is covalently attached to the carboxyl terminus of the PTH/PTHrP modulating domain. The preferred vehicle is an Fc domain, and the preferred Fc domain is an IgG Fc domain. Preferred PTH/PTHrP modulating domains comprise the PTH and PTHrP-derived amino acid sequences described hereinafter. Other PTH/PTHrP modulating domains can be generated by phage display, RNA-peptide screening and the other techniques mentioned herein. Such peptides typically will be antagonists of both PTH and PTHrP, although such techniques can be used to generate peptide sequences that serve as selective inhibitors (e.g., inhibitors of PTH but not PTHrP).

Further in accordance with the present invention is a process for making PTH and PTHrP modulators, which comprises:
a) selecting at least one peptide that binds to the PTH-1 or PTH-2 receptor; and
b) covalently linking said peptide to a vehicle.
   The preferred vehicle is an Fc domain. Step (a) is preferably carried out by selection from the peptide sequences in Tables 1A, 1B, and 2 hereinafter or from phage display, RNA-peptide screening, or the other techniques mentioned herein.

The compounds of this invention may be prepared by standard synthetic methods, recombinant DNA techniques, or any other methods of preparing peptides and fusion proteins. Compounds of this invention that encompass non-peptide portions may be synthesized by standard organic chemistry reactions, in addition to standard peptide chemistry reactions when applicable.

The primary use contemplated for the compounds of this invention is as therapeutic or prophylactic agents. The vehicle-linked peptide may have activity comparable to-or even greater than-the natural ligand mimicked by the peptide.

The compounds of this invention may be used for therapeutic or prophylactic purposes by formulating them with appropriate pharmaceutical carrier materials and administering an effective amount to a patient, such as a human (or other mammal) in need thereof. Other related aspects are also included in the instant invention.

Of particular interest in the present invention are molecules comprising PTH/PTHRP modulating domains having a shortened PTH C-terminal sequence, such as PTH-(1-28) or (1-34). The prior art shows no anabolic studies using sustained duration delivery of such C-terminally truncated PTH fragments. Although the art does not suggest it, molecules comprising smaller fragments such as PTH-(1-30)-Fc can be anabolic on their own. Despite their weak agonism towards PLC (see Background of the Invention), hPTH-(1-30) is nearly as effective at cAMP stimulation as is hPTH-(1-34). While not wanting to be constrained by theory, the inventors note that the anabolic properties of PTH fragments may be selectively related to their cAMP activation, rather than PLC activation, so that PTH fragments with reduced receptor affinity will have a favorable anabolic profile. It is possible that continuous exposure to truncated PTH fragments would have a different, and more favorable effect on bone compared to continuous exposure to PTH-(1-34) or PTH-(1-84) that has been demonstrated in humans by Fraher et al. (1999).

Numerous additional aspects and advantages of the present invention will become apparent upon consideration of the figures and detailed description of the invention.

### BRIEF DESCRIPTION OF THE FIGURES

FIG. 1 shows exemplary Fc dimers that may be derived from an IgG1 antibody. “Fc” in the figure represents any of the Fc variants within the meaning of “Fc domain” herein. “X¹” and “X²” represent peptides or linker-peptide combinations as defined hereinafter. The specific dimers are as follows:

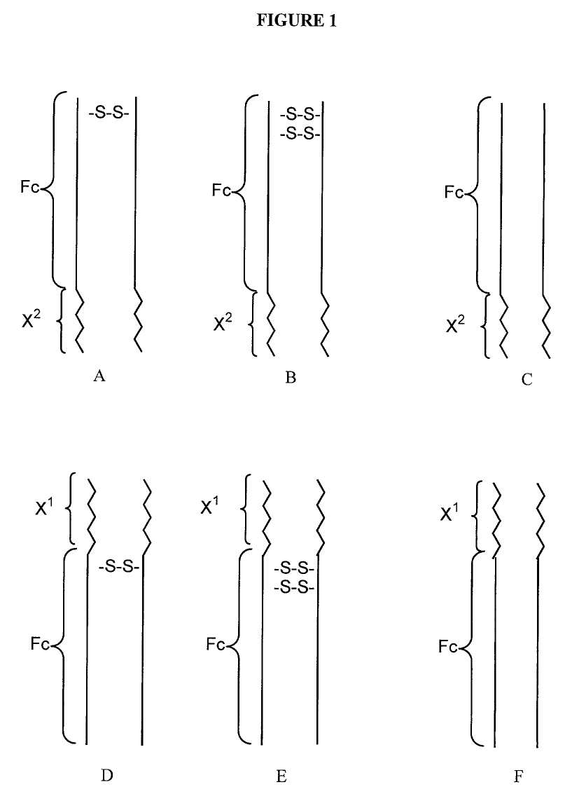

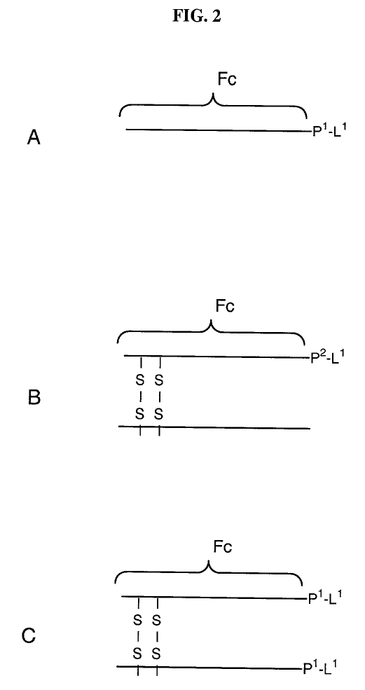

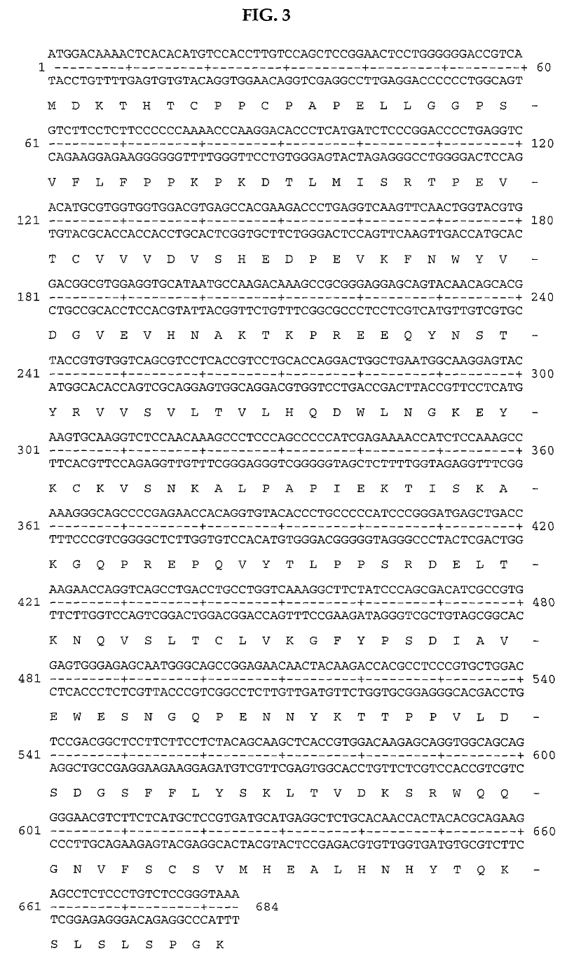

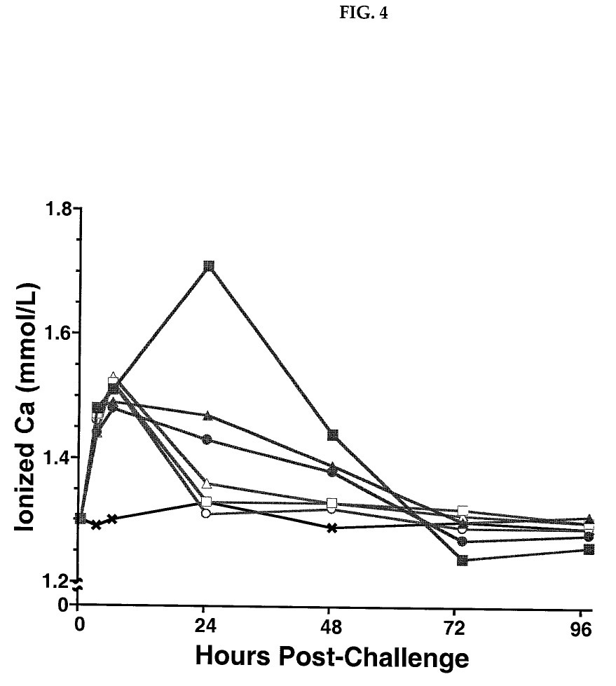

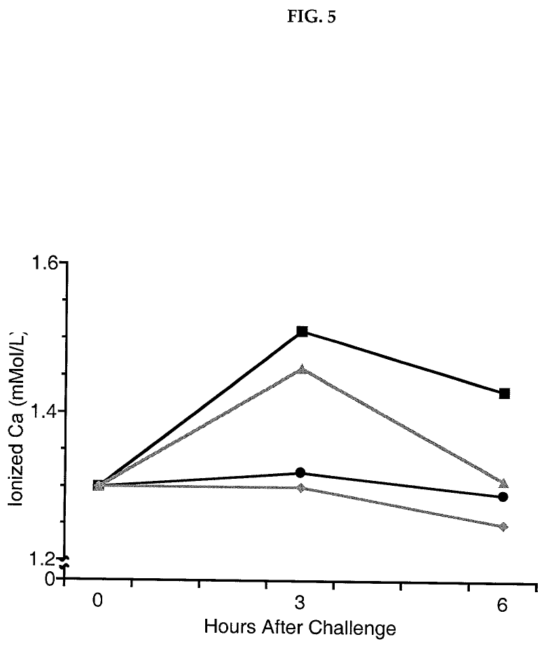

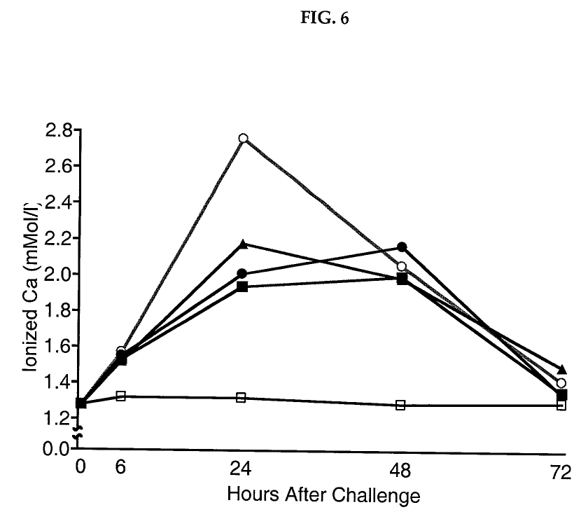

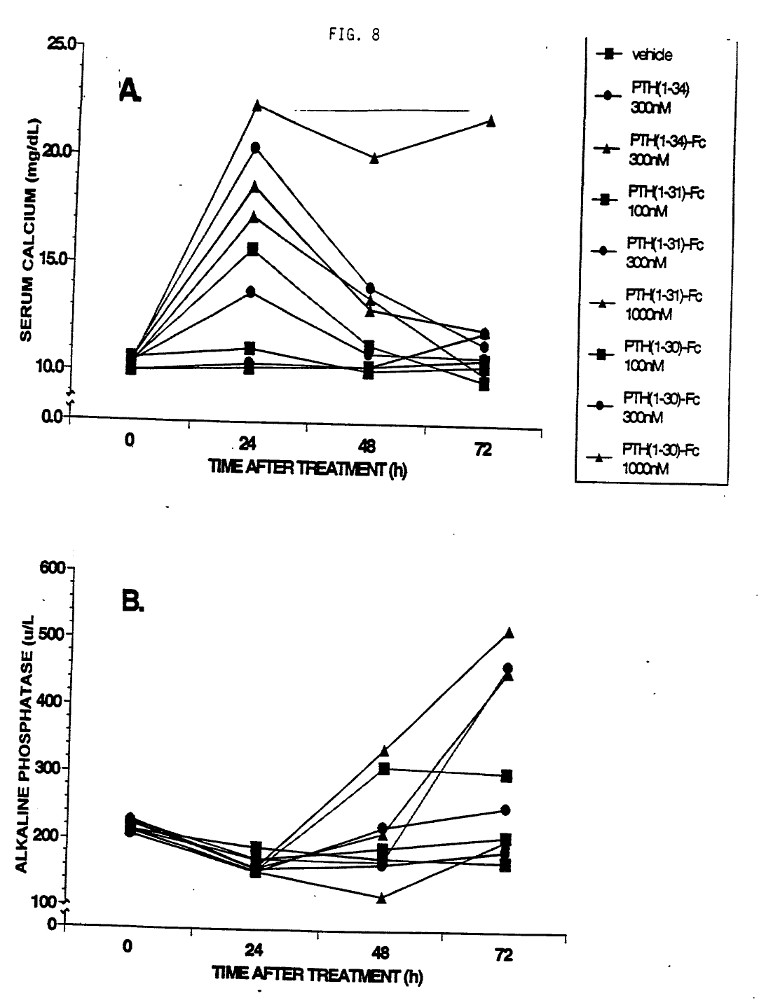

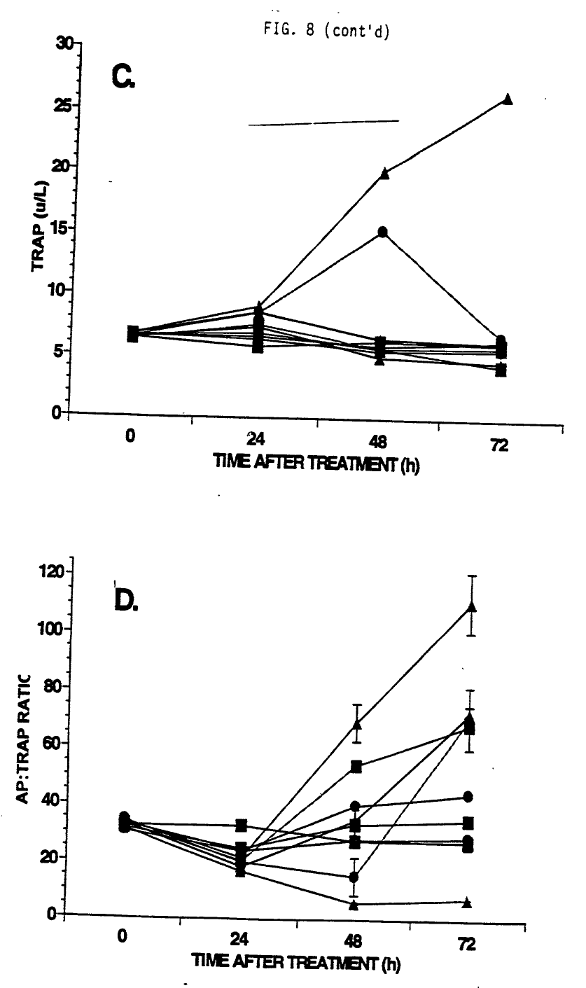

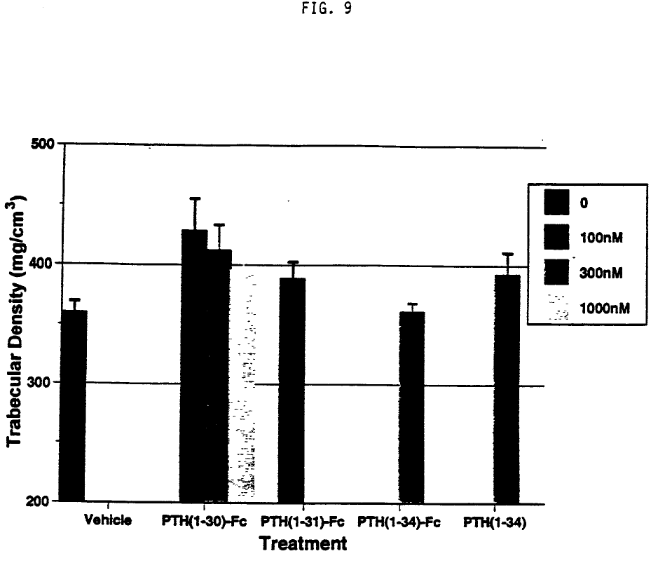

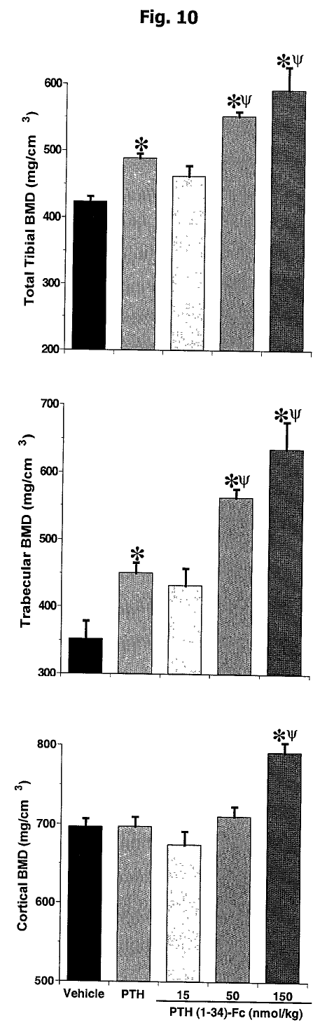

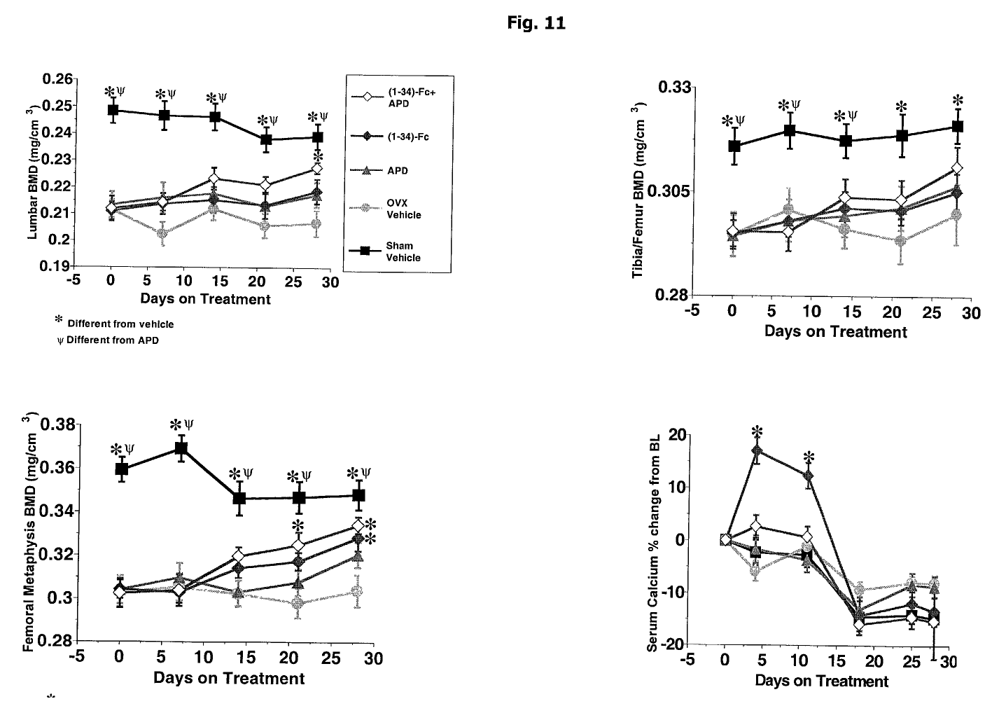

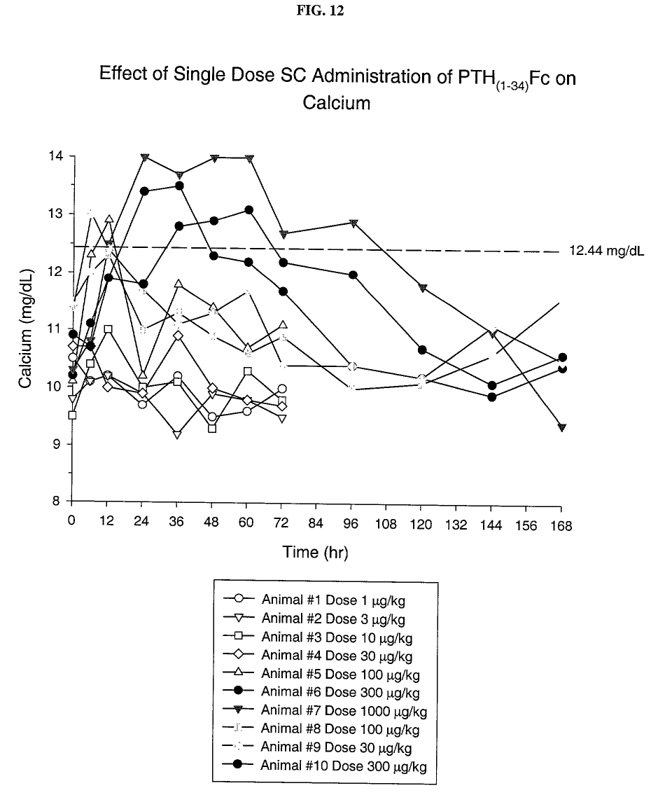

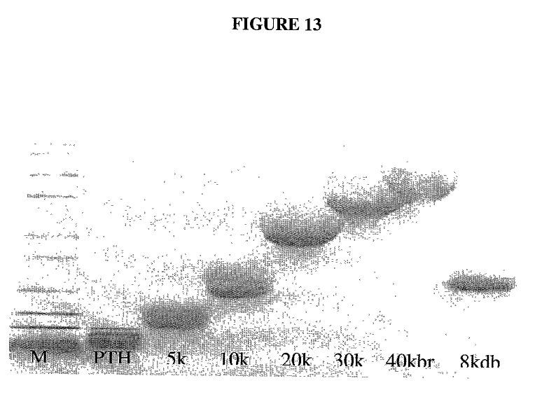

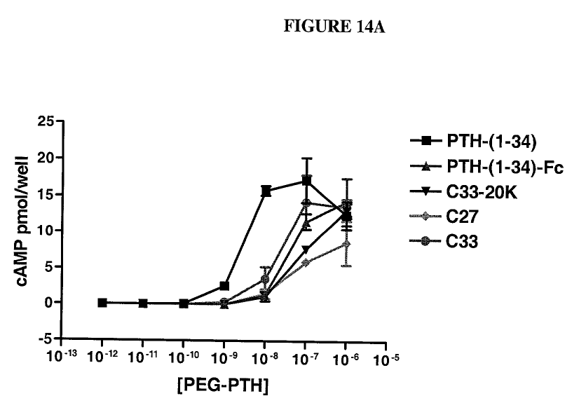

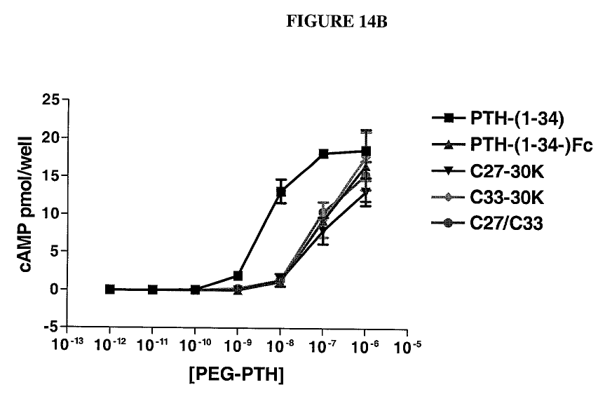

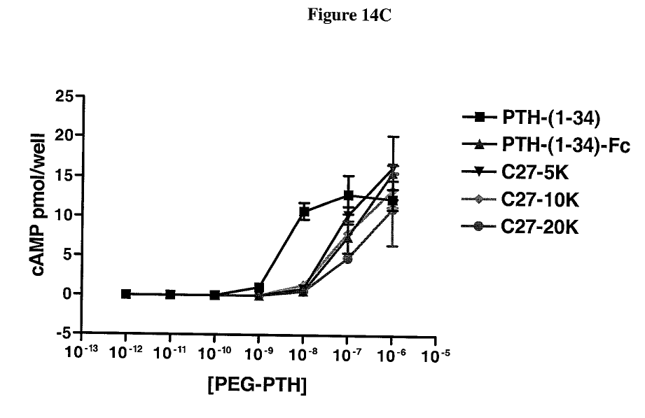

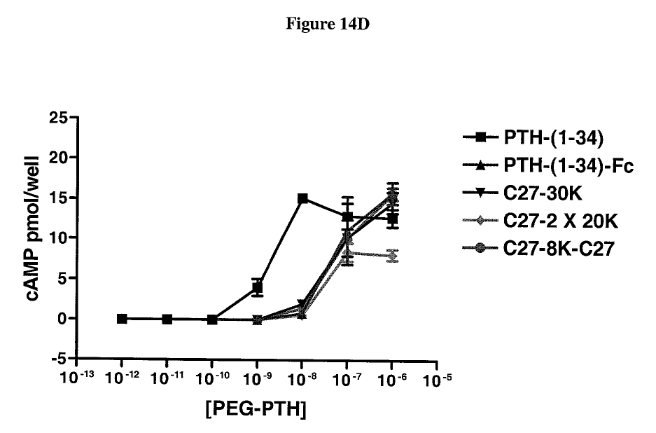

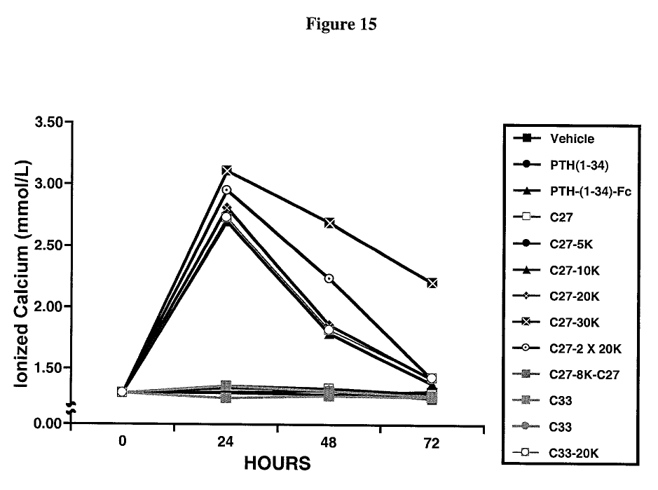

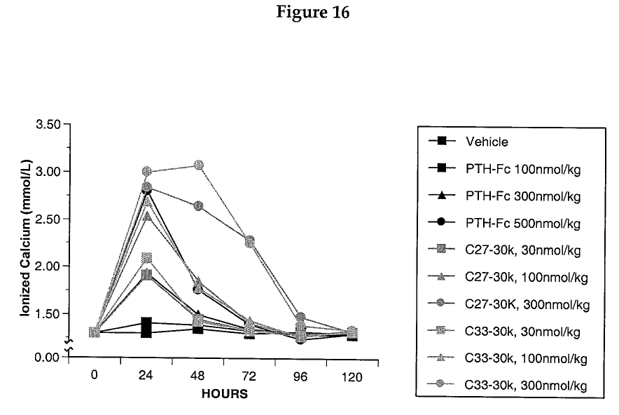

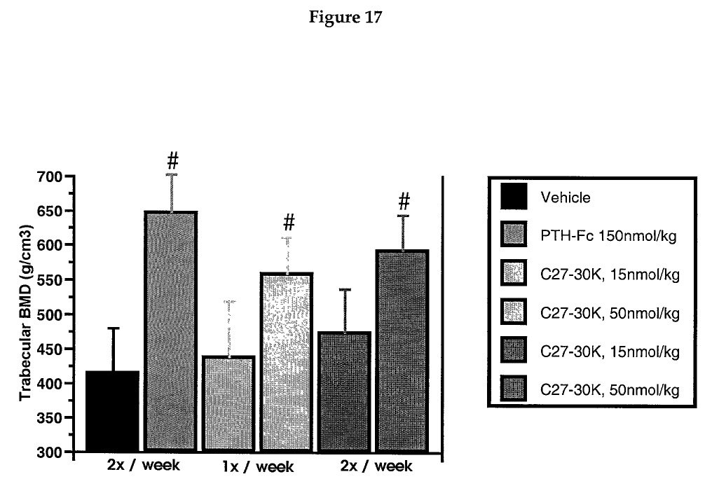

A: Single disulfide-bonded dimers. IgG1 antibodies typically have two disulfide bonds at the hinge region between the constant and variable domains. The Fc domain in FIG. 1A may be formed by truncation between the two disulfide bond sites or by substitution of a cysteinyl residue with an unreactive residue (e.g., alanyl). In FIG. 1A, the Fc domain is linked at the C-terminus of the peptide.

B: Doubly disulfide-bonded dimers. This Fc domain may be formed by truncation of the parent antibody to retain both cysteinyl residues in the Fc domain chains or by expression from a construct including a sequence encoding such an Fc domain. In FIG. 1B, the Fc domain is linked at the C- terminus of the peptide.

C: Noncovalent dimers. This Fc domain may be formed by elimination of the cysteinyl residues by either truncation or substitution. One may desire to eliminate the cysteinyl residues to avoid impurities formed by reaction of the cysteinyl residue with cysteinyl residues of other proteins present in the host cell. The noncovalent bonding of the Fc domains is sufficient to hold together the dimer.

Other dimers may be formed by using Fc domains derived from different types of antibodies (e.g., IgG2, IgM).

FIG. 2 shows the structure of additional compounds of the invention. FIG. 2A shows a single chain molecule and may also represent the DNA construct for the molecule. FIG. 2B shows a dimer in which the linker-peptide portion is present on only one chain of the dimer. FIG. 2C shows a dimer having the peptide portion on both chains. The dimer of FIG. 2C will form spontaneously in certain host cells upon expression of a DNA construct encoding the single chain as shown in FIG. 3. In other host cells, the cells could be placed in conditions favoring formation of dimers or the dimers can be formed in vitro.

FIG. 3 shows exemplary nucleic acid and amino acid sequences (SEQ ID NOS: 1 and 2, respectively) of human IgG1 Fc that may be used in this invention.

FIG. 4 shows the calcemic response of normal mice to PTH-(1-34) and to PTH-(1-34)-Fc. Mice were challenged with vehicle (PBS, —X—), or with PTH-(1-34) (open symbols) or with PTH-(1-34)-Fc (closed symbols). Doses were 156 nmol/kg (circles), 469 nmol/kg (triangles) or 1,560 nmol/kg (squares). Data represent group means, n=6 mice/group.

FIG. 5 shows that [Asn10,Leu11]PTHrP-(7-34)-Fc inhibits the calcemic response of normal mice to PTHrP. Normal male mice were injected SC with vehicle (PBS, circles) or with human PTHrP-(1-34) at 0.5 mg/kg (squares). PTHrP-challenged mice were then immediately injected SC with [Asn10,Leu11]PTHrP-(7-34)-Fc at 10 mg/kg (triangles) or 30 mg/kg (diamonds). Data represent group means, with an n of 6 mice/group.

FIG. 6 shows the effect of [Asn10,Leu11]PTHrP-(7-34)-Fc on chronic hypercalcemia induced by PTH-(1-34)-Fc. Normal male mice were challenged once by SC injection with PTH-(1-34)-Fc (30 mg/kg) (open circles), or with vehicle (PBS, open squares). Some PTH-(1-34)-Fc-challenged mice were treated once, at the time of challenge, with [Asn10,Leu11]PTHrP-(7-34)-Fc at 10 (closed triangle), 30 (closed circle), or 100 mg/kg (closed square). All doses of [Asn10,Leu11]PTHrP-(7-34)-Fc caused a significant suppression of PTH-(1-34)-Fc-mediated hypercalcemia. Data represent means ±SEM, n=5 mice/group.

FIG. 7 shows CAMP accumulation in ROS 17/2.8 rat osteoblast-like cells. Cultures were treated with the phosphodiesterase inhibitor IBMX and then challenged for 15 minutes with various PTH fragments. CAMP was measured by ELISA.

FIG. 8 shows the effects of single treatments on clinical chemistry. Peripheral blood was obtained daily for 3 days following single subcutaneous injections of the indicated compounds. FIG. 8A shows total serum calcium; FIG. 8B, alkaline phosphatase (AP), a marker of osteoblast activity; FIG. 8C, tartrate-resistant acid phosphatase (TRAP), a marker of osteoclast activity, and FIG. 8D, AP:TRAP ratio, an index of relative osteoblas: osteoclast activity.

FIG. 9 shows the effects of PTH constructs on bone mineral density. Peripheral quantitative computed tomography (pQCT) was performed on the proximal tibial metaphysis of mice on day 15, after injections of PTH constructs on day 0, 5 and 10.

FIG. 10 shows the effect of twice-weekly PTH-(1-34)-Fc versus daily PTH-(1-34) on tibial, trabecular, and cortical bone mineral density (BMD). Daily PTH [PTH-(1-34)] was given at 80 μg/kg/day (20 nmol/kg/day).

FIG. 11 shows the effects of twice-weekly treatment on BMD and serum calcium in aged ovariectomized (OVX) rats. Eleven months after OVX, rats were treated twice per week with phosphate-buffered saline (PBS, vehicle) or with APD (0.5 mg/kg) or with PTH-(1-34)-Fc (50 nmol/kg). DEXA was performed weekly. Blood was drawn 24 hours after the second weekly injection, when the calcemic effects of PTH-Fc are typically maximal.

FIG. 12 shows the effect of a single subcutaneous injection of PTH-(1-34)-Fc into OVX cynomologus monkeys. Monkeys were injected with PTH-(1-34)-Fc at doses of 1-30 μg/kg (n=1/group) or 100-1000 μg/kg (n=2/group). Serum was analyzed for total calcium. The dotted line indicates the threshold for hypercalcemia, based on an elevation of calcium greater than three standard deviations above the normal mean, on two or more consecutive timepoints.

FIG. 13 shows SDS-PAGE analysis of representative samples of purified PEG-PTH (1-34) conjugate prepared from cysteine 27 PTH (1-34) analog and 5 kD, 10 kD, 20 kD and 30 kD linear PEG polymers, a 40 kD branched polymer and the 8 kD bis-functional polymer respectively.

FIGS. 14A through 14D show CAMP response of murine MC3T3-E1 osteoblast cultures to treatment with various PTH constructs. Cultures were incubated with test materials for 10 minutes at room temperature. Cell lysates were analyzed for cAMP production by ELISA.

FIG. 15 shows the hypercalcemic response of young male BDF1 mice to various PTH constructs. All treatments were single subcutaneous injections of 300 nmol/kg (PTH referent).

FIG. 16 shows the hypercalcemic response of young male BDF1 mice to a single subcutaneous injection of PTH-(1-34)-Fc or PEGPTH constructs. Blood ionized calcium was measured at the timepoints indicated in the figure.

FIG. 17 shows the effect of PTH-(1-34)-Fc or C27-30K PEG-PTH on tibial bone mineral density (BMD) in adult male BDF1 mice. Mice were treated by subcutaneous. injection either once or twice per week for 4 weeks. The symbol # indicates significant difference from vehicle (PBS)-treated mice, by two-way ANOVA.

### DETAILED DESCRIPTION OF THE INVENTION

Definition of Terms

The terms used throughout this specification are defined as follows, unless otherwise limited in specific instances.

The term “comprising” means that a compound may include additional amino acids on either or both of the N- or C-termini of the given sequence. Of course, these additional amino acids should not significantly interfere with the activity of the compound.

The term “acidic residue” refers to amino acid residues in D- or L-form having sidechains comprising acidic groups. Exemplary acidic residues include D and E.

The term “aromatic residue” refers to amino acid residues in D- or L-form having sidechains comprising aromatic groups. Exemplary aromatic residues include F, Y, and W.

The term “basic residue” refers to amino acid residues in D- or L-form having sidechains comprising basic groups. Exemplary basic residues include H, K, and R.

The terms “hydrophilic residue” and “Haa” refer to amino acid residues in D- or L-form having sidechains comprising at least one hydrophilic functional group or polar group. Exemplary hydrophilic residues include C, D, E, H, K, N, Q, R, S, and T.

The terms “lipophilic residue” and “Laa” refer to amino acid residues in D- or L-form having sidechains comprising uncharged, aliphatic or aromatic groups. Exemplary lipophilic sidechains include F, I, L, M, V, W, and Y. Alanine (A) is amphiphilic—it is capable of acting as a hydrophilic or lipophilic residue. Alanine, therefore, is included within the definition of both “lipophilic residue” and “hydrophilic residue.”

The term “nonfunctional residue” refers to amino acid residues in D- or L-form having sidechains that lack acidic, basic, or aromatic groups. Exemplary nonfunctional amino acid residues include M, G, A, V, I, L and norleucine (Nle).

The term “vehicle” refers to a molecule that prevents degradation and/or increases half-life, reduces toxicity, reduces immunogenicity, or increases biological activity of a therapeutic protein. Exemplary vehicles include an Fc domain (which is preferred) as well as a linear polymer (e.g., polyethylene glycol (PEG), polylysine, dextran, etc.); a branched-chain polymer (see, for example, U.S. Pat. No. 4,289,872 to Denkenwalter et al., issued Sep. 15, 1981; 5,229,490 to Tam, issued Jul. 20, 1993; WO 93/21259 by Frechet et al., published 28 Oct. 1993); a lipid; a cholesterol group (such as a steroid); a carbohydrate or oligosaccharide (e.g., dextran); human serum albumin (HSA) and related molecules; transtheratin (TTR) and related molecules; or any natural or synthetic protein, polypeptide or peptide that binds to a salvage receptor. Vehicles are further described hereinafter.

The term “native Fc” refers to molecule or sequence comprising the sequence of a non-antigen-binding fragment resulting from digestion of whole antibody, whether in monomeric or multimeric form. The original immunoglobulin source of the native Fc is preferably of human origin and may be any of the immunoglobulins, although IgG1 and IgG2 are preferred. Native Fc's are made up of monomeric polypeptides that may be linked into dimeric or multimeric forms by covalent (i.e., disulfide bonds) and non-covalent association. The number of intermolecular disulfide bonds between monomeric subunits of native Fc molecules ranges from 1 to 4 depending on class (e.g., IgG, IgA, IgE) or subclass (e.g., IgG1, IgG2, IgG3, IgA1, IgGA2). One example of a native Fc is a disulfide-bonded dimer resulting from papain digestion of an IgG (see Ellison et al. (1982), Nucleic Acids Res. 10: 4071-9). The term “native Fc” as used herein is generic to the monomeric, dimeric, and multimeric forms.

The term “Fc variant” refers to a molecule or sequence that is modified from a native Fc but still comprises a binding site for the salvage receptor, FcRn. International applications WO 97/34631 (published 25 September 1997) and WO 96/32478 describe exemplary Fc variants, as well as interaction with the salvage receptor, and are hereby incorporated by reference in their entirety. Thus, the term “Fc variant” comprises a molecule or sequence that is humanized from a non-human native Fc. Furthermore, a native Fc comprises sites that may be removed because they provide structural features or biological activity that are not required for the fusion molecules of the present invention. Thus, the term “Fc variant” comprises a molecule or sequence that lacks one or more native Fc sites or residues that affect or are involved in (1) disulfide bond formation, (2) incompatibility with a selected host cell (3) N-terminal heterogeneity upon expression in a selected host cell, (4) glycosylation, (5) interaction with complement, (6) binding to an Fc receptor other than a salvage receptor, or (7) antibody-dependent cellular cytotoxicity (ADCC). Fc variants are described in further detail hereinafter.

The term “Fc domain” encompasses native Fc and Fc variant molecules and sequences as defined above. As with Fc variants and native Fc's, the term “Fc domain” includes molecules in monomeric or multimeric form, whether digested from whole antibody or produced by other means.

The term “multimer” as applied to Fc domains or molecules comprising Fc domains refers to molecules having two or more polypeptide chains associated covalently, noncovalently, or by both covalent and non-covalent interactions. IgG molecules typically form dimers; IgM, pentamers; IgD, dimers; and IgA, monomers, dimers, trimers, or tetramers. Multimers may be formed by exploiting the sequence and resulting activity of the native Ig source of the Fc or by derivatizing (as defined below) such a native Fc.

The term “dimer” as applied to Fc domains or molecules comprising Fc domains refers to molecules having two polypeptide chains associated covalently or non-covalently. Thus, exemplary dimers within the scope of this invention are as shown in FIGS. 1 and 2.

The terms “derivatizing” and “derivative” or “derivatized” comprise processes and resulting compounds respectively in which (1) the compound has a cyclic portion; for example, cross-linking between cysteinyl residues within the compound; (2) the compound is cross-linked or has a cross-linking site; for example, the compound has a cysteinyl residue and thus forms cross-linked dimers in culture or in vivo; (3) one or more peptidyl linkage is replaced by a non-peptidyl linkage; (4) the N-terminus is replaced by —NRR¹, NRC(O)R¹, —NRC(O)OR¹, —NRS(O)₂R¹, —NHC(O)NHR, a succinimide group, or substituted or unsubstituted benzyloxycarbonyl-NH—, wherein R and R¹ and the ring substituents are as defined hereinafter; (5) the C-terminus is replaced by —C(O)R² or —NR³R⁴ wherein R², R³ and R⁴ are as defined hereinafter; and (6) compounds in which individual amino acid moieties are modified through treatment with agents capable of reacting with selected side chains or terminal residues. Derivatives are further described hereinafter.

The term “peptide” refers to molecules of 1 to 85 amino acids, with molecules of 5 to 34 amino acids preferred. Exemplary peptides may comprise the PTH/PTHrP modulating domain of a naturally occurring molecule or comprise randomized sequences.

The term “randomized” as used to refer to peptide sequences refers to fully random sequences (e.g., selected by phage display methods or RNA-peptide screening) and sequences in which one or more residues of a naturally occurring molecule is replaced by an amino acid residue not appearing in that position in the naturally occurring molecule. Exemplary methods for identifying peptide sequences include phage display, E. coli display, ribosome display, RNA-peptide screening, chemical screening, and the like.

The term “PTH/PTHrP modulating domain” refers to any amino acid sequence that binds to the PTH-1 receptor and/or the PTH-2 receptor and comprises naturally occurring sequences or randomized sequences. Exemplary PTH/PTHrP modulating domains can be identified or derived as described in the references listed for Tables 1A and 2, which are hereby incorporated by reference in their entirety.

The term “PTH agonist” refers to a molecule that binds to PTH-1 or PTH-2 receptor and increases or decreases one or more PTH activity assay parameters as does full-length native human parathyroid hormone. An exemplary PTH activity assay is disclosed in Example 1.

The term “PTH antagonist” refers to a molecule that binds to PTH-1 or PTH-2 receptor and blocks or prevents the normal effect on those parameters by full length native human parathyroid hormone. An exemplary PTH activity assay is disclosed in Example 2.

The term “bone resorption inhibitor” refers to such molecules as determined by the assays of Examples 4 and 11 of WO 97/23614:, which is hereby incorporated by reference in its entirety. Exemplary bone resorption inhibitors include OPG and OPG-L antibody, which are described in WO 97/23614 and WO 98/46751, respectively, which are hereby incorporated by reference in their entirety.

Additionally, physiologically acceptable salts of the compounds of this invention are also encompassed herein. By “physiologically acceptable salts” is meant any salts that are known or later discovered to be pharmaceutically acceptable. Some specific examples are: acetate; trifluoroacetate; hydrohalides, such as hydrochloride and hydrobromide; sulfate; citrate; tartrate; glycolate; and oxalate.

Structure of Compounds

In General.

Exemplary PTH and PTHrP receptor binding amino acid sequences are described in Tables 1A, 1B, and 2. Other information on PTH and PTHrP is found in Mannstadt et al. (1999), Am. J. Physiol. 277. 5Pt 2: F665-75; and Gardella (1996), J. Biol. Chem. 271 (33): 19888-93. Each of these references is hereby incorporated by reference in its entirety.

From the foregoing sequences, the present inventors identified in particular preferred sequences derived from PTH and PTHrP. These sequences can be randomized through the techniques mentioned above by which one or more amino acids may be changed while maintaining or even improving the binding affinity of the peptide.

In the compositions of matter prepared in accordance with this invention, the peptide may be attached to the vehicle through the peptide's C-terminus. Thus, the vehicle-peptide molecules of this invention may be described by the following formula I:
P¹-(L¹)ₐ-F¹ I
and multimers thereof, wherein:

F¹ is a vehicle (preferably a PEG molecule) and is attached at the C-terminus of P¹-(L¹)ₐ or through a sidechain at any residue from residue 14 through the C-terminal residue;

P¹ is a sequence of a PTH/PTHrP modulating domain;

L¹ is a linker; and

a is 0 or 1.

Peptides.

Any number of peptides may be used in conjunction with the present invention. Peptides may comprise part of the sequence of naturally occurring proteins, may be randomized sequences derived from the sequence of the naturally occurring proteins, or may be wholly randomized sequences. Phage display and RNA-peptide screening, in particular, are useful in generating peptides for use in the present invention.

A PTH/PTHrP modulating domain sequence particularly of interest is of the formula II

| | |
|---|---|
| X^{N}X⁸HX¹⁰X¹¹X¹²KX¹⁴X¹⁵X¹⁶X¹⁷X¹⁸X¹⁹RX²¹X²²X²³X²⁴X²⁵X²⁶X²⁷X²⁸X^{C} | (SEQ ID NO: 3) |

wherein:

X^{N} is absent or is X³X⁴X⁵X⁶X⁷, X²X³X⁴X⁵X⁶X⁷, X¹X²X³X⁴X⁵X⁶X⁷, or YX¹X²X³X⁴X⁵X⁶X⁷;

X¹ is an amino acid residue (nonfunctional, hydrophilic or aromatic residue preferred; A, S or Y preferred);

X² is an amino acid residue (nonfunctional residue preferred, V most preferred);

X³ is an amino acid residue (hydrophilic residue preferred, S most preferred);

X⁴ is an amino acid residue (acidic residue preferred, E most preferred);

X⁵ is an amino acid residue (nonfunctional or basic residue preferred, H or I most preferred);

X⁶ is an amino acid residue (acidic or hydrophilic residue preferred, Q or E most preferred);

X⁷ is an amino acid residue (nonfunctional or aromatic residue preferred, L or F most preferred);

X⁸ is an amino acid residue (nonfunctional residue preferred, M or Nle most preferred);

X¹⁰ is an amino acid residue (an acidic or hydrophilic residue preferred, N or D most preferred);

X¹¹ is an amino acid residue (nonfunctional or basic residue preferred, L, R, or K most preferred);

X¹² is an amino acid residue (nonfunctional or aromatic residue preferred, G, F, or W most preferred);

X¹⁴ is an amino acid residue (basic or hydrophilic residue preferred, H or S most preferred);

X¹⁵ is an amino acid residue (nonfunctional residue preferred, with L or I most preferred);

X¹⁶ is an amino acid residue (nonfunctional or hydrophilic residue preferred, Q, N, S, or A most preferred);

X¹⁷ is an amino acid residue (acidic, hydrophilic, or nonfunctional residue preferred; S, D, or L most preferred);

X¹⁸ is an amino acid residue (nonfunctional residue preferred, M, L, V or Nle most preferred);

X¹⁹ is an amino acid residue (acidic or basic residue preferred, E or R most preferred);

X²¹ is an amino acid residue (nonfunctional residue or basic residue preferred; V, M, R, or Nle most preferred);

X²² is an amino acid residue (hydrophilic, acidic, or aromatic residue preferred, E or F most preferred);

X²³ is an aromatic or lipophilic residue (W or F preferred);

X²⁴ is a lipophilic residue (L preferred);

X²⁵ is an amino acid residue (hydrophilic or basic residue preferred, R or H most preferred);

X²⁶ is an amino acid residue (hydrophilic or basic residue preferred, K or H most preferred);

X²⁷ is an amino acid residue (lipophilic, basic, or nonfunctional residue preferred, K or L most preferred);

X²⁸ is an amino acid residue (lipophilic or nonfunctional residue preferred, L or I most preferred);

X^{C} is absent or is X²⁹, X²⁹X³⁰, X²⁹X³⁰X³¹, X²⁹X³⁰X³¹X³², X²⁹X³⁰X³¹X³²X³³, X²⁹X³⁰X³¹X³², X³³X³⁴, X²⁹X³⁰X³¹X³²X³³X³⁴X³⁵, or X²⁹X³⁰X³¹X³²X³³X³⁴X³⁵X³⁶;

X²⁹ is an amino acid residue (hydrophilic or nonfunctional residue preferred, Q or A most preferred);

X³⁰ is an amino acid residue (hydrophilic or acidic residue preferred, D or E most preferred);

X³¹ is an amino acid residue (lipophilic or nonfunctional residue preferred, V or I most preferred);

X³² is an amino acid residue (basic residue preferred, H most preferred);

X³³ is an amino acid residue (hydrophilic residue preferred, N or T most preferred);

X³⁴ is an amino acid residue (nonfunctional or aromatic residue preferred, A, F or Y most preferred);

X³⁵ is an amino acid residue (acidic residue preferred, E most preferred);

X³⁶ is an amino acid residue (aromatic residue preferred, Y most preferred);

provided that one or more of X¹⁴ through X³⁶ is a cysteine residue.

A preferred PTH/PTHrP modulating domain sequence formula is III

(SEQ ID NO: 4)

J^{N}J⁷J⁸HNLJ¹²KHLJ¹⁶SJ¹⁸J¹⁹RJ²¹EWLRKKLJ^{C}

wherein:

J^{N} is absent or is selected from J¹J²J³J⁴J⁵J⁶, J²J³J⁴J⁵J⁶, J³J⁴J⁵J⁶;

J¹ is an amino acid residue (nonfunctional, hydrophilic, or aromatic residue preferred; A, S or Y most preferred);

J² is an amino acid residue (nonfunctional residue preferred, V most preferred);

J³ is an amino acid residue (hydrophilic residue preferred, S most preferred);

J⁴ is an amino acid residue (acidic residue preferred, E most preferred);

J⁵ is an amino acid residue (nonfunctional residue preferred, I most preferred);

J⁶ is an amino acid residue (basic residue preferred, Q preferred);

J⁷ is an amino acid residue (nonfunctional or aromatic residue preferred, L or F most preferred);

J⁸ is an amino acid residue (nonfunctional residue preferred, M or Nle most preferred);

J¹² is an amino acid residue (nonfunctional or aromatic residue preferred, G or W most preferred);

J¹⁶ is an amino acid residue (nonfunctional or hydrophilic residue preferred, N, S, or A most preferred);

J¹⁸ is an amino acid residue (nonfunctional residue preferred, M, Nle, L, or V most preferred);

J¹⁹ is an acidic or basic residue (E or R preferred);

J²¹ is an amino acid residue (nonfunctional residue preferred, V, M, or Nle most preferred);

J^{C} is absent or is J²⁹, J²⁹J³⁰, J²⁹J³⁰J³¹, J²⁹J³⁰J³¹J³², J²⁹J³⁰J³¹J³²J³³, or J²⁹J³⁰J³¹J³²J³³J³⁴;

J²⁹ is an amino acid residue (hydrophilic or nonfunctional residue preferred, Q or A most preferred);

J³⁰ is an amino acid residue (hydrophilic or acidic residue preferred, D or E most preferred);

J³¹ is an amino acid residue (lipophilic or nonfunctional residue preferred, V or I most preferred);

J³² is an amino acid residue (basic residue preferred, H most preferred);

J³³ is an amino acid residue (acidic residue preferred, N most preferred);

J³⁴ is an amino acid residue (aromatic residue preferred, F or Y most preferred);

provided that one or more of J¹⁴ through the C-terminal residue is a cysteine residue.

From the formula of SEQ ID NO: 4, peptides appearing in Table 1A or 1B below are most preferred.

Another preferred PTH/PTHrP modulating domain sequence is IV

(SEQ ID NO: 5)

O^{N}LHO¹⁰O¹¹O¹²KSIO¹⁵O¹⁶LRRRFO²³LHHLIO^{C}

wherein:

O^{N} is absent or is YO¹O²O³O⁴O⁵O⁶O⁷, O¹O²O³O⁴O⁵O⁶O⁷, O²O³O⁴O⁵O⁶O⁷, O³O⁴O⁵O⁶O⁷, O⁴O⁵O⁶O⁷, O⁵O⁶O⁷, O⁶O⁷, or O⁷;

O¹ is an amino acid residue (nonfunctional residue preferred, A most preferred);

O² is an amino acid residue (nonfunctional residue preferred, V most preferred);

O³ is an amino acid residue (hydrophilic residue preferred, S most preferred);

O⁴ is an amino acid residue (acidic residue preferred, E most preferred);

O⁵ is an amino acid residue (basic or nonfunctional residue preferred, H or I preferred);

O⁶ is an amino acid residue (hydrophilic residue preferred, Q most preferred);

O⁷ is an amino acid residue (nonfunctional residue preferred, L most preferred);

O¹⁰ is an amino acid residue (acidic or hydrophilic residue preferred, N or D most preferred);

O¹¹ is an amino acid residue (basic or nonfunctional residue preferred, K or L most preferred);

O¹² is an amino acid residue (aromatic or nonfunctional residue preferred, G, F, or W most preferred);

O¹⁵ is an amino acid residue (hydrophilic or nonfunctional residue preferred, I or S most preferred);

O¹⁶ is an amino acid residue (hydrophilic residue preferred, Q or N most preferred);

O¹⁷ is an amino acid residue (acidic or nonfunctional residue preferred, D or L most preferred);

O²³ is an amino acid residue (aromatic residue preferred, with F or W most preferred);

O^{C} is absent or is O²⁹, O²⁹O³⁰, O²⁹O³⁰O³¹, O²⁹O³⁰O³¹O³², O²⁹O³⁰O³¹O³²O³³, O²⁹O³⁰O³¹O³²O³³O³⁴, O²⁹O³⁰O³¹O³²O³³O³⁴O³⁵, or O²⁹O³⁰O³¹O³²O³³O³⁴O³⁵O³⁶; and

O²⁹ through O³⁶ are each independently amino acid residues; provided that one or more of O¹⁴ through the C-terminal residue is a cysteine residue.

From the formula of SEQ ID NO: 5, peptides appearing in Table 2 below are most preferred.

Exemplary peptide sequences for this invention appear in Tables 1A, 1B and 2 below. These peptides may be prepared as described in the cited references or in U.S. Pat. Nos. 4,423,037, 4,968,669, 5,001,22, and 6,051,686, each of which is hereby incorporated by reference in its entirety, or as described hereinafter. Molecules of this invention incorporating these peptide sequences may be prepared by methods known in the art or as described hereinafter. Single letter amino acid abbreviations are used. Any of these peptides may be linked in tandem (i.e., sequentially), with or without linkers. Any peptide containing a cysteinyl residue may be cross-linked with another Cys-containing peptide, either or both of which may be linked to a vehicle. Any peptide having more than one Cys residue may form an intrapeptide disulfide bond, as well. Any of these peptides may be derivatized as described hereinafter.

TABLE 1A

PTH/PTHrP modulating domains based on PTH

| | | SEQ |
|---|---|---|
| | | ID |
| Description | Sequence | NO: |
| human | SVSEIQLMHNLGKHLNSMERVEWLRKKLQDVHNFV | 10 |
| PTH(1-84)¹ | ALGAPLAPRDAGSQRPRKKEDNVLVESHEKSLGEA | |
| | DKADVNVLTKAKSQ | |
| rat | AVSEIQLMHNLGKHLASVERMQWLRKKLQDVHNFV | 11 |
| PTH(1-84)² | SLGVQMAAREGSYQRPTKKEDNVLVDGNSKSLGEG | |
| | DKADVDVLVKAKSQ | |
| human | LMHNLGKHLNSMERVEWLRKKLQDVHNFVALGAPL | 12 |
| PTH³(7-84) | APRDAGSQRPRKKEDNVLVESHEKSLGEADKADVN | |
| | VLTKAKSQ | |
| human | SVSEIQLMHNLGKHLNSMERVEWLRKKLQDVHNFV | 13 |
| PTH(1-44)³ | ALGAPLAPR | |
| human | SVSEIQLMHNLGKHLNSMERVEWLRKKLQDVHNFV | 14 |
| PTH(1-38)³ | ALG | |
| human | VSEIQLMHNLGKHLNSMERVEWLRKKLQDVHNFVA | 15 |
| PTH(2-38)³ | LG | |
| human | SVSEIQLMHNLGKHLNSMERVEWLRKKLQDVHNF | 16 |
| PTH(1-34)⁴ | | |
| [Arg11] | SVSEIQLMHNRGKHLNSMERVEWLRKKLQDVHNF | 17 |
| human | | |
| PTH(1-34) | | |
| [Lys11] | SVSEIQLMHNKGKHLNSMERVEWLRKKLQDVHNF | 18 |
| human | | |
| PTH(1-34) | | |
| [Arg19] | SVSEIQLMHNLGKHLNSMRRVEWLRKKLQDVHNF | 19 |
| human | | |
| PTH(1-34) | | |
| [Tyr1] | YVSEIQLMHNLGKHLNSMERVEWLRKKLQDVHNF | 20 |
| human | | |
| PTH(1-34)³ | | |
| [Leu(8, | SVSEIQLLHNLGKHLNSLERVEWLRKKLQDVHNY | 21 |
| 18), Tyr34] | | |
| human | | |
| PTH(1-34)³ | | |
| bovine | AVSEIQFMHNLGKHLSSMERVEWLRKKLQDVHNF | 22 |
| PTH(1-34)⁵ | | |
| [Leu(8, | AVSEIQFLHNLGKHLSSLERVEWLRKKLQDVHNY | 23 |
| 18), Tyr34] | | |
| bovine | | |
| PTH(1-34)⁶ | | |
| porcine | SVSEIQLMHNLGKHLSSLERVEWLRKKLQDVHNF | 24 |
| PTH(1-34)³ | | |
| rat | AVSEIQLMHNLGKHLASVERMQWLRKKLQDVHNF | 25 |
| PTH(1-34)³ | | |
| [Leu(8, | AVSEIQLLHNLGKHLASVERLQWLRKKLQDVHNY | 26 |
| 21), Tyr34] | | |
| rat | | |
| PTH(1-34)³ | | |
| human | SVSEIQLMHNLGKHLNSMERVEWLRKKLQDV | 27 |
| PTH(1-31)⁷ | | |
| [Leu27] | SVSEIQLMHNLGKHLNSMERVEWLRKLLQDV | 28 |
| human | | |
| PTH(1-31)⁸ | | |
| [Leu(8, | SEIQLLHNLGKHLNSLERVEWLRKKLQDVHNY | 29 |
| 18) Tyr34] | | |
| PTH(3-34)⁹ | | |
| bovine | SEIQFMHNLGKHLSSMERVEWLRKKLQDVHNF | 30 |
| PTH(3-34)¹⁰ | | |
| [Leu(8, | SEIQFLHNLGKHLSSLERVEWLRKKLQDVHNY | 31 |
| 18), Tyr34] | | |
| bovine | | |
| PTH(3-34)¹¹ | | |
| human | LMHNLGKHLNSMERVEWLRKKLQDVHNF | 32 |
| PTH(7-34)¹² | | |
| [Leu(8, | LLHNLGKHLNSLERVEWLRKKLQDVHNY | 33 |
| 18) Tyr34] | | |
| human | | |
| PTH(7-34)⁹ | | |
| bovine | FMHNLGKHLSSMERVEWLRKKLQDVHNF | 34 |
| PTH(7-34)¹³ | | |
| [Tyr34] | FMHNLGKHLSSMERVEWLRKKLQDVHNY | 35 |
| bovine | | |
| PTH(7-34)¹⁴ | | |
| [Leu(8, | FLHNLGKHLSSLERVEWLRKKLQDVHNY | 36 |
| 18), Tyr34] | | |
| bovine | | |
| PTH(7-34)¹⁵ | | |
| [Leu(8, | FLHNLWKHLSSLERVEWLRKKLQDVHNY | 37 |
| 18), Trp12, | | |
| Tyr34] | | |
| bovine | | |
| PTH(7-34)¹⁶ | | |
| [D-Trp12, | FMHNL-D-Trp-KHLSSMERVEWLRKKLQDVHNY | 38 |
| Tyr34] | | |
| bovine | | |
| PTH(7-34)¹⁷ | | |
| human | SVSEIQLMHNLGKHLNSMERVEWLRKKLQD | 39 |
| PTH(1-30) | | |
| [Arg11] | SVSEIQLMHNRGKHLNSMERVEWLRKKLQD | 40 |
| human | | |
| PTH(1-30) | | |
| [Lys11] | SVSEIQLMHNKGKHLNSMERVEWLRKKLQD | 41 |
| human | | |
| PTH(1-30) | | |
| [Arg19] | SVSEIQLMHNLGKHLNSMRRVEWLRKKLQD | 42 |
| human | | |
| PTH(1-30) | | |
| [Tyr1] | YVSEIQLMHNLGKHLNSMERVEWLRKKLQD | 43 |
| human | | |
| PTH(1-30) | | |
| [Leu(8, | SVSEIQLLHNLGKHLNSLERVEWLRKKLQD | 44 |
| 18)] human | | |
| PTH(1-30) | | |
| bovine | AVSEIQFMHNLGKHLSSMERVEWLRKKLQD | 45 |
| PTH(1-30) | | |
| [Leu(8, | AVSEIQFLHNLGKHLSSLERVEWLRKKLQD | 46 |
| 18)] bovine | | |
| PTH(1-30) | | |
| porcine | SVSEIQLMHNLGKHLSSLERVEWLRKKLQD | 47 |
| PTH(1-30) | | |
| rat | AVSEIQLMHNLGKHLASVERMQWLRKKLQD | 48 |
| PTH(1-30) | | |
| [Leu(8, | AVSEIQLLHNLGKHLASVERLQWLRKKLQD | 49 |
| 21), Tyr34] | | |
| rat | | |
| PTH(1-30) | | |
| [Leu27] | SVSEIQLMHNLGKHLNSMERVEWLRKLLQD | 50 |
| human | | |
| PTH(1-30) | | |
| human | SVSEIQLMHNLGKHLNSMERVEWLRKKLQ | 51 |
| PTH(1-29) | | |
| human | SVSEIQLMHNLGKHLNSMERVEWLRKKL | 52 |
| PTH(1-28) | | |
| [Leu(8, | SEIQLLHNLGKHLNSLERVEWLRKKLQD | 53 |
| 18)] | | |
| PTH(3-30) | | |
| bovine | SEIQFMHNLGKHLSSMERVEWLRKKLQD | 54 |
| PTH(3-30) | | |
| [Leu(8, | SEIQFLHNLGKHLSSLERVEWLRKKLQD | 55 |
| 18)] | | |
| bovine | | |
| PTH(3-30) | | |
| human | LMHNLGKHLNSMERVEWLRKKLQD | 56 |
| PTH(7-30) | | |
| [Leu(8, | LLHNLGKHLNSLERVEWLRKKLQD | 57 |
| 18)]human | | |
| PTH(7-30) | | |
| bovine | FMHNLGKHLSSMERVEWLRKKLQD | 58 |
| PTH(7-30) | | |
| [Leu(8, | FLHNLGKHLSSLERVEWLRKKLQD | 59 |
| 18)] | | |
| bovine | | |
| PTH(7-30) | | |
| [Leu(8, | FLHNLWKHLSSLERVEWLRKKLQD | 60 |
| 18), Trp12] | | |
| bovine | | |
| PTH(7-30) | | |
| [D-Trp12] | FMHNL-D-Trp-KHLSSMERVEWLRKKLQD | 61 |
| bovine | | |
| PTH(7-30) | | |

¹Hendy et al. (1981), Proc. Natl. Acad. Sci USA 78: 7365; Kimura et al. (1983), Biochem. Biophys. Res. Commun. 114: 493; Zanelli et al. (1985), Endocrinology 117: 1962; Wingender et al. (1985), J. Biol. Chem. 264: 4367.
²Heinrich et al. (1984), J. Biol. Chem. 259: 3320.
³Bachem Catalogue (1999).
⁴Doppelt et al. (1981), Calcif. Tissue Int. 33: 649; Podbesek et al. (1983) Endocrinology 112: 1000; Kent et al. (1985), Clin. Sci. 68: 171; McKee and Caulfield (1989), Peptide Res. 2: 161; Lee and Russell (1989); Biopolymers 28: 1115; Reeve et al. (1990), Br. Med. J. 301: 314; Neugebauer et al. (1994), Int. J. Peptide Protein Res. 43: 555.
⁵Nakamura et al. (1981); Proc. Soc. Exp. Biol. Med. 168: 168; Law et al. (1983), J. Clin. Endocrinol. Metab. 56: 1335; Wang et al. (1984), Eur. J. Pharmacol. 97, 209; Sham et al. (1986), Gen. Comp. Endocrinol. 61: 148; Smith et al. (1987), Arch. Biochem. Biophys. 253: 81.
⁶Based on Coltrera et al. (1981), J. Biol. Chem. 256: 10555; Bergeron et al. (1981), Endocrinology 109: 1552.
⁷Jouishomme et al. (1994), J. Bone Miner. Res. 9: 943; Whitfield and Morley; TIPS 16: 382.
⁸Barbier et al. (1997), J. Med. Chem. 40: 1373.
⁹Based on Schipani et al. (1993), Endocrinology 132: 2157-65.
¹⁰Scharla et al. (1991), Horm. Metab. Res. 23: 66-9; McGowan et al. (1983), Science 219: 67; Lowik et al. (1985), Cell Calcium 6: 311.
¹¹Based on Jobert et al. (1997), Endocrinology 138: 5282; Schipani et al. (1993); Rosenblatt et al. (1977), J. Biol. Chem. 252: 5847; Segre et al. (1979), J. Biol. Chem. 254: 6980; Nussbaum et al. (1980), J. Biol. Chem. 225: 10183; Gray et al. (1980), Br. J. Pharmac. 76: 259.
¹²Nissenson et al. (1999), Endocrinology 140: 1294-1300.
¹³Jueppner et al. (1996), Endocrinology.
¹⁴Horiuchi et al. (1983), Science 220: 1053.
¹⁵Schipani et al. (1993); Holick et al. (1995), Bone 16: 140S (abstract 223, Conference, Melbourne, February 1995).
¹⁶Based on Dresner-Pollak et al. (1996), JBMR 11: 1061-5.
¹⁷Goldman et al. (1988), Endocrinology 123: 2597.

TABLE 1B

PTH/PTHrP modulating domains based on Cys
modifications of PTH

| | | SEQ |
|---|---|---|
| | | ID |
| Description | Sequence | NO: |
| Cys33 PTH(1-34) | SVSEI QLMHN LGKHL NSMER VEWLR | 172 |
| (Cys-33 inser- | KKLQD VHCNF | |
| tion) | | |
| Cys27, 33 PTH(1- | SVSEI QLMHN LGKHL NSMER VEWLR | 173 |
| 34)(Cys-27 re- | KCLQD VHCNF | |
| placement, Cys- | | |
| 33 insertion) | | |
| Cys-33 replace- | SVSEI QLMHN LGKHL NSMER VEWLR | 174 |
| ment | KKLQD VHCF | |
| CGPTH 4 Cys-34 | SVSEI QLMHN LGKHL NSMER VEWLR | 175 |
| replacement | KKLQD VHNC | |
| Cys14 PTH(1-34) | SVSEI QLMHN LGKCL NSMER VEWLR | 176 |
| | KKLQD VHNF | |
| Cys15 PTH(1-34) | SVSEI QLMHN LGKHC NSMER VEWLR | 177 |
| | KKLQD VHNF | |
| Cys16 PTH(1-34) | SVSEI QLMHN LGKHL CSMER VEWLR | 178 |
| | KKLQD VHNF | |
| Cys17 PTH(1-34) | SVSEI QLMHN LGKHL NCMER VEWLR | 179 |
| | KKLQD VHNF | |
| Cys18 PTH(1-34) | SVSEI QLMHN LGKHL NSCER VEWLR | 180 |
| | KKLQD VHNF | |
| Cys19 PTH(1-34) | SVSEI QLMHN LGKHL NSMCR VEWLR | 181 |
| | KKLQD VHNF | |
| Cys20 PTH(1-34) | SVSEI QLMHN LGKHL NSMEC VEWLR | 182 |
| | KKLQD VHNF | |
| Cys21 PTH(1-34) | SVSEI QLMHN LGKHL NSMER CEWLR | 183 |
| | KKLQD VHNF | |
| Cys22 PTH(1-34) | SVSEI QLMHN LGKHL NSMER VCWLR | 184 |
| | KKLQD VHNF | |
| Cys24 PTH(1-34) | SVSEI QLMHN LGKHL NSMER VEWCR | 185 |
| | KKLQD VHNF | |
| Cys25 PTH(1-34) | SVSEI QLMHN LGKHL NSMER VEWLC | 186 |
| | KKLQD VHNF | |
| Cys26 PTH(1-34) | SVSEI QLMHN LGKHL NSMER VEWLR | 187 |
| | CKLQD VHNF | |
| Cys27 PTH(1-34) | SVSEI QLMHN LGKHL NSMER VEWLR | 188 |
| | KCLQD VHNF | |
| Cys28 PTH(1-34) | SVSEI QLMHN LGKHL NSMER VEWLR | 189 |
| | KKCQD VHNF | |
| Cys29 PTH(1-34) | SVSEI QLMHN LGKHL NSMER VEWLR | 190 |
| | KKLCD VHNF | |
| Cys30 PTH(1-34) | SVSEI QLMHN LGKHL NSMER VEWLR | 191 |
| | KKLQC VHNF | |
| Cys31 PTH(1-34) | SVSEI QLMHN LGKHL NSMER VEWLR | 192 |
| | KKLQD CHNF | |
| Cys32 PTH(1-34) | SVSEI QLMHN LGKHL NSMER VEWLR | 193 |
| | KKLQD VCNF | |

TABLE 2

PTH/PTHrP modulating domains based on PTHrP

| | | |
|---|---|---|
| | | SEQ |
| Description | Sequence | ID NO: |

| | | |
|---|---|---|
| human PTHrP(1-86)³ | AVSEHQLLHDKGKSIQDLRRRFFLHHLIAEIHTAE | 62 |
| | IRATSEVSPNSKPSPNTKNHPVRFGSDDEGRYLTQ | |
| | ETNKVETYKEQPLKTP | |
| human PTHrP(1-34)¹⁸ | AVSEHQLLHDKGKSIQDLRRRFFLHHLIAEIHTA | 63 |
| [Tyr36] human PTHrP(1-36)³ | AVSEHQLLHDKGKSIQDLRRRFFLHHLIAEIHTAE | 64 |
| | Y | |
| [Ile5, Trp23, Tyr36] human | AVSEIQLLHDKGKSIQDLRRRFWLHHLIAEIHTAE | 65 |
| PTHrP(1-36)³ | Y | |
| Tyr-human PTHrP(1-34)³ | YAVSEHQLLHDKGKSIQDLRRRFFLHHLIAEIHTA | 66 |
| [Asn10, Leu11, D-Phe12] | AVSEHQLLHNL-D-Phe- | 67 |
| human PTHrP(1-34)¹⁹ | KSIQDLRRRFFLHHLIAEIHTA | |
| PTHrP(7-34)²⁰ | LLHDKGKSIQDLRRRFFLHHLIAEIHTA | 68 |
| [Asn10, Leu11] human | LLHNLGKSIQDLRRRFFLHHLIAEIHTA | 69 |
| PTHrP(7-34) | | |
| [Asn16, Leu17] PTHrP(7-34)²¹ | LLHDKGKSINLLRRRFFLHHLIAEIHTA | 70 |
| [Leu11, D-Trp12] human | LLHDL-D-Trp-KSIQDLRRRFFLHHLIAEIHTA | 71 |
| PTHrP(7-34)²² | | |
| [Asn10, Leu11, D-Trp12] | LLHNL-D-Trp-KSIQDLRRRFFLHHLIAEIHTA | 72 |
| PTHrP(7-34)²³ | | |
| [D-Trp12] PTHrP(8-34) | LHNL-D-Trp-KSIQDLRRRFFLHHLIAEIHTA | 73 |
| [D-Phe12] PTHrP(8-34) | LHNL-D-Phe-KSIQDLRRRFFLHHLIAEIHTA | 74 |
| [Asn10, Leu11, D-Trp12] | LLHNL-D-Trp-KSIQDLRRRFFLHHLIAEIHTA | 75 |
| human PTHrP(7-34)²⁰ | | |
| human PTHrP(1-30) | AVSEHQLLHDKGKSIQDLRRRFFLHHLIAE | 76 |
| [Ile5, Trp23] human | AVSEIQLLHDKGKSIQDLRRRFWLHHLIAE | 77 |
| PTHrP(1-30) | | |
| Tyr-human PTHrP(1-30) | YAVSEHQLLHDKGKSIQDLRRRFFLHHLIAE | 78 |
| [Asn10, Leu11, D-Phe12] | AVSEHQLLHNL-D-Phe- | 79 |
| human PTHrP(1-30) | KSIQDLRRRFFLHHLIAE | |
| PTHrP(7-30) | LLHDKGKSIQDLRRRFFLHHLIAE | 80 |
| [Asn10, Leu11] human | LLHNLGKSIQDLRRRFFLHHLIAE | 81 |
| PTHrP(7-30) | | |
| [Asn16, Leu17] PTHrP(7-30) | LLHDKGKSINLLRRRFFLHHLIAE | 82 |
| [Leu11, D-Trp12] human | LLHDL-D-Trp-KSIQDLRRRFFLHHLIAE | 83 |
| PTHrP(7-30) | | |
| [Asn10, Leu11, D-Trp12] | LLHNL-D-Trp-KSIQDLRRRFFLHHLIAE | 84 |
| PTHrP(7-30) | | |
| [D-Trp12] PTHrP(8-30) | LHNL-D-Trp-KSIQDLRRRFFLHHLIAE | 85 |
| [D-Phe12] PTHrP(8-30) | LHNL-D-Phe-KSIQDLRRRFFLHHLIAE | 86 |
| [Asn10, Leu11, D-Trp12] | LLHNL-D-Trp-KSIQDLRRRFFLHHLIAE | 87 |
| human PTHrP(7-30) | | |
| [Haa(Laa Laa Haa Haa)2 Laa | SVSEIQLMHNLGKHLNSMERVELLEKLLEKLHNF | 88 |
| 22-31] human PTH(1-34)²⁴ | | |
| [Haa(Laa Laa Haa Haa)2 Laa | SVSEIQLMHNLGKHLNSMERVELLEKLLKKLHNF | 89 |
| 22-31] human PTH(1-34)²⁴ | | |
| [Haa(Laa Laa Haa Haa)2 Laa | SVSEIQLMHNLGKHLNSMERVALAEALAEALHNF | 90 |
| 22-31] human PTH(1-34)²⁵ | | |
| [Haa(Laa Laa Haa Haa)2 Laa | SVSEIQLMHNLGKHLNSMERVSLLSSLLSSLHNF | 91 |
| 22-31] human PTH(1-34)²⁶ | | |
| [Haa(Laa Laa Haa Haa)2 Laa | SVSEIQLMHNLGKHLNSMERVAFYDKVAEKLHNF | 92 |
| 22-31] human PTH(1-34)²⁷ | | |
| [Haa(Laa Laa Haa Haa)₂ Laa | LMHNLGKHLNSMERVELLEKLLEKLHNF | 93 |
| 22-31] human PTH(7-34)²⁴ | | |
| [Haa(Laa Laa Haa Haa)₂ Laa | LMHNLGKHLNSMERVELLEKLLKKLHNF | 94 |
| 22-31] human PTH(7-34)²⁴ | | |
| [Haa(Laa Laa Haa Haa)2 Laa | LMHNLGKHLNSMERVALAEALAEALHNF | 95 |
| 22-31] human PTH(7-34)²⁵ | | |
| [Haa(Laa Laa Haa Haa)2 Laa | LMHNLGKHLNSMERVSLLSSLLSSLHNF | 96 |
| 22-31] human PTH(7-34)²⁶ | | |
| [Haa(Laa Laa Haa Haa)2 Laa | LMHNLGKHLNSMERVAFYDKVAEKLHNF | 97 |
| 22-31] human PTH(7-34)²⁷ | | |
| [Haa(Laa Laa Haa Haa)2 Laa | AVSEHQLLHDKGKSIQDLRRRELLEKLLEKLHTA | 98 |
| 22-31] human PTHrP(1-34)²⁴ | | |
| [Haa(Laa Laa Haa Haa)2 Laa | AVSEHQLLHDKGKSIQDLRRRELLEKLLKKLHTA | 99 |
| 22-31] human PTHrP(1-34)²⁴ | | |
| [Haa(Laa Laa Haa Haa)2 Laa | AVSEHQLLHDKGKSIQDLRRRALAEALAEALHTA | 100 |
| 22-31] human PTHrP(1-34)²⁵ | | |
| [Haa(Laa Laa Haa Haa)2 Laa | AVSEHQLLHDKGKSIQDLRRRSLLSSLLSSLHTA | 101 |
| 22-31] human PTHrP(1-34)²⁶ | | |
| [Haa(Laa Laa Haa Haa)2 Laa | AVSEHQLLHDKGKSIQDLRRRAFYDKVAEKLHTA | 102 |
| 22-31] human PTHrP(1-34)²⁷ | | |
| [Haa(Laa Laa Haa Laa Haa | LLHDKGKSIQDLRRRELLEKLLEKLHTA | 103 |
| 22-31] human PTHrP(7-34)²⁸ | | |
| [Haa(Laa Laa Haa Haa)2 Laa | LLHDKGKSIQDLRRRELLEKLLKKLHTA | 104 |
| 22-31] human PTHrP(7-34)²⁴ | | |
| [Haa(Laa Laa Haa Haa)2 Laa | LLHDKGKSIQDLRRRALAEALAEALHTA | 105 |
| 22-31] human PTHrP(7-34)²⁵ | | |
| [Haa(Laa Laa Haa Haa)2 Laa | LLHDKGKSIQDLRRRSLLSSLLSSLHTA | 106 |
| 22-31] human PTHrP(7-34)²⁶ | | |
| [Haa(Laa Laa Haa Haa)2 Laa | LLHDKGKSIQDLRRRAFYDKVAEKLHTA | 107 |
| 22-31] human PTHrP(7-34)²⁷ | | |
| [Lys11, Lys13; Arg19, Arg21; | AVSEHQLLHDKGKSIQDLRRRELLEKLLRKLHTA | 108 |
| Haa(Laa Laa Haa Haa)2 Laa | | |
| 22-31] human PTHrP(1-34)²⁹ | | |
| [Lys11, Lys13; Arg19, Arg21; | AVSEHQLLHDKGKSIQDLRRRELLEKLLEKLHTS | 109 |
| Haa(Laa Laa Haa Haa)2 Laa | | |
| 22-31] human PTHrP(1-34)³⁰ | | |
| [Lys11, Lys13; Arg19, Arg21; | AVSEHQLLHDKGKSIQDLRRRELLEKLLEKLHTAG | 110 |
| Haa(Laa Laa Haa Haa)2 Laa | | |
| 22-31] human PTHrP(1-34)³¹ | | |
| [Lys11, Lys13; Arg19, Arg21; | AVSEHQLLHDKGKSIQDLRRRELLEKLLEKLKEL | 111 |
| Haa(Laa Laa Haa Haa)2 Laa | | |
| 22-31] human PTHrP(1-34)³² | | |
| [Lys11, Lys13, Ala19, Arg21, | AVSEHQLLHDKGKSIQDLARRELLEKLLEKLHTA | 112 |
| Haa(Laa Laa Haa Haa)₂ Laa | | |
| 22-31] human PTHrP(1-34)³³ | | |
| [Lys11, Lys13, Arg19, Ala21, | AVSEHQLLHDKGKSIQDLRRAELLEKLLEKLHTA | 113 |
| Haa(Laa Laa Haa Haa)₂ Laa | | |
| 22-31] human PTHrP(1-34)³⁴ | | |
| [Leu11, Lys13, Arg19, Arg21, | AVSEAQLLHDLGKSIQDLRRRELLEKLLEKLHAL | 114 |
| Haa(Laa Laa Haa Haa)₂ Laa | | |
| 22-31] human PTHrP(1-34)³⁵ | | |
| [Lys11, Lys13, Arg19, Arg21, | AVSEHQLLHDKGKSIQDLRRRELLERLLERLHTA | 115 |
| Haa(Laa Laa Haa Haa)₂ Laa | | |
| 22-31] human PTHrP(1-34³⁶ | | |
| [Arg11, Arg13, Arg19, Arg21, | AVSEHQLLHDRGRSIQDRRRELLERLLERLHTA | 116 |
| Haa(Laa Laa Haa Haa)₂ Laa | | |
| 22-31] human PTHrP(1-34)³⁷ | | |
| [Arg11, Lys13, Arg19, Arg21, | AVSEHQLLHDRGKSIQDLRRRELLERLLKRLHTA | 117 |
| Haa(Laa Laa Haa Haa)₂ Laa | | |
| 22-31] human PTHrP(1-34)³⁸ | | |
| [Arg11, Arg13, Arg19, Arg21, | AVSEHQLLHDRGRSIQDLRRRELLERLLKRLHTA | 118 |
| Haa(Laa Laa Haa Haa)₂ Laa | | |
| 22-31] human PTHrP(1-34)³⁹ | | |
| Haa(Laa Laa Haa Haa)₂ Laa | AVSEHQLLHDKGKSIQDLRRRALAEALAEALHTA | 119 |
| 22-31] human PTHrP(1-34)⁴⁰ | | |
| Haa(Laa Laa Haa Haa)₂ Laa | AVSEHQLLHDKGKSIQDLRRRSLLSSLLSSLHTA | 120 |
| 22-31] human PTHrP(1-34)⁴¹ | | |
| Haa(Laa Laa Haa Haa)₂ Laa | AVSEHQLLHDKGKSIQDLRRRAFYDKVAEKLHTA | 121 |
| 22-31] human PTHrP(1-34)⁴² | | |
| Haa(Laa Laa Haa Haa)₂ Laa | AVSEIQFMHNLGKHLSSMERVELLEKLLEKLHNY | 122 |
| 22-31] human PTHrP(1-34)⁴³ | | |
| Haa(Laa Laa Haa Haa)₂ Laa | AVSEIQFMHNLGKHLSSMRRRELLEKLLEKLHNY | 123 |
| 22-31] human PTHrP(1-34)⁴⁴ | | |
| [Haa(Laa Laa Haa Haa)₂ Laa | SVSEIQLMHNLGKHLNSMERVELLEKLLEK | 124 |
| 22-30] human PTH(1-30) | | |
| [Haa(Laa Laa Haa Haa)₂ Laa | SVSEIQLMHNLGKHLNSMERVELLEKLLKK | 125 |
| 22-30] human PTH(1-30) | | |
| [Haa(Laa Laa Haa Haa)₂ Laa | SVSEIQLMHNLGKHLNSMERVALAEALAEA | 126 |
| 22-30] human PTH(1-30) | | |
| [Haa(Laa Laa Haa Haa)₂ Laa | SVSEIQLMHNLGKHLNSMERVSLLSSLLSS | 127 |
| 22-30] human PTH(1-30) | | |
| [Haa(Laa Laa Haa Haa)₂ Laa | SVSEIQLMHNLGKHLNSMERVAFYDKVAEKLHNF | 128 |
| 22-30] human PTH(1-34)²⁷ | | |
| [Haa(Laa Laa Haa Haa)₂ Laa | LMHNLGKHLNSMERVELLEKLLEK | 129 |
| 22-30] human PTH(7-30) | | |
| [Haa(Laa Laa Haa Haa)₂ Laa | LMHNLGKHLNSMERVELLEKLLKK | 130 |
| 22-30] human PTH(7-30) | | |
| [Haa(Laa Laa Haa Haa)₂ Laa | LMHNLGKHLNSMERVALAEALAEA | 131 |
| 22-30] human PTH(7-30) | | |
| [Haa(Laa Laa Haa Haa)₂ Laa | LMHNLGKHLNSMERVSLLSSLLSS | 132 |
| 22-30] human PTH(7-30) | | |
| [Haa(Laa Laa Haa Haa)₂ Laa | LMHNLGKHLNSMERVAFYDKVAEK | 133 |
| 22-30] human PTH(7-30) | | |
| [Haa(Laa Laa Haa Haa)₂ Laa | AVSEHQLLHDKGKSIQDLRRRELLEKLLEK | 134 |
| 22-30] human PTHrP(1-30) | | |
| [Haa(Laa Laa Haa Haa)₂ Laa | AVSEHQLLHDKGKSIQDLRRRELLEKLLKK | 135 |
| 22-30] human PTHrP(1-30) | | |
| [Haa(Laa Laa Haa Haa)₂ Laa | AVSEHQLLHDKGKSIQDLRRRALAEALAEA | 136 |
| 22-30] human PTHrP(1-30) | | |
| [Haa(Laa Laa Haa Haa)₂ Laa | AVSEHQLLHDKGKSIQDLRRRSLLSSLLSS | 137 |
| 22-30] human PTHrP(1-30) | | |
| [Haa(Laa Laa Haa Haa)₂ Laa | AVSEHQLLHDKGKSIQDLRRRAFYDKVAEK | 138 |
| 22-30] human PTHrP(1-30) | | |
| [Haa(Laa Laa Haa Haa)₂ Laa | LLHDKGKSIQDLRRRELLEKLLEK | 139 |
| 22-30] human PTHrP(7-30) | | |
| [Haa(Laa Laa Haa Haa)₂ Laa | LLHDKGKSIQDLRRRELLEKLLKK | 140 |
| 22-30] human PTHrP(7-30) | | |
| [Haa(Laa Laa Haa Haa)₂ Laa | LLHDKGKSIQDLRRRALAEALAEA | 141 |
| 22-30] human PTHrP(7-30) | | |
| [Haa(Laa Laa Haa Haa)₂ Laa | LLHDKGKSIQDLRRRSLLSSLLSS | 142 |
| 22-30] human PTHrP(7-30) | | |
| [Haa(Laa Laa Haa Haa)₂ Laa | LLHDKGKSIQDLRRRAFYDKVAEK | 143 |
| 22-30] human PTHrP(7-30) | | |
| [Lys11, Lys13; Arg19, Arg21; | AVSEHQLLHDKGKSIQDLRRRELLEKLLRK | 144 |
| Haa(Laa Laa Haa Haa)₂ Laa | | |
| 22-30] human PTHrP(1-30) | | |
| [Lys11, Lys13; Arg19, Arg21; | AVSEHQLLHDKGKSIQDLRRRELLEKLLEK | 145 |
| Haa(Laa Laa Haa Haa)₂ Laa | | |
| 22-30] human PTHrP(1-30) | | |
| [Lys11, Lys13; Arg19, Arg21; | AVSEHQLLHDKGKSIQDLRRRELLEKLLEKLHT | 146 |
| Haa(Laa Laa Haa Haa)₂ Laa | | |
| 22-30] human PTHrP(1-30) | | |
| [Lys11, Lys13; Arg19, Arg21; | AVSEHQLLHDKGKSIQDLRRRELLEKLLEK | 147 |
| Haa(Laa Laa Haa Haa)₂ Laa | | |
| 22-30] human PTHrP(1-30) | | |
| [Lys11, Lys13, Ala19, Arg21, | AVSEHQLLHDKGKSIQDLARRELLEKLLEK | 148 |
| Haa(Laa Laa Haa Haa)₂ Laa | | |
| 22-30] human PTHrP(1-30) | | |
| [Lys11, Lys13, Arg19, Ala21, | AVSEHQLLHDKGKSIQDLRRAELLEKLLEK | 149 |
| Haa(Laa Laa Haa Haa)₂ Laa | | |
| 22-30] human PTHrP(1-30) | | |
| [Leu11, Lys13, Arg19, Arg21, | AVSEAQLLHDLGKSIQDLRRRELLEKLLEK | 150 |
| Haa(Laa Laa Haa Haa)₂ Laa | | |
| 22-30] human PTHrP(1-30) | | |
| [Lys11, Lys13, Arg19, Arg21, | AVSEHQLLHDKGKSIQDLRRRELLERLLER | 151 |
| Haa(Laa Laa Haa Haa)₂ Laa | | |
| 22-30] human PTHrP(1-30) | | |
| [Arg11, Arg13, Arg19, Arg21, | AVSEHQLLHDRGRSIQDRRRELLERLLER | 152 |
| Haa(Laa Laa Haa Haa)₂ Laa | | |
| 22-30] human PTHrP(1-30) | | |
| [Arg11, Lys13, Arg19, Arg21, | AVSEHQLLHDRGKSIQDLRRRELLERLLKR | 153 |
| Haa(Laa Laa Haa Haa)₂ Laa | | |
| 22-30] human PTHrP(1-30) | | |
| [Arg11, Arg13, Arg19, Arg21, | AVSEHQLLHDRGRSIQDLRRRELLERLLKR | 154 |
| Haa(Laa Laa Haa Haa)₂ Laa | | |
| 22-30] human PTHrP(1-30) | | |
| Haa(Laa Laa Haa Haa)₂ Laa | AVSEHQLLHDKGKSIQDLRRRALAEALAEA | 155 |
| 22-30] human PTHrP(1-30) | | |
| Haa(Laa Laa Haa Haa)₂ Laa | AVSEHQLLHDKGKSIQDLRRRSLLSSLLSS | 156 |
| 22-30] human PTHrP(1-30) | | |
| Haa(Laa Laa Haa Haa)₂ Laa | AVSEHQLLHDKGKSIQDLRRRAFYDKVAEK | 157 |
| 22-30] human PTHrP(1-30) | | |
| Haa(Laa Laa Haa Haa)₂ Laa | AVSEIQFMHNLGKHLSSMERVELLEKLLEK | 158 |
| 22-30] human PTHrP(1-30) | | |
| Haa(Laa Laa Haa Haa)₂ Laa | AVSEIQFMHNLGKHLSSMRRRELLEKLLEK | 159 |
| 22-30] human PTHrP(1-30) | | |

¹⁸Moseley et al. (1987), Proc. Natl. Acad. Sci. USA 84: 5048; Suva et al. (1987), Science 237: 893; Kemp et al. (1987), Science 238: 1568; Paspaliaris et al. (1995), Bone 16: 141S (abstract 225, Conference, Melbourne 1995).
¹⁹Based on JP 07316195, May 25, 1994 (Nippon Kayaku).
²⁰Nagasaki et al. (1989), Biochem. Biophys. Res. Commun. 158: 1036; Nutt et al.; Endocrinology 127, 491 (1990).
²¹Williams et al. (1998), J. Reproduction & Fertility 112: 59-67.
²²Gardella et al. (1996), Endocrinol. 137: 3936-41; Fukayama et al. (1998), Am. J. Physiol. 274: E297-E303.
²³Li et al. (1996), Endocrinology.
²⁴Incorporating SEQ ID NO: 26 from U.S. Pat. No. 6,051,686.
²⁵Incorporating SEQ ID NO: 28 from U.S. Pat. No. 6,051,686.
²⁶Incorporating SEQ ID NO: 29 from U.S. Pat. No. 6,051,686.
²⁷Incorporating SEQ ID NO: 30 from U.S. Pat. No. 6,051,686.
²⁸Incorporating SEQ ID NO: 26 from U.S. Pat. No. 6,051,686
²⁹Incorporating SEQ ID NO: 5 from U.S. Pat. No. 6,051,686.
³⁰Based on SEQ ID NOS: 8, 9 from U.S. Pat. No. 6,051,686
³¹Incorporating SEQ ID NO: 10 from U.S. Pat. No. 6,051,686
³²Incorporating SEQ ID NO: 11 from U.S. Pat. No. 6,051,686
³³Incorporating SEQ ID NO: 12 from U.S. Pat. No. 6,051,686
³⁴Incorporating SEQ ID NO: 12 from U.S. Pat. No. 6,051,686
³⁵Incorporating SEQ ID NO: 14 from U.S. Pat. No. 6,051,686
³⁶Incorporating SEQ ID NO: 15 from U.S. Pat. No. 6,051,686
³⁷Incorporating SEQ ID NO: 16 from U.S. Pat. No. 6,051,686
³⁸Incorporating SEQ ID NO: 17 and 18 from U.S. Pat. No. 6,051,686
³⁹Incorporating SEQ ID NO: 19 from U.S. Pat. No. 6,051,686
⁴⁰Incorporating SEQ ID NO: 20 from U.S. Pat. No. 6,051,686
⁴¹Incorporating SEQ ID NO: 21 from U.S. Pat. No. 6,051,686
⁴²Incorporating SEQ ID NO: 22 from U.S. Pat. No. 6,051,686
⁴³Modified from SEQ ID NO: 23 from U.S. Pat. No. 6,051,686
⁴⁴Modified from SEQ ID NO: 24 from U.S. Pat. No. 6,051,686

Another useful PTH/PTHrP modulating domain has the sequence of the peptide known as TIIP39:

(SEQ ID NO: 160)

SLALADDAAFRERARLLAALERRHWLNSYMHKLLVLDAP

TIP39 is described by Usdin et al. (1999), Nature Neurosci. 2(11): 941-3; Usdin et al. (1996), Endocrinology 137(10):4285-97; Usdin et al. (1995), J. Biol. Chem. 270(26): 15455-8; Usdin et al. (1999), Endocrinol. 140(7): 3363-71.

Additional useful PTH/PTHrP modulating domain sequences may result from conservative and/or non-conservative modifications of the amino acid sequences of SEQ ID NOS: 3, 4, 5, TIP39, or the sequences listed in Tables 1A, 1B, and 2. In particular, useful PTH/PTHrP modulating domains further comprise molecules in which any residue at position 14 through the C-terminal amino acid of any sequence in Tables 1A and 2 is substituted with a cysteine residue to provide an attachment site for a polymer (PEG preferred). Cysteine substitutions at position 27 through the C-terminal peptide are preferred.

Conservative modifications will produce peptides having functional and chemical characteristics similar to those of the PTH or PTHrP peptide from which such modifications are made. In contrast, substantial modifications in the functional and/or chemical characteristics of the peptides may be accomplished by selecting substitutions in the amino acid sequence that differ significantly in their effect on maintaining (a) the structure of the molecular backbone in the area of the substitution, for example, as a sheet or helical conformation, (b) the charge or hydrophobicity of the molecule at the target site, or (c) the size of the molecule.

For example, a “conservative amino acid substitution” may involve a substitution of a native amino acid residue with a nonnative residue such that there is little or no effect on the polarity or charge of the amino acid residue at that position. Furthermore, any native residue in the polypeptide may also be substituted with alanine, as has been previously described for “alanine scanning mutagenesis” (see, for example, MacLennan et al., 1998, Acta Physiol. Scand. Suppl. 643:55-67; Sasaki et al., 1998, Adv. Biophys. 35:1-24, which discuss alanine scanning mutagenesis).

Desired amino acid substitutions (whether conservative or non-conservative) can be determined by those skilled in the art at the time such substitutions are desired. For example, amino acid substitutions can be used to identify important residues of the peptide sequence, or to increase or decrease the affinity of the peptide or vehicle-peptide molecules (see preceding formulae) described herein. Exemplary amino acid substitutions are set forth in Table 3.

TABLE 3
Amino Acid Substitutions

| Original | Exemplary | Preferred |
|---|---|---|
| Residues | Substitutions | Substitutions |
| Ala | Val, Leu, Ile | Val |
| Arg | Lys, Gln, Asn | Lys |
| Asn | Gln | Gln |
| Asp | Glu | Glu |
| Cys | Ser, Ala | Ser |
| Gln | Asn | Asn |
| Glu | Asp | Asp |
| Gly | Pro, Ala | Ala |
| His | Asn, Gln, Lys, Arg | Arg |
| Ile | Leu, Val, Met, Ala, | Leu |
| | Phe, Norleucine | |
| Leu | Norleucine, Ile, Val, | Ile |
| | Met, Ala, Phe | |
| Lys | Arg, 1,4 Diamino- | Arg |
| | butyric Acid, Gln, Asn | |
| Met | Leu, Phe, Ile | Leu |
| Phe | Leu, Val, Ile, Ala, Tyr | Leu |
| Pro | Ala | Gly |
| Ser | Thr, Ala, Cys | Thr |
| Thr | Ser | Ser |
| Trp | Tyr, Phe | Tyr |
| Tyr | Trp, Phe, Thr, Ser | Phe |
| Val | Ile, Met, Leu, Phe, | Leu |
| | Ala, Norleucine | |

In certain embodiments, conservative amino acid substitutions also encompass non-naturally occurring amino acid residues which are typically incorporated by chemical peptide synthesis rather than by synthesis in biological systems.

As noted in the foregoing section “Definition of Terms,” naturally occurring residues may be divided into classes based on common sidechain properties that may be useful for modifications of sequence. For example, non-conservative substitutions may involve the exchange of a member of one of these classes for a member from another class. Such substituted residues may be introduced into regions of the peptide that are homologous with non-human orthologs, or into the non-homologous regions of the molecule. In addition, one may also make modifications using P or G for the purpose of influencing chain orientation.

In making such modifications, the hydropathic index of amino acids may be considered. Each amino acid has been assigned a hydropathic index on the basis of their hydrophobicity and charge characteristics, these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is understood in the art. Kyte et al., J. Mol. Biol., 157: 105-131 (1982). It is known that certain amino acids may be substituted for other amino acids having a similar hydropathic index or score and still retain a similar biological activity. In making changes based upon the hydropathic index, the substitution of amino acids whose hydropathic indices are within +2 is preferred, those which are within +1 are particularly preferred, and those within ±0.5 are even more particularly preferred.

It is also understood in the art that the substitution of like amino acids can be made effectively on the basis of hydrophilicity. The greatest local average hydrophilicity of a protein, as governed by the hydrophilicity of its adjacent amino acids, correlates with its immunogenicity and antigenicity, i.e., with a biological property of the protein.

The following hydrophilicity values have been assigned to amino acid residues: arginine (+3.0); lysine (+3.0); aspartate (+3.0±1); glutamate (+3.0+1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). In making changes based upon similar hydrophilicity values, the substitution of amino acids whose hydrophilicity values are within +2 is preferred, those which are within +1 are particularly preferred, and those within ±0.5 are even more particularly preferred. One may also identify epitopes from primary amino acid sequences on the basis of hydrophilicity. These regions are also referred to as “epitopic core regions.”

A skilled artisan will be able to determine suitable variants of the polypeptide as set forth in the foregoing sequences using well known techniques. For identifying suitable areas of the molecule that may be changed without destroying activity, one skilled in the art may target areas not believed to be important for activity. For example, when similar polypeptides with similar activities from the same species or from other species are known, one skilled in the art may compare the amino acid sequence of a peptide to similar peptides. With such a comparison, one can identify residues and portions of the molecules that are conserved among similar polypeptides. It will be appreciated that changes in areas of a peptide that are not conserved relative to such similar peptides would be less likely to adversely affect the biological activity and/or structure of the peptide. One skilled in the art would also know that, even in relatively conserved regions, one may substitute chemically similar amino acids for the naturally occurring residues while retaining activity (conservative amino acid residue substitutions). Therefore, even areas that may be important for biological activity or for structure may be subject to conservative amino acid substitutions without destroying the biological activity or without adversely affecting the peptide structure.

Additionally, one skilled in the art can review structure-function studies identifying residues in similar peptides that are important for activity or structure. In view of such a comparison, one can predict the importance of amino acid residues in a peptide that correspond to amino acid residues that are important for activity or structure in similar peptides. One skilled in the art may opt for chemically similar amino acid substitutions for such predicted important amino acid residues of the peptides.

One skilled in the art can also analyze the three-dimensional structure and amino acid sequence in relation to that structure in similar polypeptides. In view of that information, one skilled in the art may predict the alignment of amino acid residues of a peptide with respect to its three dimensional structure. One skilled in the art may choose not to make radical changes to amino acid residues predicted to be on the surface of the protein, since such residues may be involved in important interactions with other molecules. Moreover, one skilled in the art may generate test variants containing a single amino acid substitution at each desired amino acid residue. The variants can then be screened using activity assays know to those skilled in the art. Such data could be used to gather information about suitable variants. For example, if one discovered that a change to a particular amino acid residue resulted in destroyed, undesirably reduced, or unsuitable activity, variants with such a change would be avoided. In other words, based on information gathered from such routine experiments, one skilled in the art can readily determine the amino acids where further substitutions should be avoided either alone or in combination with other mutations.

A number of scientific publications have been devoted to the prediction of secondary structure. See Moult J., Curr. Op. in Biotech., 7(4): 422-427 (1996), Chou et al., Biochemistry, 13(2): 222-245 (1974); Chou et al., Biochemistry, 113(2): 211-222 (1974); Chou et al., Adv. Enzymol. Relat. Areas Mol. Biol., 47: 45-148 (1978); Chou et al, Ann. Rev. Biochem., 47: 251-276 and Chou et al., Biophys. J., 26: 367-384 (1979). Moreover, computer programs are currently available to assist with predicting secondary structure. One method of predicting secondary structure is based upon homology modeling. For example, two polypeptides or proteins which have a sequence identity of greater than 30%, or similarity greater than 40% often have similar structural topologies. The recent growth of the protein structural data base (PDB) has provided enhanced predictability of secondary structure, including the potential number of folds within a polypeptide's or protein's structure. See Holm et al., Nucl. Acid. Res., 27(1): 244-247 (1999). It has been suggested (Brenner et al., Curr. Op. Struct. Biol., 7(3): 369-376 (1997)) that there are a limited number of folds in a given polypeptide or protein and that once a critical number of structures have been resolved, structural prediction will gain dramatically in accuracy.

Additional methods of predicting secondary structure include “threading” (Jones, D., Curr. Opin. Struct. Biol., 7(3): 377-87 (1997); Sippl et al., Structure, 4(1): 15-9 (1996)), “profile analysis” (Bowie et al., Science, 253: 164-170 (1991); Gribskov et al., Meth. Enzym., 183: 146-159 (1990); Gribskov et al., Proc. Nat. Acad. Sci., 84(13): 4355-8 (1987)), and “evolutionary linkage” (See Home, supra, and Brenner, supra).

Vehicles.

This invention requires the presence of at least one vehicle (F¹) attached to a peptide through the C-terminus or a sidechain of one of the amino acid residues. Multiple vehicles may also be used; e.g., an Fc at the C-terminus and a PEG group at a sidechain.

Fc domain.

An Fc domain is the preferred vehicle. The Fc domain may be fused to the C terminus of the peptides.

As noted above, Fc variants are suitable vehicles within the scope of this invention. A native Fc may be extensively modified to form an Fc variant in accordance with this invention, provided binding to the salvage receptor is maintained; see, for example WO 97/34631 and WO 96/32478. In such Fc variants, one may remove one or more sites of a native Fc that provide structural features or functional activity not required by the fusion molecules of this invention. One may remove these sites by, for example, substituting or deleting residues, inserting residues into the site, or truncating portions containing the site. The inserted or substituted residues may also be altered amino acids, such as peptidomimetics or D-amino acids. Fc variants may be desirable for a number of reasons, several of which are described below. Exemplary Fc variants include molecules and sequences in which:
1. Sites involved in disulfide bond formation are removed. Such removal may avoid reaction with other cysteine-containing proteins present in the host cell used to produce the molecules of the invention. For this purpose, the cysteine-containing segment at the N-terminus may be truncated or cysteine residues may be deleted or substituted with other amino acids (e.g., alanyl, seryl). In particular, one may truncate the N-terminal 20-amino acid segment of SEQ ID NO: 2 or delete or substitute the cysteine residues at positions 7 and 10 of SEQ ID NO: 2. Even when cysteine residues are removed, the single chain Fc domains can still form a dimeric Fc domain that is held together non-covalently.
2. A native Fc is modified to make it more compatible with a selected host cell. For example, one may remove the PA sequence near the N-terminus of a typical native Fc, which may be recognized by a digestive enzyme in E. coli such as proline iminopeptidase. One may also add an N-terminal methionine residue, especially when the molecule is expressed recombinantly in a bacterial cell such as E. coli. The Fc domain of SEQ ID NO: 2 is one such Fc variant.
3. A portion of the N-terminus of a native Fc is removed to prevent N-terminal heterogeneity when expressed in a selected host cell. For this purpose, one may delete any of the first 20 amino acid residues at the N-terminus, particularly those at positions 1, 2, 3, 4 and 5.
4. One or more glycosylation sites are removed. Residues that are typically glycosylated (e.g., asparagine) may confer cytolytic response. Such residues may be deleted or substituted with unglycosylated residues (e.g., alanine).
5. Sites involved in interaction with complement, such as the Clq binding site, are removed. For example, one may delete or substitute the EKK sequence of human IgG1. Complement recruitment may not be advantageous for the molecules of this invention and so may be avoided with such an Fc variant.
6. Sites are removed that affect binding to Fc receptors other than a salvage receptor. A native Fc may have sites for interaction with certain white blood cells that are not required for the fusion molecules of the present invention and so may be removed.
7. The ADCC site is removed. ADCC sites are known in the art; see, for example, Molec. Immunol. 29 (5): 633-9 (1992) with regard to ADCC sites in IgG1. These sites, as well, are not required for the fusion molecules of the present invention and so may be removed.
8. When the native Fc is derived from a non-human antibody, the native Fc may be humanized. Typically, to humanize a native Fc, one will substitute selected residues in the non-human native Fc with residues that are normally found in human native Fc. Techniques for antibody humanization are well known in the art.

Preferred Fc variants include the following. In SEQ ID NO: 2 (FIG. 4) the leucine at position 15 may be substituted with glutamate; the glutamate at position 99, with alanine; and the lysines at positions 101 and 103, with alanines. In addition, one or more tyrosine residues can be replaced by phenyalanine residues.

An alternative vehicle would be a protein, polypeptide, peptide, antibody, antibody fragment, or small molecule (e.g., a peptidomimetic compound) capable of binding to a salvage receptor. For example, one could use as a vehicle a polypeptide as described in U.S. Pat. No. 5,739,277, issued Apr. 14, 1998 to Presta et al. Peptides could also be selected by phage display or RNA-peptide screening for binding to the FcRn salvage receptor. Such salvage receptor-binding compounds are also included within the meaning of “vehicle” and are within the scope of this invention. Such vehicles should be selected for increased half-life (e.g., by avoiding sequences recognized by proteases) and decreased immunogenicity (e.g., by favoring non-immunogenic sequences, as discovered in antibody humanization).

Polymer Vehicles.

As noted above, polymer vehicles may also be used for F¹ and F². Various means for attaching chemical moieties useful as vehicles are currently available, see, e.g., Patent Cooperation Treaty (“PCT”) International Publication No. WO 96/11953, entitled “N-Terminally Chemically Modified Protein Compositions and Methods,” herein incorporated by reference in its entirety. This PCT publication discloses, among other things, the selective attachment of water soluble polymers to the N-terminus of proteins.

A preferred polymer vehicle is polyethylene glycol (PEG). The chemical modification of therapeutic proteins with polyethylene glycol (PEG) has been broadly applied to improve the in vivo efficacy of protein drugs. See “Protein Conjugates” chapter in Harris, et al., American Chemical Society pp. 118-216 (1997). PEGylation achieves this effect by extending the drug's circulating half-life, increasing its solubility and in some cases, reducing the drug's toxicity and immunogenicity.

The PEG group may be of any convenient molecular weight and may be linear or branched. The average molecular weight of the PEG will preferably range from about 2 kiloDalton (“kD”) to about 100 kD. The PEG group may also be attached to more than one therapeutic molecule; for example, in a peptide-PEG-peptide configuration. Two PEG molecules may also be attached to multiple sites or to a single site of a therapeutic molecule; for example, two PEG molecules attached to a cysteine sidechain through a linker. The average molecular weight of the PEG will preferably range from about 2 kiloDalton (“kD”) to about 100 kDa, more preferably from about 5 kDa to about 50 kDa, most preferably from about 5 kDa, 20 kDa, or 30 kDa. In the present invention, linear monomethoxy PEGmaleimides of molecular weights in the range of 5-30 kDa are preferred, with 20-30 kDa polymers most preferred. Also preferred is a 40 kDa branched PEG-maleimide, comprised of two 20 kDa polymer “arms” joined through a linker at the peptide attachment site. Another preferred embodiment employs an 8 kDa bis-functional PEG-(maleimide)₂ which can be used to generate a peptide-PEG-peptide configuration.

A variety of conjugation chemistries have been investigated for coupling PEG non-specifically to proteins. Zalipsky, Advanced Drug Delivery Reviews 16:157-182 (1995). The PEG groups can generally be attached to the compounds of the invention via acylation or reductive alkylation through a reactive group on the PEG moiety (e.g., an aldehyde, amino, thiol, or ester group) to a reactive group on the inventive compound (e.g., an aldehyde, amino, or ester group).

A useful strategy for the PEGylation of synthetic peptides consists of combining, through forming a conjugate linkage in solution, a peptide and a PEG moiety, each bearing a special functionality that is mutually reactive toward the other. The peptides can be easily prepared with conventional solid phase synthesis. The peptides are “preactivated” with an appropriate functional group at a specific site. The precursors are purified and fully characterized prior to reacting with the PEG moiety. Ligation of the peptide with PEG usually takes place in aqueous phase and can be easily monitored by reverse phase analytical HPLC. The PEGylated peptides can be easily purified by preparative HPLC and characterized by analytical HPLC, amino acid analysis and laser desorption mass spectrometry.

Solid phase synthesis is also useful to prepare molecules PEGylated through an available amino group (e.g., a lysine sidechain) using an orthogonal protection strategy. In such synthesis, the peptide is synthesized with removable protecting groups (e.g., Dde for lysine sidechains) attached to the amino groups and any other reactive groups that are not selected for PEGylation. The synthesized peptide can then undergo a reaction resulting in PEGylation of the unprotected amino group. The protecting groups on the other reactive groups can then be removed by conventional means. This technique is especially useful for PEGylation of one of the lysine residues of the PTH fragments mentioned herein. For PTH(1-34), for example, the sidechain of one of the lysine residues at positions 13, 26, or 27 is left unprotected while the others comprise a Dde protecting group. The Dde groups are selectively removed using 2% hydrazine in water for 5 to 30 minutes at room temperature. One may use this technique to prepare a preferred molecule that is PEGylated through the sidechain of the lysine residue at position 27.

Solid phase synthesis techniques may also be employed to prepare molecules having a PEG moiety at the C-terminus. The molecule may comprise a PEG moiety linking it to a resin used for solid phase synthesis. The synthesized molecule may then be cleaved from the resin such that the PEG moiety is retained with the peptide.

Site-directed approaches may be useful to maximize retention of biological activity while minimizing conjugate heterogeneity. Site-directed PEGylation is typically achieved through a combination of recombinant protein techniques and selective conjugation chemistries. First, site-directed mutagenesis is used to introduce unique amino acids with reactive functional groups into the polypeptide sequence at positions predicted to have minimal impact on protein activity Goodson, et al, Bio/Technology 8:343-346 (1990) and Tsutsumi, et al., Proc. Natl. Acad. Sci. 97:8548-8553 (2000). Cysteine is the preferred residue for engineering directed conjugation sites because it is relatively scarce in proteins and the thiol sidechain is among the most reactive of the protein nucleophiles. Next, the mutagenic nucleic acid is introduced into a vector, which then is used to transfect a host cell (e.g., E. coli, which is preferred), and the peptide is expressed and isolated from the host cell. For the PEG portion of the molecule, an activated monofunctional PEG polymer is prepared or obtained commercially. Although there are a wide variety of activated PEG polymers available which react specifically with cysteine thiols, such as PEG-maleimide, -vinylsulfone, -iodoacetamide, -orthopyridyl-disulphide and -epoxides, among others, the PEG-maleimides are by far the most commonly used for conjugation. Zalipsky et al., pp. 347-3⁷0 (1992). Finally, the cysteine-containing protein and activated PEG are combined under appropriate reaction conditions to promote formation of a PEG-protein conjugate which is subsequently purified and characterized. PEG-maleimides are preferred PEGylating reagents, but PEGylation may also be achieved with PEG-vinylsulfones, PEG-orthopyridyl-disulphides and PEG-iodoacetamides or any other activated PEG that is selective for cysteine thiols.

Although cysteine residues are convenient substrates for PEGylation chemistry, as noted above, the PTH-(1-34) peptide and other peptides useful in this invention (e.g., see Tables 1A and 2) contains no natural Cys residues for PEG conjugation. Cys mutations can be introduced at any position, but structure-activity data suggests that C-terminal domain insertions or substitutions are preferred. In one preferred embodiment, cysteine residues were introduced into PTH-(1-34) by substituting Cys for Lysine at position 27, and/or by inserting Cys between histidine and asparagine at position 33.

Polysaccharide polymers are another type of water soluble polymer which may be used for protein modification and may be prepared by techniques generally as described above. Dextrans are polysaccharide polymers comprised of individual subunits of glucose predominantly linked by α1-6 linkages. The dextran itself is available in many molecular weight ranges, and is readily available in molecular weights from about 1 kD to about 70 kD. Dextran is a suitable water soluble polymer for use in the present invention as a vehicle by itself or in combination with another vehicle (e.g., Fc). See, for example, WO 96/11953 and WO 96/05309. The use of dextran conjugated to therapeutic or diagnostic immunoglobulins has been reported; see, for example, European Patent Publication No. 0 315 456, which is hereby incorporated by reference in its entirety. Dextran of about 1 kD to about 20 kD is preferred when dextran is used as a vehicle in accordance with the present invention.

Linkers

Any “linker” group is optional. When present, its chemical structure is not critical, since it serves primarily as a spacer. The linker is preferably made up of amino acids linked together by peptide bonds. Thus, in preferred embodiments, the linker is made up of from 1 to 20 amino acids linked by peptide bonds, wherein the amino acids are selected from the 20 naturally occurring amino acids. Some of these amino acids may be glycosylated, as is well understood by those in the art. In a more preferred embodiment, the 1 to 20 amino acids are selected from glycine, alanine, proline, asparagine, glutamine, and lysine. Even more preferably, a linker is made up of a majority of amino acids that are sterically unhindered, such as glycine and alanine. Thus, preferred linkers are polyglycines (particularly (Gly)₄, (Gly)₅), poly(Gly-Ala), and polyalanines. Other specific examples of linkers are:

| | |
|---|---|
| (Gly)₃Lys(Gly)₄; | (SEQ ID NO: 6) |
| (Gly)₃AsnGlySer(Gly)₂; | (SEQ ID NO: 7) |
| (Gly)₃Cys(Gly)₄; | (SEQ ID NO: 8) |
| and | |
| GlyProAsnGlyGly. | (SEQ ID NO: 9) |

To explain the above nomenclature, for example, (Gly)₃Lys(Gly)₄ means Gly-Gly-Gly-Lys-Gly-Gly-Gly-Gly. Combinations of Gly and Ala are also preferred. The linkers shown here are exemplary; linkers within the scope of this invention may be much longer and may include other residues.

Non-peptide linkers are also possible. For example, alkyl linkers such as —NH—(CH₂)₅—C(O)—, wherein s=2-20 could be used. These alkyl linkers may further be substituted by any non-sterically hindering group such as lower alkyl (e.g., C₁-C₆) lower acyl, halogen (e.g., Cl, Br), CN, NH₂, phenyl, etc. An exemplary non-peptide linker is a PEG linker, VI 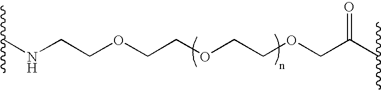
wherein n is such that the linker has a molecular weight of 100 to 5000 kD, preferably 100 to 500 kD. The peptide linkers may be altered to form derivatives in the same manner as described above.

Derivatives

The inventors also contemplate derivatizing the peptide and/or vehicle portion of the compounds. Such derivatives may improve the solubility, absorption, biological half life, and the like of the compounds. The moieties may alternatively eliminate or attenuate any undesirable side-effect of the compounds and the like. Exemplary derivatives include compounds in which:
1. The compound or some portion thereof is cyclic. For example, the peptide portion may be modified to contain two or more Cys residues (e.g., in the linker), which could cyclize by disulfide bond formation.
2. The compound is cross-linked or is rendered capable of cross-linking between molecules. For example, the peptide portion may be modified to contain one Cys residue and thereby be able to form an intermolecular disulfide bond with a like molecule. The compound may also be cross-linked through its C-terminus, as in the molecule shown below. V 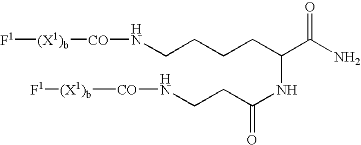
3. One or more peptidyl [—C(O)NR—] linkages (bonds) is replaced by a non-peptidyl linkage. Exemplary non-peptidyl linkages are —CH₂— carbamate [—CH₂—OC(O)NR—], phosphonate, —CH₂-sulfonamide [—CH₂—S(O)₂NR—], urea [—NHC(O)NH—], —CH₂-secondary amine, and alkylated peptide [—C(O)NR⁶— wherein R⁶ is lower alkyl].
4. The N-terminus is derivatized. Typically, the N-terminus may be acylated or modified to a substituted amine. Exemplary N-terminal derivative groups include —NRR¹ (other than —NH₂), —NRC(O)R¹, —NRC(O)OR¹, —NRS(O)₂R¹, —NHC(O)NHR¹, succinimide, or benzyloxycarbonyl-NH— (CBZ—NH—), wherein R and R¹ are each independently hydrogen or lower alkyl and wherein the phenyl ring may be substituted with 1 to 3 substituents selected from the group consisting of C₁-C₄ alkyl, C₁-C₄ alkoxy, chloro, and bromo.
5. The free C-terminus is derivatized. Typically, the C-terminus is esterified or amidated. Exemplary C-terminal derivative groups include, for example, —C(O)R² wherein R² is lower alkoxy or —NR³R⁴ wherein R³ and R⁴ are independently hydrogen or C₁-C₈ alkyl (preferably C₁-C₄ alkyl).
6. A disulfide bond is replaced with another, preferably more stable, cross-linking moiety (e.g., an alkylene). See, e.g., Bhatnagar et al. (1996), J. Med. Chem. 39: 3814-9; Alberts et al. (1993) Thirteenth Am. Pep. Symp., 357-9.
7. One or more individual amino acid residues is modified. Various derivatizing agents are known to react specifically with selected sidechains or terminal residues, as described in detail below.

Lysinyl residues and amino terminal residues may be reacted with succinic or other carboxylic acid anhydrides, which reverse the charge of the lysinyl residues. Other suitable reagents for derivatizing alpha-amino-containing residues include imidoesters such as methyl picolinimidate; pyridoxal phosphate; pyridoxal; chloroborohydride; trinitrobenzenesulfonic acid; O-methylisourea; 2,4 pentanedione; and transaminase-catalyzed reaction with glyoxylate.

Arginyl residues may be modified by reaction with any one or combination of several conventional reagents, including phenylglyoxal, 2,3-butanedione, 1,2-cyclohexanedione, and ninhydrin. Derivatization of arginyl residues requires that the reaction be performed in alkaline conditions because of the high pKa of the guanidine functional group. Furthermore, these reagents may react with the groups of lysine as well as the arginine epsilon-amino group.

Specific modification of tyrosyl residues has been studied extensively, with particular interest in introducing spectral labels into tyrosyl residues by reaction with aromatic diazonium compounds or tetranitromethane. Most commonly, N-acetylimidizole and tetranitromethane are used to form O-acetyl tyrosyl species and 3-nitro derivatives, respectively.

Carboxyl sidechain groups (aspartyl or glutamyl) may be selectively modified by reaction with carbodiimides (R′—N═C═N—R′) such as 1-cyclohexyl-3-(2-morpholinyl-(4-ethyl) carbodiimide or 1-ethyl-3-(4-azonia-4,4-dimethylpentyl) carbodiimide. Furthermore, aspartyl and glutamyl residues may be converted to asparaginyl and glutaminyl residues by reaction with ammonium ions.

Glutaminyl and asparaginyl residues may be deamidated to the corresponding glutamyl and aspartyl residues. Alternatively, these residues are deamidated under mildly acidic conditions. Either form of these residues falls within the scope of this invention.

Cysteinyl residues can be replaced by amino acid residues or other moieties either to eliminate disulfide bonding or, conversely, to stabilize cross-linking. See, e.g., Bhatnagar et al. (1996), J. Med. Chem. 39: 3814-9.

Derivatization with bifunctional agents is useful for cross-linking the peptides or their functional derivatives to a water-insoluble support matrix or to other macromolecular vehicles. Commonly used cross-linking agents include, e.g., 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis(succinimidylpropionate), and bifunctional maleimides such as bis-N-maleimido-1,8-octane. Derivatizing agents such as methyl-3-[(p-azidophenyl)dithio]propioimidate yield photoactivatable intermediates that are capable of forming crosslinks in the presence of light. Alternatively, reactive water-insoluble matrices such as cyanogen bromide-activated carbohydrates and the reactive substrates described in U.S. Pat. Nos. 3,969,287; 3,691,016; 4,195,128; 4,247,642; 4,229,537; and 4,330,440 are employed for protein immobilization.

Carbohydrate (oligosaccharide) groups may conveniently be attached to sites that are known to be glycosylation sites in proteins. Generally, 0-linked oligosaccharides are attached to serine (Ser) or threonine (Thr) residues while N-linked oligosaccharides are attached to asparagine (Asn) residues when they are part of the sequence Asn-X-Ser/Thr, where X can be any amino acid except proline. X is preferably one of the 19 naturally occurring amino acids other than proline. The structures of N-linked and O-linked oligosaccharides and the sugar residues found in each type are different. One type of sugar that is commonly found on both is N-acetylneuraminic acid (referred to as sialic acid). Sialic acid is usually the terminal residue of both N-linked and O-linked oligosaccharides and, by virtue of its negative charge, may confer acidic properties to the glycosylated compound. Such site(s) may be incorporated in the linker of the compounds of this invention and are preferably glycosylated by a cell during recombinant production of the polypeptide compounds (e.g., in mammalian cells such as CHO, BHK, COS). However, such sites may further be glycosylated by synthetic or semi-synthetic procedures known in the art.

Other possible modifications include hydroxylation of proline and lysine, phosphorylation of hydroxyl groups of seryl or threonyl residues, oxidation of the sulfur atom in Cys, methylation of the alpha-amino groups of lysine, arginine, and histidine side chains. Creighton, Proteins: Structure and Molecule Properties (W. H. Freeman & Co., San Francisco), pp. 79-86 (1983).

Compounds of the present invention may be changed at the DNA level, as well. The DNA sequence of any portion of the compound may be changed to codons more compatible with the chosen host cell. For E. coli, which is the preferred host cell, optimized codons are known in the art. Codons may be substituted to eliminate restriction sites or to include silent restriction sites, which may aid in processing of the DNA in the selected host cell. The vehicle, linker and peptide DNA sequences may be modified to include any of the foregoing sequence changes.

Methods of Making

The compounds of this invention largely may be made in transformed host cells using recombinant DNA techniques. To do so, a recombinant DNA molecule coding for the peptide is prepared. Methods of preparing such DNA molecules are well known in the art. For instance, sequences coding for the peptides could be excised from DNA using suitable restriction enzymes. Alternatively, the DNA molecule could be synthesized using chemical synthesis techniques, such as the phosphoramidate method. Also, a combination of these techniques could be used.

The invention also includes a vector capable of expressing the peptides in an appropriate host. The vector comprises the DNA molecule that codes for the peptides operatively linked to appropriate expression control sequences. Methods of effecting this operative linking, either before or after the DNA molecule is inserted into the vector, are well known. Expression control sequences include promoters, activators, enhancers, operators, ribosomal binding sites, start signals, stop signals, cap signals, polyadenylation signals, and other signals involved with the control of transcription or translation.

The resulting vector having the DNA molecule thereon is used to transform an appropriate host. This transformation may be performed using methods well known in the art.

Any of a large number of available and well-known host cells may be used in the practice of this invention. The selection of a particular host is dependent upon a number of factors recognized by the art. These include, for example, compatibility with the chosen expression vector, toxicity of the peptides encoded by the DNA molecule, rate of transformation, ease of recovery of the peptides, expression characteristics, bio-safety and costs. A balance of these factors must be struck with the understanding that not all hosts may be equally effective for the expression of a particular DNA sequence. Within these general guidelines, useful microbial hosts include bacteria (such as E. coli sp.), yeast (such as Saccharomyces sp.) and other fungi, insects, plants, mammalian (including human) cells in culture, or other hosts known in the art.

Next, the transformed host is cultured and purified. Host cells may be cultured under conventional fermentation conditions so that the desired compounds are expressed. Such fermentation conditions are well known in the art. Finally, the peptides are purified from culture by methods well known in the art.

The compounds may also be made by synthetic methods. For example, solid phase synthesis techniques may be used. Suitable techniques are well known in the art, and include those described in Merrifield (1973), Chem. Polypeptides, pp. 335-61 (Katsoyannis and Panayotis eds.); Merrifield (1963), J. Am. Chem. Soc. 85: 2149; Davis et al. (1985), Biochem. Intl. 10: 394-414; Stewart and Young (1969), Solid Phase Peptide Synthesis; U.S. Pat. No. 3,941,763; Finn et al. (1976), The Proteins (3rd ed.) 2: 105-253; and Erickson et al. (1976), The Proteins (3rd ed.) 2: 257-527. Solid phase synthesis is the preferred technique of making individual peptides since it is the most cost-effective method of making small peptides.

Compounds that contain derivatized peptides or which contain non-peptide groups may be synthesized by well-known organic chemistry techniques.

Uses of the Compounds

The compounds of this invention have pharmacologic activity resulting from their interaction with PTH-1 receptor or PTH-2 receptor. Mannstadt et al. (1999), Am. J. Physiol. 277. 5Pt 2. F665-75. PTH and agonists thereof increase bone resorption, increase renal calcium reabsorption, decrease epidermal proliferation, and decrease hair growth. Holick et al. (1994) Proc. Natl. Sci. USA 91 (17): 8014-6; Schilli et al. (1997), J. Invest. Dermatol. 108(6): 928-32. Thus, antagonists of PTH-1 receptor and/or PTH-2 receptor are useful in treating:
primary and secondary hyperparathyroidism;
hypercalcemia, including hypercalcemia resulting from solid tumors (breast, lung and kidney) and hematologic malignacies (multiple myeloma, lymphoma and leukemia); idiopathic hypercalcemia, and hypercalcemia associated with hyperthyroidism and renal function disorders;
tumor metastases, particularly metastases to bone, and particularly related to breast and prostate cancer;
cachexia and anorexia, particularly as associated with cancer;
osteopenia that is related to or aggravated by aberrant PTH receptor signaling, including various forms of osteoporosis, such as:
   primary osteoporosis;
   post-menopausal and age-related osteoporosis;
   endocrine osteoporosis (hyperthyroidism, hyperparathyroidism, Cushing's syndrome, and acromegaly);
   hereditary and congenital forms of osteoporosis (e.g., osteogenesis imperfecta, homocystinuria, Menkes' syndrome, and Riley-Day syndrome);
   osteoporosis due to immobilization of extremities;
   osteoporosis secondary to other disorders, such as hemochromatosis, hyperprolactinemia, anorexia nervosa, thyrotoxicosis, diabetes mellitus, celiac disease, inflammatory bowel disease, primary biliary cirrhosis, rheumatoid arthritis, ankylosing spondylitis, multiple myeloma, lymphoproliferative diseases, and systemic mastocytosis;
   osteoporosis secondary to surgery (e.g., gastrectomy) or to drug therapy, such as chemotherapy, anticonvulsant therapy, immunosuppressive therapy, and anticoagulant therapy;
   osteoporosis secondary to glucocorticosteroid treatment for such diseases as rheumatoid arthritis (RA), systemic lupus erythematosus (SLE), asthma, temporal arteritis, vasculitis, chronic obstructive pulmonary disease, polymyalgia rheumatica, polymyositis, chronic interstitial lung disease;
   osteoporosis secondary to glucocorticosteroid and/or immunomodulatory treatment to prevent organ rejection following organ transplant such as kidney, liver, lung, heart transplants;
   osteoporosis due to submission to microgravity, such as observed during space travel;
   osteoporosis associated with malignant disease, such as breast cancer, prostate cancer;
Paget's disease of bone (osteitis deformans) in adults and juveniles;
osteomyelitis, or an infectious lesion in bone, leading to bone loss;
osteopenia following surgery, induced by steroid administration, and associated with disorders of the small and large intestine and with chronic hepatic and renal diseases.
Osteonecrosis, or bone cell death, associated with traumatic injury or nontraumatic necrosis associated with Gaucher's disease, sickle cell anemia, systemic lupus erythematosus, rheumatoid arthritis, periodontal disease, osteolytic metastasis, and other conditions;
alopecia (deficient hair growth or partial or complete hair loss), including androgenic alopecia (male pattern baldness), toxic alopecia, alopecia senilis, alopecia areata, alopecia pelada, and trichotillomania;
   and the like.

There are other conditions wherein a patient would benefit from the activity of PTH or PTHrP. For those indications, PTH receptor agonists are useful as a therapeutic treatment. In particular, such indications include fracture repair (including healing of non-union fractures), osteopenia, including various forms of osteoporosis, such as:
primary osteoporosis;
post-menopausal and age-related osteoporosis;
endocrine osteoporosis (hyperthyroidism, Cushing's syndrome, and acromegaly);
hereditary and congenital forms of osteoporosis (e.g., osteogenesis imperfecta, homocystinuria, Menkes' syndrome, and Riley-Day syndrome);
osteoporosis due to immobilization of extremities;
osteoporosis secondary to other disorders, such as hemochromatosis, hyperprolactinemia, anorexia nervosa, thyrotoxicosis, diabetes mellitus, celiac disease, inflammatory bowel disease, primary biliary cirrhosis, rheumatoid arthritis, ankylosing spondylitis, multiple myeloma, lymphoproliferative diseases, and systemic mastocytosis;
osteoporosis secondary to surgery (e.g., gastrectomy) or to drug therapy, such as chemotherapy, anticonvulsant therapy, immunosuppressive therapy, and anticoagulant therapy;
osteoporosis secondary to glucocorticosteroid treatment for diseases such as RA, SLE, asthma, temporal arteritis, vasculitis, chronic obstructive pulmonary disease, polymyalgia rheumatica, polymyositis, chronic interstitial lung disease;
osteoporosis secondary to glucocorticosteroid and/or immunomodulatory treatment to prevent organ rejection following organ transplant such as kidney, liver, lung, heart transplants;
osteoporosis due to submission to microgravity, such as observed during space travel;
osteoporosis associated with malignant disease, such as breast cancer, prostate cancer;
   PTH agonists with extended half-life (e. g., those linked to Fc domains) may be used with an inhibitor of bone resorption. Inhibitors of bone resorption include OPG and OPG derivatives, OPG-L (RANKL) antibody, calcitonin (e.g., Miacalcin®, Calcimar®), bisphosphonates (e.g., APD, alendronate, risedronate, etidronate, pamidronate, tiludronate, clodronate, neridronate, ibandronate, zoledronate), estrogens (e.g., Premarin®, Estraderm®, Prempro®, Alora®, Climara®, Vivelle®, Estratab® Ogen®), selective estrogen receptor modulators (e.g., raloxifene, droloxifene, lasofoxifene), tibolone, and the like. Exemplary bone resorption inhibitors are described in WO98/46751 and WO97/23614, which are hereby incorporated by reference in their entirety.

The compounds of this invention may be appropriate as a monotherapy for the treatment of Osteoporosis, and it is possible that the addition of an antiresorptive agent to PTH-Fc treatment will increase both their efficacy and therapeutic window. Both PTH and PTH-Fc cause an increase in both bone formation and bone resorption. The ability of antiresorptives to block the osteoclast response could limit the hypercalcemic effects of PTH-Fc and could also increase bone mas

Pharmaceutical Compositions

In General.

The present invention also provides methods of using pharmaceutical compositions of the inventive compounds. Such pharmaceutical compositions may be for administration for injection, or for oral, pulmonary, nasal, transdermal or other forms of administration. In general, the invention encompasses pharmaceutical compositions comprising effective amounts of a compound of the invention together with pharmaceutically acceptable diluents, preservatives, solubilizers, emulsifiers, adjuvants and/or carriers. Such compositions include diluents of various buffer content (e.g., Tris-HCl, acetate, phosphate), pH and ionic strength; additives such as detergents and solubilizing agents (e.g., Tween 80, Polysorbate 80), anti-oxidants (e.g., ascorbic acid, sodium metabisulfite), preservatives (e.g., Thimersol, benzyl alcohol) and bulking substances (e.g., lactose, mannitol); incorporation of the material into particulate preparations of polymeric compounds such as polylactic acid, polyglycolic acid, etc. or into liposomes. Hyaluronic acid may also be used, and this may have the effect of promoting sustained duration in the circulation. Such compositions may influence the physical state, stability, rate of in vivo release, and rate of in vivo clearance of the present proteins and derivatives. See, e.g., Remington's Pharmaceutical Sciences, 18th Ed. (1990, Mack Publishing Co., Easton, Pa. 18042) pages 1435-1712 which are herein incorporated by reference in their entirety. The compositions may be prepared in liquid form, or may be in dried powder, such as lyophilized form. Implantable sustained release formulations are also contemplated, as are transdermal formulations.

Twice weekly dosing of the compounds of this invention is superior to daily injection of PTH (1-34) for increasing osteoblast number, bone volume, and bone mineral density in rodents. In adult mice, twice weekly dosing with PTH-(1-34)-Fc caused greater increases in bone density and bone volume compared to daily PTH-(1-34). (See FIG. 10.) In an aged OVX rat model of osteoporosis, twice weekly dosing was able to reverse more than 50% of the bone loss induced by one year of estrogen ablation. The effect seen in the aged rat model was even greater when combined with a bisphosphonate (APD). In rats, a single SC injection of PTH-(1-34)-Fc (340 nmol/kg) caused a hypercalcemic response which persisted for 72 hours (FIG. 8). This duration is concordant with the rate of clearance of PTH-(1-34)-Fc from the serum, and is consistent with an optimal twice-weekly dosing regimen in rats.

The optimal dosing of primates may be less frequent compared to rats or mice. Weekly (or less frequent) dosing may be optimal in primates, based on the observation that the hypercalcemic response of OVX cynomolgus monkeys to a single subcutaneous injection of PTH-(1-34)-Fc (10-34 nmol/kg) persisted for about 168 hours (FIG. 11). This observation suggests that a single subcutaneous dose of PTH-(1-34)-Fc in primates is cleared within about 1 week, which could also represent the maximum dosing frequency required for anabolic effects.

Oral Dosage Forms.

Contemplated for use herein are oral solid dosage forms, which are described generally in Chapter 89 of Remington's Pharmaceutical Sciences (1990), 18th Ed., Mack Publishing Co. Easton Pa. 18042, which is herein incorporated by reference in its entirety. Solid dosage forms include tablets, capsules, pills, troches or lozenges, cachets or pellets. Also, liposomal or proteinoid encapsulation may be used to formulate the present compositions (as, for example, proteinoid microspheres reported in U.S. Pat. No. 4,925,673). Liposomal encapsulation may be used and the liposomes may be derivatized with various polymers (e.g., U.S. Pat. No. 5,013,556). A description of possible solid dosage forms for the therapeutic is given in Chapter 10 of Marshall, K., Modern Pharmaceutics (1979), edited by G. S. Banker and C. T. Rhodes, herein incorporated by reference in its entirety. In general, the formulation will include the inventive compound, and inert ingredients which allow for protection against the stomach environment, and release of the biologically active material in the intestine.

Also specifically contemplated are oral dosage forms of the above inventive compounds. If necessary, the compounds may be chemically modified so that oral delivery is efficacious. Generally, the chemical modification contemplated is the attachment of at least one moiety to the compound molecule itself, where said moiety permits (a) inhibition of proteolysis; and (b) uptake into the blood stream from the stomach or intestine. Also desired is the increase in overall stability of the compound and increase in circulation time in the body. Moieties useful as covalently attached vehicles in this invention may also be used for this purpose. Examples of such moieties include: PEG, copolymers of ethylene glycol and propylene glycol, carboxymethyl cellulose, dextran, polyvinyl alcohol, polyvinyl pyrrolidone and polyproline. See, for example, Abuchowski and Davis, Soluble Polymer-Enzyme Adducts, Enzymes as Drugs (1981), Hocenberg and Roberts, eds., Wiley-Interscience, New York, NY,, pp. 367-83; Newmark, et al. (1982), J. Appl. Biochem. 4:185-9. Other polymers that could be used are poly-1,3-dioxolane and poly-1,3,6-tioxocane. Preferred for pharmaceutical usage, as indicated above, are PEG moieties.

For oral delivery dosage forms, it is also possible to use a salt of a modified aliphatic amino acid, such as sodium N-(8-[2-hydroxybenzoyl] amino) caprylate (SNAC), as a carrier to enhance absorption of the therapeutic compounds of this invention. The clinical efficacy of a heparin formulation using SNAC has been demonstrated in a Phase II trial conducted by Emisphere Technologies. See U.S. Pat. No. 5,792,451, “Oral drug delivery composition and methods”.

The compounds of this invention can be included in the formulation as fine multiparticulates in the form of granules or pellets of particle size about 1 mm. The formulation of the material for capsule administration could also be as a powder, lightly compressed plugs or even as tablets. The therapeutic could be prepared by compression.

Colorants and flavoring agents may all be included. For example, the protein (or derivative) may be formulated (such as by liposome or microsphere encapsulation) and then further contained within an edible product, such as a refrigerated beverage containing colorants and flavoring agents.

One may dilute or increase the volume of the compound of the invention with an inert material. These diluents could include carbohydrates, especially mannitol, α-lactose, anhydrous lactose, cellulose, sucrose, modified dextrans and starch. Certain inorganic salts may also be used as fillers including calcium triphosphate, magnesium carbonate and sodium chloride. Some commercially available diluents are Fast-Flo, Emdex, STA-Rx 1500, Emcompress and Avicell.

Disintegrants may be included in the formulation of the therapeutic into a solid dosage form. Materials used as disintegrants include but are not limited to starch including the commercial disintegrant based on starch, Explotab. Sodium starch glycolate, Amberlite, sodium carboxymethylcellulose, ultramylopectin, sodium alginate, gelatin, orange peel, acid carboxymethyl cellulose, natural sponge and bentonite may all be used. Another form of the disintegrants are the insoluble cationic exchange resins. Powdered gums may be used as disintegrants and as binders and these can include powdered gums such as agar, Karaya or tragacanth. Alginic acid and its sodium salt are also useful as disintegrants.

Binders may be used to hold the therapeutic agent together to form a hard tablet and include materials from natural products such as acacia, tragacanth, starch and gelatin. Others include methyl cellulose (MC), ethyl cellulose (EC) and carboxymethyl cellulose (CMC). Polyvinyl pyrrolidone (PVP) and hydroxypropylmethyl cellulose (HPMC) could both be used in alcoholic solutions to granulate the therapeutic.

An antifrictional agent may be included in the formulation of the therapeutic to prevent sticking during the formulation process. Lubricants may be used as a layer between the therapeutic and the die wall, and these can include but are not limited to; stearic acid including its magnesium and calcium salts, polytetrafluoroethylene (PTFE), liquid paraffin, vegetable oils and waxes. Soluble lubricants may also be used such as sodium lauryl sulfate, magnesium lauryl sulfate, polyethylene glycol of various molecular weights, Carbowax 4000 and 6000.

Glidants that might improve the flow properties of the drug during formulation and to aid rearrangement during compression might be added. The glidants may include starch, talc, pyrogenic silica and hydrated silicoaluminate.

To aid dissolution of the compound of this invention into the aqueous environment a surfactant might be added as a wetting agent. Surfactants may include anionic detergents such as sodium lauryl sulfate, dioctyl sodium sulfosuccinate and dioctyl sodium sulfonate. Cationic detergents might be used and could include benzalkonium chloride or benzethonium chloride. The list of potential nonionic detergents that could be included in the formulation as surfactants are lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. These surfactants could be present in the formulation of the protein or derivative either alone or as a mixture in different ratios.

Additives may also be included in the formulation to enhance uptake of the compound. Additives potentially having this property are for instance the fatty acids oleic acid, linoleic acid and linolenic acid.

Controlled release formulation may be desirable. The compound of this invention could be incorporated into an inert matrix which permits release by either diffusion or leaching mechanisms e.g., gums. Slowly degenerating matrices may also be incorporated into the formulation, e.g., alginates, polysaccharides. Another form of a controlled release of the compounds of this invention is by a method based on the Oros therapeutic system (Alza Corp.), i.e., the drug is enclosed in a semipermeable membrane which allows water to enter and push drug out through a single small opening due to osmotic effects. Some enteric coatings also have a delayed release effect.

Other coatings may be used for the formulation. These include a variety of sugars which could be applied in a coating pan. The therapeutic agent could also be given in a film coated tablet and the materials used in this instance are divided into 2 groups. The first are the nonenteric materials and include methyl cellulose, ethyl cellulose, hydroxyethyl cellulose, methylhydroxy-ethyl cellulose, hydroxypropyl cellulose, hydroxypropyl-methyl cellulose, sodium carboxy-methyl cellulose, providone and the polyethylene glycols. The second group consists of the enteric materials that are commonly esters of phthalic acid.

A mix of materials might be used to provide the optimum film coating. Film coating may be carried out in a pan coater or in a fluidized bed or by compression coating.

Pulmonary Delivery Forms.

Also contemplated herein is pulmonary delivery of the present protein (or derivatives thereof). The protein (or derivative) is delivered to the lungs of a mammal while inhaling and traverses across the lung epithelial lining to the blood stream. (Other reports of this include Adjei et al., Pharma. Res. (1990) 7: 565-9; Adjei et al. (1990), Internatl. J. Pharmaceutics 63: 135-44 (leuprolide acetate); Braquet et al. (1989), J. Cardiovasc. Pharmacol. 13 (suppl.5): s.143-146 (endothelin-1); Hubbard et al. (1989), Annals Int. Med. 3: 206-12 (α1-antitrypsin); Smith et al. (1989), J. Clin. Invest. 84: 1145-6 (α1-proteinase); Oswein et al. (March 1990), “Aerosolization of Proteins”, Proc. Symp. Resp. Drug Delivery II, Keystone, Colo. (recombinant human growth hormone); Debs et al. (1988), J. Immunol. 140: 3482-8 (interferon-γ and tumor necrosis factor α) and Platz et al., U.S. Pat. No. 5,284,656 (granulocyte colony stimulating factor).

Contemplated for use in the practice of this invention are a wide range of mechanical devices designed for pulmonary delivery of therapeutic products, including but not limited to nebulizers, metered dose inhalers, and powder inhalers, all of which are familiar to those skilled in the art. Some specific examples of commercially available devices suitable for the practice of this invention are the Ultravent nebulizer, manufactured by Mallinckrodt, Inc., St. Louis, Mo.; the Acorn II nebulizer, manufactured by Marquest Medical Products, Englewood, Col.; the Ventolin metered dose inhaler, manufactured by Glaxo Inc., Research Triangle Park, N.C.; and the Spinhaler powder inhaler, manufactured by Fisons Corp., Bedford, Mass.

All such devices require the use of formulations suitable for the dispensing of the inventive compound. Typically, each formulation is specific to the type of device employed and may involve the use of an appropriate propellant material, in addition to diluents, adjuvants and/or carriers useful in therapy.

The inventive compound should most advantageously be prepared in particulate form with an average particle size of less than 10 μm (or microns), most preferably 0.5 to 5 μm, for most effective delivery to the distal lung.

Pharmaceutically acceptable carriers include carbohydrates such as trehalose, mannitol, xylitol, sucrose, lactose, and sorbitol. Other ingredients for use in formulations may include DPPC, DOPE, DSPC and DOPC. Natural or synthetic surfactants may be used. PEG may be used (even apart from its use in derivatizing the protein or analog). Dextrans, such as cyclodextran, may be used. Bile salts and other related enhancers may be used. Cellulose and cellulose derivatives may be used. Amino acids may be used, such as use in a buffer formulation.

Also, the use of liposomes, microcapsules or microspheres, inclusion complexes, or other types of carriers is contemplated.

Formulations suitable for use with a nebulizer, either jet or ultrasonic, will typically comprise the inventive compound dissolved in water at a concentration of about 0.1 to 25 mg of biologically active protein per mL of solution. The formulation may also include a buffer and a simple sugar (e.g., for protein stabilization and regulation of osmotic pressure). The nebulizer formulation may also contain a surfactant, to reduce or prevent surface induced aggregation of the protein caused by atomization of the solution in forming the aerosol.

Formulations for use with a metered-dose inhaler device will generally comprise a finely divided powder containing the inventive compound suspended in a propellant with the aid of a surfactant. The propellant may be any conventional material employed for this purpose, such as a chlorofluorocarbons a hydrochlorofluorocarbon, a hydrofluorocarbon, or a hydrocarbon, including trichlorofluoromethane, dichlorodifluoromethane, dichlorotetrafluoroethanol, and 1,1,1,2-tetrafluoroethane, or combinations thereof. Suitable surfactants include sorbitan trioleate and soya lecithin. Oleic acid may also be useful as a surfactant.

Formulations for dispensing from a powder inhaler device will comprise a finely divided dry powder containing the inventive compound and may also include a bulking agent, such as lactose, sorbitol, sucrose, mannitol, trehalose, or xylitol in amounts which facilitate dispersal of the powder from the device, e.g., 50 to 90% by weight of the formulation.

Nasal Delivery Forms.

Nasal delivery of the inventive compound is also contemplated. Nasal delivery allows the passage of the protein to the blood stream directly after administering the therapeutic product to the nose, without the necessity for deposition of the product in the lung. Formulations for nasal delivery include those with dextran or cyclodextran. Delivery via transport across other mucous membranes is also contemplated.

Dosages.

The dosage regimen involved in a method for treating the above-described conditions will be determined by the attending physician, considering various factors which modify the action of drugs, e.g. the age, condition, body weight, sex and diet of the patient, the severity of any infection, time of administration and other clinical factors. Generally, the daily regimen should be in the range of 0.1-1000 micrograms of the inventive compound per kilogram of body weight, preferably 0.1-150 micrograms per kilogram.

Specific Preferred Embodiments

The inventors have determined preferred structures for the preferred peptides listed in Table 4 below. The symbol “A” may be any of the linkers described herein or may simply represent a normal peptide bond (i.e., so that no linker is present). Tandem repeats and linkers are shown separated by dashes for clarity.

TABLE 4

Preferred embodiments

| | | SEQ |
|---|---|---|
| | Peptide | ID |
| Sequence/structure | description | NO: |
| SVSEIQLMHNLGKHLNSMERVEWLRKKLQDVHNF- | PTH | 161 |
| Λ-F¹ | (1-34) | |
| SVSEIQLMHNRGKHLNSMERVEWLRKKLQDVHNF- | (L11R) | 162 |
| Λ-F¹ | PTH | |
| | (1-34) | |
| SVSEIQLMHNKGKHLNSMERVEWLRKKLQDVHNF- | (L11K) | 163 |
| Λ-F¹ | PTH | |
| | (1-34) | |
| SVSEIQLMHNLGKHLNSMRRVEWLRKKLQDVHNF- | (E19R) | 164 |
| Λ-F¹ | PTH | |
| | (1-34) | |
| SVSEIQLMHNLGKHLNSMERVEWLRKKLQDV-Λ-F¹ | PTH | 165 |
| | (1-31) | |
| SVSEIQLMHNLGKHLNSMERVEWLRKKLQD-Λ-F¹ | PTH | 166 |
| | (1-30) | |
| LLHNLGKSIQDLRRRFFLHHLIAEIHTA-Λ-F¹ | (D10N, K11L) | 167 |
| | PTHrP | |
| | (7-34) | |
| SLALADDAAFRERARLLAALERRHWLNSY | TlP39 | 168 |
| MHKLLVLDAP-Λ-F¹ | | |
| PEG | Cys27 | 169 |
| \| | PTH(1-34) | |
| SVSEIQLMHNLGKHLNSMERVEWLRKCLQDVHNF | | |
| PEG | Cys33 | 170 |
| \| | PTH(1-34) | |
| SVSEIQLMHNLGKHLNSMERVEWLRKKLQDVHCNF | | |
| PEG PEG | Cys27,33 | 171 |
| \| \| | PTH(1-34) | |
| SVSEIQLMHNLGKHLNSMERVEWLRKCLQDVHCNF | | |

“F¹” is an Fc domain as defined previously herein or PEG, and “PEG” is a molecule comprising polyethylene glycol as described previously herein (e.g., mPEG, which is preferred), which may comprise any linker to enable attachment of polyethylene glycol known in the art. In addition to those listed in Table 4, the inventors further contemplate heterodimers in which each strand of an Fc dimer is linked to a different peptide sequence; for example, a molecule in which one strand can be described by SEQ ID NO: 166, the other by SEQ ID NO: 170 or an Fc linked with any of the sequences in Tables 1A, 1B, and 2.

All of the compounds of this invention can be prepared by methods described in (WO 00/24782).

The invention will now be further described by the following working examples, which are illustrative rather than limiting.

### EXAMPLE 1

### Bioactivity of an Fc-Conjugated PTH/PTHrP Receptor (PTH-R1) Agonist [PTH-(1-34)-Fc]

Introduction

Parathyroid hormone [PTH-(1-34) or native PTH-(1-84)] causes increased bone formation and increased bone mass when injected daily. This anabolic response was previously thought to require brief exposure to PTH, which is facilitated by the short half-life (less than 1 h) of PTH. Clinically, the anabolic effect of PTH therapy requires daily SC injection, which is a significant barrier to the widespread use of PTH. Less frequent injections of PTH would be clinically desirable and could be achieved by increasing the in vivo half-life of PTH. Short-term (intermittent) exposure to PTH (<1 h/day) stimulates osteoblastic bone formation, while long-term (continuous) exposure (>2 h/day) stimulates osteoclastic bone resorption (Dobnig et al, Endocrinology 138: 4607, 1998). The art suggests that PTH with an extended half-life on its own may increase bone resorption and lead to hypercalcemia. However, it should be possible to prevent PTH-induced osteoclast activity with bone resorption inhibitors. Osteoprotegerin (OPG) may be well suited for this purpose. A single treatment of rats, mice or humans with OPG-Fc causes sustained inhibition of bone resorption, by essentially eradicating the osteoclast population. Co-administration of a potent bone resorption inhibitor, like OPG, may provide greater effect. This regimen would theoretically permit the unopposed stimulation of bone formation by PTH, leading to increased bone mass. It is likely that other bone resorption inhibitors, including bisphosphonates or estrogen, would also inhibit PTH-induced bone resorption and could therefore be used in combination with a long-acting PTH molecule. Towards this goal, we have cloned, expressed and purified human PTH-(1-34)-Fc. Fc conjugation of proteins causes a significant increase in their circulating half life, which may permit injections of PTH-(1-34)-Fc on a schedule similar to or identical to that of OPG-Fc. The benefits of this invention include less frequent injections of PTH, from the current standard of once per day to as infrequently as once per quarter.

Materials, Methods, and Results

Hypercalcemia Assay

We tested the potency and duration of effect of PTH-(1-34)-Fc in a murine hypercalcemia model. Briefly, mice were injected once SC with varying doses of PTH-(1-34) or PTH-(1-34)-Fc, and peripheral blood was collected from the retroorbital sinus for determination of blood ionized calcium. The half-life and the potency of PTH-(1-34)-Fc was greater than that of PTH-(1-34), as evidenced by the sustained hypercalcemic response of mice to the former agent (FIG. 4). Hypercalcemia induced by PTH-(1-34) persisted for 6-24 h, while equimolar doses of PTH-(1-34)-Fc caused more sustained hypercalcemia (48-72 h). This duration of response is consistent with greater half-life of the PTH-(1-34)-Fc construct vs. PTH-(1-34). The potency of PTH-(1-34)-Fc was also significantly greater than that of PTH-(1-34) (FIG. 4). The highest dose of PTH-(1-34)-Fc caused a greater increase in peak ionized calcium levels compared with an equimolar dose of PTH-(1-34). Analysis of the area under the curve (AUC) demonstrated that at the highest dose employed, PTH-(1-34)-Fc caused a 2.6-fold greater hypercalcemic response than did equimolar doses of PTH-(1-34).

Anabolic Assay

Having demonstrated the superior pharmacology and half-life of PTH-(1-34)-Fc over PTH-(1-34), we conducted a pilot study to determine whether PTH-(1-34)-Fc co-treatment with OPG-Fc would increase bone mass. Briefly, 6-month-old male Sprague Dawley (SD) rats were divided into groups of 6. Baseline bone mineral density (BMD) was determined in the third lumbar vertebra (L3) of all rats by dual-energy X-ray absorptiometry (DEXA) (Day 0). Rats were then treated according to the following schedule:

Group 1:

Vehicle controls (PBS, injected SC, Days 1, 3, and 5)

Group 2:

OPG-Fc, single SC injection (1 mg/kg) on Day 1

Group 3:

PTH-(1-34), SC injections on Days 1, 3, and 5, at 20 nMoles PTH/kg/injection. This represents an optimal anabolic PTH regimen.

Group 4:

Same as group 3, but with a single OPG-Fc injection on Day 1.

Group 5:

Single SC injection of PTH-(1-34)-Fc at 60 nMoles/kg, on Day 1. This represents a molar dose which is equivalent to the total dose of PTH-(1-34) received by group 3, but in a single injection.

Group 6:

Same as group 5, but with a single OPG-Fc injection (SC, 1 mg/kg) on Day 1.

DEXA of the lumbar spine was performed again on Day 7 to evaluate changes in BMD. BMD in L3 increased modestly with a single injection of OPGFc, or with 3 injections of PTH-(1-34), compared to PBS-treated rats (FIG. 5). PTH-(1-34)+OPG caused a greater increase in BMD than either OPG or PTH-(1-34) alone. As a monotherapy, a single injection of PTH-(1-34)-Fc failed to increase BMD. However, a single injection of PTH-(1-34)-Fc plus a single injection of OPG-Fc caused a significant increase in BMD (FIG. 5). This result provides proof of principle that a PTH construct with extended circulating half life can be combined with a potent antiresorptive, like OPG-Fc, to create an anabolic skeletal response. The anabolic effect of a single treatment with PTH-(1-34)-Fc plus OPG-Fc was greater than that induced by multiple injections of PTH-(1-34), with or without OPG-Fc co-treatment. In conclusion, maximal gains in BMD can be achieved with infrequent injections of PTH-(1-34)-Fc+OPGFc, which is a superior treatment regimen compared to PTH-(1-34), which must be injected daily or every second day.

FIG. 5 shows the effect of PTH-Fc+OPG-Fc on bone mineral density (BMD) in the third lumbar vertebra (L3). Normal 6 month old male rats were treated with PTH-Fc or PTH or vehicle by a single SC injection. Some rats also received a single SC injection of OPG. BMD was determined 7 days later by DEXA. Data represent means ±SD, n=6 rats/group.

### Example 2

### Bioactivity of an Fc-Conjugated PTH/PTHrP Receptor (PTH-R1) Antagonist ([Asn10,Leu11]PTHrP-(7-34)-Fc).

Introduction

Several disease states are associated with increased circulating levels of PTH or PTHrP. Primary and secondary hyperparathyroidism (PHPT and SHPT, respectively), are associated with increased PTH levels, while humoral hypercalcemia of malignancy (HHM) results in elevated PTHrP levels. Both proteins signal through the common PTH/PTHrP receptor (PTH-R1) to cause increases in bone resorption, renal calcium reabsorption, and renal biosynthesis of vitamin D. While bone resorption inhibitors have variable success in inhibiting osteoclastic bone resorption in these disease states, no therapy currently mitigates the intestinal and renal influence of PTH or PTHrP excess on calcemia. Agents which directly antagonize PTH or PTHrP signaling are therefore likely to have greater efficacy compared to resorption inhibitors.

The most studied antagonists of PTH-R1 signaling are based on amino terminal truncations. PTH-(7-34) peptides are fairly effective PTH-R1 antagonists with very mild agonist activity. Compared to PTH-(7-34), PTHrP-(7-34) peptide has greater affinity for PTH-R1 and as such is a more potent antagonist. However, PTHrP-(7-34) also has greater (but still mild) agonist activity compared to PTH-(7-34) (McKee (1990), Endocrinol. 127: 76). The optimal antagonist may combine the weaker agonism of PTH-(7-34) with the stronger antagonism of PTHrP-(7-34). Nutt et al (1990), Endocrinol. 127: 491, demonstrates that substituting Asn10 and Leu11 of PTH into the PTHrP sequence (replacing Asp10 and Lys11) results in a peptide ([Asn10,Leu11]PTHrP-(7-34)-Fc) with virtually no agonist activity but with very potent antagonist activity.

Like native PTH, all peptide-based PTH-R1 antagonists share the property of very short circulating half-lives (<1 h). Furthermore, the amino-terminal truncations which are required to remove receptor agonism, also significantly reduce the affinity of these peptides for PTH-R1. These properties limit the clinical potential of conventional peptide antagonists. Fc-conjugation of amino-terminally truncated PTH- or PTHrP peptides should significantly increase their circulating half life, such that continuous antagonism of PTH-R1 might be achieved with sufficient exposure to these Fc-antagonists.

Materials, Methods and Results

We have cloned, expressed and purified [Asn10,Leu11]PTHrP-(7-34)-Fc. We tested the ability of this compound to antagonize both acute and chronic hypercalcemia responses in mice. PTHrP-(1-34) was used as a calcemic agent to evaluate the acute effects of [Asn10,Leu11]PTHrP-(7-34)-Fc. Because PTHrP is the principal mediator of HHM, this study also represents a model for hypercalcemia-inducing tumors. Briefly, blood ionized calcium (BIC) was measured at baseline, and mice were then challenged with vehicle (PBS) or with PTHrP-(1-34) (0.5 mg/kg) by SC injection. Mice were then treated once SC with varying doses of [Asn10,Leu11]PTHrP-(7-34)-Fc, or with vehicle (PBS). In vehicle-treated mice challenged with PTHrP-(1-34), a transient hypercalcemic response was observed. The peak calcemic response occurred at 3 h post challenge, and persisted until at least 6 h post challenge. [Asn10,Leu11]PTHrP-(7-34)-Fc at 10 mg/kg caused a more rapid normalization of PTHrP-induced hypercalcemia compared to vehicle treatment. A dose of 30 mg/kg completely blocked the calcemic response to PTHrP-(1-34) (FIG. 6).

In order to test the ability of [Asn10,Leu11]PTHrP-(7-34)-Fc to antagonize more chronic hypercalcemia, we used PTH-(1-34)-Fc as a long-acting calcemic agent. This study also represents a model for primary and secondary hyperparathyroidism, diseases which are characterized by persistent elevation of PTH levels. In vehicle-treated mice, a single SC injection of PTH-(1-34)-Fc (30 mg/kg) caused a robust hypercalcemic response in normal mice, reaching a level of 2.75 mg/dl at 24 h post challenge (vs. normal control value of 1.35). A single SC injection of [Asn10,Leu11]PTHrP-(7-34)-Fc at 10-100 mg/kg caused a significant decrease in the peak hypercalcemic response to PTH-(1-34)-Fc at 24 h (FIG. 6).

In conclusion, we have demonstrated antagonistic activity of [Asn10,Leu11]PTHrP-(7-34)-Fc, in both acute and chronic animal models of hypercalcemia. These models employed calcemic agents based on both PTH and on PTHrP sequences. These data suggest that [Asn10,Leu11]PTHrP-(7-34)-Fc, as well as other Fc-conjugated PTH-R1 antagonists, may be effective treatment options for hyperparathyroidism, HHM, and other diseases associated with aberrant PTH-R1 signaling.

### Example 3

### Osteogenic Properties of FC-Conjugated and Native C-Terminally Truncated PTH Fragments

A. cAMP Assays

We tested the relative ability of PTH-Fc constructs to stimulate cAMP accumulation in rat osteoblast-like ROS 17/2.8 cells. Cultures were treated with the phosphodiesterase inhibitor IBMX to promote the accumulation of cAMP. Cultures were then challenged for 15 minutes with either vehicle (PBS), or various PTH constructs. Dose-dependent cAMP accumulation was demonstrated for all fragments. Non-Fc-conjugated PTH-(1-34) was slightly more potent than PTH-(1-31)-Fc and PTH-(1-30)-Fc (FIG. 7). These data demonstrate that Fc-conjugated PTH fragments maintain the ability to activate the AC pathway in osteoblasts.

B. Mouse Bioassay

We then tested the effects of PTH-(1-34), PTH-(1-34)-Fc, PTH-(1-31)-Fc and PTH-(1-30)-Fc in mice. Four week old male mice were injected on days 0, 5, and 10 with vehicle or with PTH fragments, by SC injection. Peripheral blood was obtained for clinical chemistry at 24, 48, and 72 h. Mice were killed on day 15, vertebrae, tibiae and femurs were harvested for histology and one tibia was collected for bone density measurements (peripheral quantitative computed tomography, pQCT). Clinical chemistry endpoints included total serum calcium, serum alkaline phosphatase (AP, a marker of osteoblast activity), and serum tartrate-resistant acid phosphatase (TRAP, a marker of osteoclast activity). For each animal, the ratio of AP:TRAP was calculated as an index of relative osteoblast activity compared to osteoclast activity. A higher AP:TRAP ratio would indicate a potentially more anabolic agent. The relatively high doses (15-fold greater than optimal anabolic doses) were selected base on previous studies which demonstrated significant changes in clinical chemistry endpoints. It was anticipated that lower doses might be required to demonstrate anabolic effects on bone density, and that antiresorptive co-treatment might also be required to achieve anabolic responses.

The clinical chemistry results are demonstrated in FIG. 8. Serum calcium was not significantly different at 24, 48, or 72 h after injection of 300 nmoles/kg (1.2 mg/kg) of PTH-(1-34). This result is consistent with the short half-life of the non-Fc conjugated peptide, which normally causes a transient (12 h) increase in serum calcium. In contrast, an equimolar dose of PTH-(1-34)-Fc caused a dramatic and sustained increase in serum calcium, which peaked at 24 h. PTH-(1-31)-Fc was a more potent calcemic agent, while PTH-(1-30)-Fc was the least calcemic of the 3 Fc peptides (FIG. 8A). Serum AP (osteoblast marker) was unchanged by PTH-(1-34) administration, but was significantly elevated by 300 nmoles/kg of PTH-(1-34)-Fc and by PTH-(1-31)-Fc at 72 h. PTH-(1-30)-Fc demonstrated the greatest elevation of AP, which peaked 72 h after injection of 1,000 nmoles/kg (FIG. 8B). Serum TRAP (osteoclast marker) was not significantly changed by PTH-(1-34), PTH-(1-34)-Fc, or PTH-(1-30)-Fc, but was dramatically increased by PTH-(1-31)-Fc (FIG. 8C). The calculated AP:TRAP ratios were unchanged by PTH-(1-34), and were increased over time by PTH-(1-34)-Fc. The low dose of PTH-(1-31)-Fc (100 nmoles/kg) increased AP:TRAP, while the high dose (1,000 nmoles/kg) decreased AP:TRAP. The greatest increase in AP:TRAP was realized with PTH-(1-30)-Fc (1,000 nmoles/kg) (FIG. 8D).

The effects of the various PTH constructs on bone mineral density (proximal tibial metaphysis) are demonstrated in FIG. 9. At the end of the 15-day study, PTH-(1-34) (300 nmoles/kg) was observed to have a modest (non-significant) anabolic effect when injected on day 0, day 5 and day 10. PTH-(1-34)-Fc (300 nmoles/kg) had no effect on bone density, nor did PTH-(1-31)-Fc at 100 nmoles/kg. Higher doses of PTH-(1-31)-Fc (300-1,000 nmoles/kg) caused significant hypercalcemia-related toxicity, and bones were not harvested from these animals for pQCT. PTH-(1-30)-Fc caused the greatest increase in bone density. There was an apparent reverse dose-response, where PTH-(1-30)-Fc at 100 nmoles/kg had the greatest effect and at 1,000 nmoles/kg had the least effect, although at all doses BMD was greater than in controls (FIG. 9). The reverse dose-response was consistent with the notion that doses employed (chosen for clinical chemistry endpoints) were 5-50 fold higher than the optimal anabolic doses. Low doses of PTH (or PTH-Fc) which fail to significantly increase serum calcium are optimal for anabolic effects. See Hock, J.M. (1992), T. Bone Min. Res. 7:65-72. In the current study, the treatment regimen with the greatest anabolic effect (PTH-(1-30)-Fc at 100 nmoles/kg) was also the only PTH-Fc treatment which failed to significantly increase serum calcium (FIG. 8A).

These data demonstrate the potential anabolic effects of C-terminally truncated PTH-Fc peptides. The longer half-life conferred by Fc conjugation, combined with the selective stimulation of AC/cAMP by C-terminal truncations, may explain the anabolic effect in the absence of a potent bone resorption inhibitor. It is expected that stepwise C-terminal truncation of PTH-(1-30)-Fc will reveal shorter fragments which maintain or exceed the anabolic profile of PTH-(1-30)-Fc. These fragments may be more selective at stimulating osteoblasts, and may be less calcemic, thus providing a wider therapeutic window for anabolic therapy.

### Example 4

### PTH-Fc Treatment as a Monotherapy

The efficacy of PTH-(1-34)-Fc as a monotherapy was demonstrated in adult mice. Briefly, male BDF1 mice (4 months of age) were treated twice per week by subcutaneous injection with various doses of PTH-(1-34)-Fc or with vehicle (PBS). Other mice were treated daily with SC injections of PTH-(1-34) at a dose of 80 μg/kg/day (20 nmol/kg/day), a treatment regimen which is optimal for increasing bone mass in rodents (M. Gunness-Hey and J. M. Hock, Metab. Bone Dis. & Rel. Res. 5:177-181, 1984). After 3 weeks, mice were sacrificed and tibiae were analyzed for bone mineral density (BMD) via pQCT (FIG. 10).

Total tibial BMD and cancelled BMD were both significantly increased by daily PTH-(1-34) injections compared to vehicle-treated controls (FIG. 1, two-way ANOVA, p<0.05). Twice-weekly injections of PTH-(1-34)-Fc caused dose-dependent increases in both total and cancellous BMD which, at the two highest doses (50 and 150 nmol/kg), were significantly greater than the effects of either vehicle or daily PTH-(1-34). Cortical BMD in the tibia was not significantly enhanced by daily PTH-(1-34) treatments. Twice-weekly PTH-(1-34)-Fc caused a dose-dependent increase in cortical BMD which at the highest dose was signficantly greater than that observed in mice treated with vehicle or with daily PTH-(1-34) (p<0.05).

Twice-weekly PTH-(1-34)-Fc also effectively increased BMD as a monotherapy in aged ovariectomized (OVX) rats. Sprague Dawley rats were OVX'd at 3 months of age and allowed to lose bone for 11 months. Other rats were sham-operated and treated twice per week with vehicle (PBS). OVX rats were treated twice per week with SC injections of either vehicle or the bisphosphonate APD (pamidronate, 0.5 mg/kg), or with PTH-(1-34)-Fc (50 nmol/kg) or with APD +PTH-(1-34)-Fc. BMD was determined weekly via dual energy X-ray absorptiometry (DEXA). Rats were sacrificed after 4 weeks of treatment. At the start of treatment, OVX rats had significant reductions in BMD at all skeletal sites analyzed, compared to vehicle-treated OVX rats (FIG. 11, p<0.05, 2-way ANOVA). APD alone did not significantly increase BMD at any skeletal site compared to vehicle-treated OVX rats. PTH-(1-34)-Fc alone caused a significant increase in BMD at the femoral metaphysis after 4 weeks of treatment (p<0.05). Treatment of OVX rats with PTH+ABD was associated with an earlier significant increase in BMD at this site (3 weeks). The combination of APD+PTH-(1-34)-Fc also caused significant BMD increases at the lumbar vertebrae and at the femoral metaphysis (p<0.05). PTH-(1-34)-Fc alone caused a mild and transient hypercalcemic response which resolved spontaneously after day 10 despite continued treatments. The co-administration of APD completely blocked the calcemic effect of PTH-(1-34)-Fc.

These data suggest that PTH-(1-34)-Fc is an effective anabolic agent when used as a monotherapy in both adult mice and aged OVX rats. We have also demonstrated that the addition of an antiresorptive agent (APD) to PTH-(1-34)-Fc was associated with similar or more rapid increases in BMD in aged OVX rats. Co-administration of APD also blocked the transient hypercalcemic response produced by PTH-(1-34)-Fc, which suggests that the therapeutic index of PTH-(1-34)-Fc could be significantly improved by co-administering an effective antiresorptive agent.

### Example 5

### PEG PTH Analogues

Cys mutations were introduced into PTH(1-34) to provide PEGylation sites. The initial cysteine positions were selected based on structure/function activity data suggesting that the C-terminal domain may be most tolerant to PEGylation. These analogs represent a substitution mutant K27C, an insertion mutant at C33 and the dual mutant combining both cysteine positions:
PTH (1-34) analog (Cys 27) (SEQ ID NO: 188, appearing in Table 1B):
PTH (1-34) analog (Cys 33) (SEQ ID NO: 174, appearing in Table 1B):
PTH (1-34) analog (Cys 27/33) (SEQ ID NO: 172, appearing in Table 1B):

Additional cysteine analogs of PTH (1-34) have also been prepared (see Table 1B).

Chemical Reagents.

All Fmoc-amino acids and Fmoc-Phe-Wang resin was obtained from Midwest Bio-Tech. (Fishers, Ind.). HOBt/DMF and DCC/NMP were purchased from Q Bio-Gene (Carlsbad, Calif.). HBTU was purchased from either Q Bio-Gene (Carlsbad, Calif.) or NovaBiochem (San Diego, Calif.) Bulk solvents: ACN, MeOH, DCM, DMF, and NMP were purchased from EM Science (Gibbstown, N.J.). NEM and piperidine were obtained from Aldrich (Milwaukee, Wis.). Phenol, and TIS were purchased from Sigma (St. Louis, Mo.). TFA was purchased from either PE Applied Biosystems (Foster City, Calif.) or Chem-Impex (Wood Dale, Ill.). All reagents for amino acid analysis were purchased from PE Applied Biosystems (Foster City, Calif.). All chemicals were of the highest grade possible.

Solid-Phase Peptide Synthesis of PTH(1-34) and Analogues.

All peptides were prepared by solid phase synthesis using the Fmoc/t-butyl-based methodology with Fmoc-Phe-Wang or Fmoc-Cyc(Trt)-Wang resin as a solid support. The sidechain protection scheme is as follows: Arg(Pbf), Asn(Trt), Asp(OtBu), Cys(Trt), Gln(Trt), Glu(OtBu), His(Trt), Lys(Boc), Ser(tBu), and Trp(Boc). A PE Applied Biosystems (Foster City, CA) 431 instrument or a Rainin Symphony Multiplex Peptide Synthesizer (Protein Technologies Inc, Woburn, Mass.) with the manufacturer's standard Fmoc program was utilized for solid phase synthesis peptide chain assembly at either 0.05, 0.10, or 0.20 mmol scale. Each coupling consisted of the following modules: (i) removal of the α-amino Fmoc protection by piperidine in NMP (ABI instrument) or NEM in DMF (Rainin instrument); (ii) activation with either HBTU or DCC followed by (iii) single 60-minute coupling of the HOBt ester of the Fmoc amino acid (20, 10, or 5 equivalents) in NMP or DMF; (iv) resin washes.

Upon completion of the automated synthesis, the peptide-resin was dried in vacuo and subjected to acidolytic deprotection and cleavage (TFA:H₂O:TIS:EDT:thioanisol (85:5:5:2.5:2.5, 10 ml, 4 hr, 20° C.). After cleavage and deprotection, the resin/peptide suspension was filtered, peptide-filtrate was precipitated using cold diethyl ether (40 ml), pelleted by centrifugation, washed with cold diethyl ether (2x , 40 ml), and dried in vacuo or by speedvac.

The precipitated crude peptides were analyzed by HPLC-MS for crude purity and expected molecular ion. Analytical HPLC was carried out on a Vydac (Hesperia, Calif.) 214TPTM C18 column (300 Å pore size, 5m particle size, 0.46×25 cm). The conditions for this and all subsequent primary analytical RP-HPLC were linear gradients of 5-50% ACN in 0.1% aqueous TFA over 25 minutes, and a flow rate of 1.0 ml/min, unless otherwise noted. The effluent was monitored with a PDA detector from which the 220 nm absorbance profile was extracted.

Electrospray Mass Spectrometry.

Mass spectra of all peptides were obtained with an PE-Sciex API-I (Thornhill, ON) single quadrupole electrospray mass spectrometer equipped with a nebulizer-assisted electrospray source. Samples of preparative HPLC fractions were introduced by means of an Alltech (Deerfield, Ill.) 426 HPLC pump and Perkin Elmer (Norwalk, Conn.) Series 200 autosampler, using a 0.40 ml/min isocratic gradient of 1 mM NH₄OAc in ACN:H₂0 (50:50). The mass spectrometer was scanned from mass to charge (m/z) 300 to 2400 for the characterization of the purified peptide. The acquisition time was 0.20 minutes, and 10 scans were used for each spectrum. Masses were calculated from the m/z ratios from the observed protonation states of the peptide using Sciex (Thornhill, ON) MacSpec™ software. Theoretical masses were calculated using Sciex (Thornhill, ON) MacBioSpec™ software

Secondary RP-HPLC Analysis.

All peptides were subjected to two additional analytical RP-HPLC protocols to ensure peak homogeneity. Samples were analyzed on a YMC (Wilmington, Calif.) ODS-AQ C18 column (2.0×50 mm). The conditions for this chromatography were a linear gradient of 5 to 50% ACN in 0.1% aqueous TFA over 45 minutes, and a flow rate of 0.4 ml/min. Samples were also analyzed on a Vydac (Hesperia, Calif.) 218TPTM C18 column (300 Å pore size, 5 μm particle size, 0.46×25 cm). The conditions for this analysis were a linear gradient of 5 to 20% ACN in 0.1% aqueous TFA over 10 minutes, followed by 20 to 50% ACN in 0.1% aqueous TFA over 40 minutes, and a flow rate of 1 ml/min. All secondary analytical RP-HPLC were monitored with a PDA detector from which the 220 nm absorbance result was extracted.

Amino Acid Analysis.

Qualitative amino acid analyses were performed on purified peptides. All analyses were performed on an PE Applied Biosystems (Foster City, Calif.) 420A derivatizer with a 130A separation system. The peptides were manually hydrolyzed in vacuo using 6 N HCl at 150° C. for 60 minutes and then applied (approximately 1 μg) to the amino acid analyzer for derivatization as the PTC derivative. The amino acid mixture was then separated by RP-HPLC on a Brownlee PTC C18 column (2.1×220 mm) with a linear gradient of 2% to 11% ACN in H₂0 over 4 minutes, 11% to 27% ACN in H₂0 over 6 minutes, and 27% to 47% ACN in H₂O over 10 minutes. Both solvents were buffered with NaOAc to pH 5.4, and the flow rate was 0.3 ml/min. Quantitative amino acid analyses were performed in triplicate, and composition was determined by comparison of each amino acid peak area in the peptide to the area of a known amount of amino acid standard.

PEGylation.

The PTH (1-34) cysteine analogs were PEGylated with a variety of PEG-maleimides, as shown in Reaction Scheme 1 below.

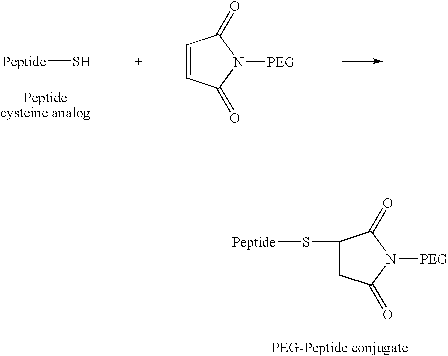

The PEGylation reactions were carried out in 20 mM sodium phosphate, 5 mM EDTA, pH 6.5 with PTH (1-34) peptide concentrations from 2-10 mg/ml and PEG:peptide stoichiometry of 0.5 to 5 fold molar excess. Although reactant concentrations may exceed these limits, the preferred conditions are 5 mg/ml peptide with an equimolar PEG-maleimide concentration. Reaction times may range from 15 minutes to overnight at room temperature or 4 degrees C., with 2 hours at room temperature preferred. Optionally, the reaction may be stopped with the addition of excess mercaptan, such as β-mercaptoethanol. It is further anticipated that PTH (1-34) PEGylation can be achieved by coupling in non-aqueous solvents or through orthogonal approaches during peptide synthesis. The PEG-PTH (1-34) conjugates were then purified by aqueous phase cation exchange chromatography (FIG. 13).

A list of PEGylated PTH peptides appears in Table 5 below

TABLE 5
PEG-PTH peptides tested in vitro and in vivo. The
summarized assay results are relative to either PTH-(1-34) peptide or to
PTH-(1-34)-Fc, as indicated.

| Peptide Name | Description |
|---|---|
| C27 | Peptide Analog, C27 mutant |
| C27-5 K | 5 kD, K27C Series |
| C27-10 K | 10 kD, K27C Series |
| C27-20 K | 20 kD, K27C Series |
| C27-30 K | 30 kD, K27C Series |
| C27-2 X 20 K | 2 X 20 kD branched, K27C Series |
| C27-8 K-C27 | 8 kD Dumbbell, K27C |
| C33 | Peptide Analog, C33 |
| C33-20 K | 20 kD, C33 Series |
| C27/33 | Peptide Analog, C27/33 |
| C27/33-2 X 20 K | 20 kD dual PEG, C27/33 |
| C27/33-2 X 30 K | 30 kD dual, C27/33 |
| C33-30 K | 30 kD, C33 |

In vitro Characterization by cAMP Assay.

PEG-PTH molecules were screened in vitro for evidence of PTH receptor (PTHR1) activation by monitoring production of cAMP in murine MC3T3-E1 osteoblast cultures. PTH-(1-34) peptide consistently demonstrates the greatest potency in this assay, while PTH-(1-34)-Fc is a full agonist with slightly reduced potency. The mutant peptide analogs used for PEGylation (C27, C33 and C27/33) had potencies in the cAMP assay that were similar to each other and to PTH-(1-34)-Fc (FIG. 14A through 14D). In most cases, PEGylation of these peptides had little effect on their relative potency in cAMP assays, and all PEG contructs appeared to behave as full agonists.

In vivo Characterization:

Hypercalcemia assays. PEGPTH molecules were also screened for evidence of sustained PTHR1 activation in young male BDF1 mice. Briefly, mice were injected subcutaneously with PEG-PTH or control constructs and blood ionized calcium (BIC) levels were monitored for up to 120 hours. Increases in BIC reflect increased bone resorption, which is a pharmacologic response to PTH treatment. PTH-(1-34) peptide causes a transient hypercalcemic response in mice which ends within about 6 hours of injection. In contrast, the hypercalcemia induced by equimolar injections with PTH-(1-34)-Fc lasts for approximately 72 hours (FIG. 15). The longer duration of hypercalcemia with PTH-Fc reflects a longer circulating half-life, an attribute which also permits less frequent dosing relative to PTH peptide. As expected, no hypercalcemic responses were evident at 24 hours after injection of the non-PEGylated peptides PTH-(1-34), C27, or C33. This observation is consistent with the predicted short half-life of these peptides.

Other PEG-PTH constructs caused hypercalcemic responses which were similar to those observed with equimolar doses of PTH-(1-34)-Fc, including:
C27-20K
C27-30K
C27-2×20K
C33-20K

The C27-30K construct caused the greatest hypercalcemic response in this assay, and this peptide and C33-30K were then tested in a dose-response hypercalcemia assay. At equimolar doses, C27-30K and C33-30K PEG-PTH molecules were 3-10 fold more potent than PTH-(1-34)-Fc in hypercalcemia assays (FIG. 16). For a given peak level of blood ionized calcium, therapeutic doses of PEG-PTH and PTH-Fc molecules caused similar durations of hypercalcemia. At higher (toxic) doses, PEG-PTH constructs caused a slightly longer duration of hypercalcemia relative to PTH-(1-34)-Fc (FIG. 16).

In vivo Characterization:

Bone anabolic assays. PEG-PTH molecules were also tested for bone anabolic activity in adult male BDF1 mice. The doses were chosen based on the relative potencies of C27-30K PEG-PTH and PTH-(1-34)-Fc in mouse hypercalcemia assays. PTH-(1-34)-Fc at 150 nmol/kg caused a significant increase in tibial trabecular bone mineral density (BMD) when injected twice-weekly for 4 weeks. C27-30K PEG-PTH was similarly anabolic at 3-10 fold lower doses than PTH-(1-34)-Fc when injected twice-weekly (FIG. 17). C27-30K PEG-PTH also caused significant gains in BMD when injected once weekly at the 50 nmol/kg dose.

Discussion and Conclusions.

These data indicate the feasibility of using PEGylation to increase the circulating half-life of PTH peptides. The longer half-life conferred by PEGylation permits infrequent subcutaneous dosing in rodents to obtain bone anabolic effects. The pharmacologic effects of PEG-PTH are similar to those observed with PTH-Fc constructs, with respect to CAMP activation in vitro as well as hypercalcemic and bone anabolic effects in vivo. These similarities in pharmacology suggest that increasing the half-life of PTH peptides, regardless of the means, could represent an important therapeutic advance. Neither Fc nor PEG are absolutely required to realize the therapeutic benefit of a long-acting PTH molecule. These data would also predict that a slow-release formulation of PTH peptides such as those described in Tables 1A, 1B and 2 would achieve the same desired benefit of less frequent injections.

The invention now being fully described, it will be apparent to one of ordinary skill in the art that many changes and modifications can be made thereto, without departing from the spirit and scope of the invention as set forth herein.

Abbreviations

| | |
|---|---|
| aa | amino acid |
| AC | adenylate cyclase |
| Acm | acetamidomethyl |
| ACN | acetonitrile |
| amu | atomic mass unit |
| AP | alkaline phosphatase |
| BMD | bone mineral density |
| Boc | tert-butyloxycarbonyl |
| β-Me | β-mercaptoethanol |
| CAMP | cyclic adenosine monophosphate |
| DCC | Dicyclohexylcarbidiimide |
| DCM | dichloromethane |
| Dde | 1-(4,4-dimethyl-2,6-dioxocyclohex-1-ylidene)ethyl |
| DEXA | dual-energy X-ray absorptiometry |
| DMF | dimethyformamide |
| DTT | dithiothreitol |
| EDT | ethanedithiol |
| ES-MS | electrospray-mass spectrometry |
| Fmoc | fluorenylmethoxycarbonyl |
| HHM | humoral hypercalcemia of malignancy |
| HOBt | 1-hydroxybenzotriazole |
| HBTU | O-(benzotriazol-1-yl)-N,N,N′,N′-tetramethyluronium |
| | hexafluorophosphate; |
| MeOH | methanol |
| NEM | N-ethylmorpholine |
| NH₄Oac | ammonium acetate |
| NMP | N-methylpyrrolidinone |
| OPG | osteoprotegerin |
| OVX | ovariectomized |
| PBS | phosphate-buffered saline |
| Pbf | 2,2,4,6,7-pentamethyldihydrobenzofuran-5-sulfonyl |
| PDA | photodiode array |
| pQCT | peripheral quantitative computed tomography |
| PTC | phenylisothiocyanate; |
| PTH | parathyroid hormone |
| PTHrP | parathyroid hormone-related protein |
| RP-HPLC | reversed-phase high-pressure liquid chromatography |
| tBu | tert-butyl |
| TFA | trifluoroacetic acid |
| TIS | triisopropylsilane |
| TRAP | tartrate-resistant acid phosphatase |
| Trt | trityl (triphenylmethyl) |

1. A composition of matter of the formula
P¹-(L¹)ₐ-F¹
and multimers thereof, wherein:
F¹ is a vehicle and is attached at the C-terminus of P¹-(L¹)ₐ or through a sidechain at any residue from residue 14 through the C-terminal residue;
P¹ is a PTH/PTHrP modulating domain;
L¹ is a linker; and
a is 0 or 1.

2. The composition of matter of claim 1 of the formulae
P¹-F¹.

3. The composition of matter of claim 1, wherein F¹ is a PEG molecule.

4. The composition of matter of claim 1 wherein the PTH/PTHrP modulating domain is of the formula

| | |
|---|---|
| X^{N}HX¹⁰X¹¹X¹²KX¹⁴X¹⁵X¹⁶X¹⁷X¹⁸X¹⁹RX²¹X²²X²³X²⁴X²⁵X²⁶X²⁷X²⁸X^{C} | (SEQ ID NO: 3) |

wherein:
X^{N} is absent or is X³X⁴X⁵X⁶X⁷, X²X³X⁴X⁵X⁶X⁷, X¹X²X³X⁴X⁵X⁶X⁷, or YX¹X²X³X⁴X⁵X⁶X⁷;
X¹ through X⁷, X¹⁰, X¹¹, X¹², X¹⁴ through X²⁸ are each independently amino acid residues;
X^{C} is absent or is X²⁹, X²⁹X³⁰ X²⁹X³⁰X³¹, X²⁹X³⁰X³¹X³², X²⁹X³⁰X³¹X³²X³³, X²⁹X³⁰X³¹X³²X³³X³⁴, X²⁹X³⁰X³¹X³²X³³X³⁴X³⁵, or X²⁹X³⁰X³¹X³²X³³X³⁴X³⁵X³⁶.
X²⁹ through X³⁶ are each independently amino acid residues;
provided that one or more of X¹⁴ through X³⁶ is a cysteine residue.

5. The composition of matter of claim 4, wherein:
X^{N} is X¹X²X³X⁴X⁵X⁶X⁷;
X¹ is a hydrophilic or nonfunctional residue;
X² is V;
X³is S;
X⁴ is E;
X⁵ is a nonfunctional or basic residue;
X⁶ is Q;
X⁷ is L;
X¹⁰ is an acidic or hydrophilic residue;
X¹¹ is a nonfunctional or basic residue;
X¹² is a nonfunctional residue;
X¹⁴ is a basic or hydrophilic residue;
X¹⁵ is a nonfunctional residue;
X¹⁶ is a nonfunctional or hydrophilic residue;
X¹⁷ is an acidic, hydrophilic, or nonfunctional residue;
X¹⁸ is a nonfunctional residue;
X¹⁹ is an acidic or basic residue;
X²¹ is a nonfunctional or basic residue;
X²² is a hydrophilic, acidic, or aromatic residue;
X²³ is an aromatic or lipophilic residue;
X²⁴ is a lipophilic residue (L preferred);
X²⁵ is a hydrophilic or basic residue;
X²⁶ is a hydrophilic or basic residue;
X²⁷ is a lipophilic, basic, or nonfunctional residue; and
X²⁸ is a lipophilic or nonfunctional residue.

6. The composition of matter of claim 5, wherein:
X^{C} is X²⁹X³⁰X³¹X³²X³³X³⁴;
X²⁹ is a hydrophilic or nonfunctional residue;
X³⁰ is a hydrophilic or acidic residue;
X³¹ is a lipophilic or nonfunctional residue;
X³² is H;
X³³ is a cysteine or a hydrophilic residue; and
X³⁴ is a nonfunctional or aromatic residue.

7. The composition of matter of claim 6, wherein:
X^{C} is X²⁹X³⁰X³¹;
X²⁹ is a hydrophilic or nonfunctional residue;
X³⁰ is a hydrophilic or acidic residue; and
X³¹ is a lipophilic or nonfunctional residue.

8. The composition of matter of claim 7, wherein:
X^{C} is X²⁹X^{30;}
X²⁹ is a hydrophilic or nonfunctional residue; and
X³⁰ is a hydrophilic or acidic residue.

9. The composition of matter of claim 7, wherein:
X^{C} is X²⁹; and
X²⁹ is a hydrophilic or nonfunctional residue.

10. The composition of claim 7, wherein at least one of X²⁷ and X³³ is cysteine.

11. The composition of matter of claim 7, wherein X^{C} is absent.

12. The composition of matter of claim 7, wherein:
X¹ is A, S or Y;
X⁵ is H or I;
X¹⁰ is N or D;
X¹¹ is L, R, or K;
X¹² is G, F, or W;
X¹⁴ is H or S;
X¹⁵ is L or I;
X¹⁶ is Q, N, S, or A;
X¹⁷ is S, D, or L;
X¹⁸ is M, L, V or Nle;
X¹⁹ is E or R;
X²¹ is V, M, R, or Nle;
X²² is E or F;
X²³ is W or F;
X²⁵ is R or H;
X²⁶ is K or H;
X²⁷ is C; and
X²⁸ is L or I.

13. The composition of matter of claim 12, wherein:
X^{C} is X²⁹X³⁰X³¹X³²X³³X³⁴;
X²⁹ is Q or A;
X³⁰ is D or E;
X³¹ is V or I;
X³³ is C;
X³⁴ is N or T; and
X³⁵is A, F or Y.

14. The composition of matter of claim 12, wherein:
X^{C} is X²⁹X³⁰X³¹;
X²⁹ is Q or A;
X³⁰ is D or E; and
X³¹ is V or I;

15. The composition of matter of claim 12, wherein:
X^{C} is X²⁹X³⁰;
X²⁹ is Q or A; and
X³⁰ is D or E.

16. The composition of matter of claim 12, wherein:
X^{C} is X²⁹; and^{;}
X²⁹ is Q or A.

17. The composition of matter of claim 12, wherein X^{C} is absent.

18. The composition of matter of claim 1, wherein the PTH/PTHrP modulating domain is of the formula
(SEQ ID NO: 4)
J^{N}J⁷J⁸HNJ¹¹J¹²KHLJ¹⁶SJ¹⁸J¹⁹RJ²¹EWLRKKLJ^{C}
wherein:
J^{N} is absent or is selected from J¹J²J³J⁴J⁵J⁶, J²J³J⁴J⁵J⁶, J³J⁴J⁵J⁶;
J¹ is an amino acid residue;
J² is an amino acid residue;
J³ is an amino acid residue;
J⁴ is an amino acid residue;
J⁵ is an amino acid residue;
J⁶ is an amino acid residue;
J⁷ is an amino acid residue;
J⁸ is an amino acid residue;
J¹¹ is a nonfunctional or basic residue;
J¹² is an amino acid residue;
J¹⁶ is an amino acid residue;
J¹⁸ is an amino acid residue;
J¹⁹ is an acidic or basic residue;
J²¹ is an amino acid residue;
J^{C} is absent or is J²⁹, J²⁹J³⁰, J²⁹J³⁰J³¹, J²⁹J³⁰J³¹J³², J²⁹J³⁰J³¹J³²J³³, J²⁹J³⁰J³¹J³²J³³J³⁴; and
J²⁹ is an amino acid residue;
J³⁰ is an amino acid residue;
J³¹ is an amino acid residue;
J³² is an amino acid residue;
J³³ is an amino acid residue;
J³⁴ is an amino acid residue;
provided that one or more of J¹⁴ through the C-terminal residue is a cysteine residue.

19. The composition of matter of claim 18, wherein:
J^{N} is J¹J²J³J⁴J⁵J⁶;
J¹ is a nonfunctional or aromatic residue;
J² is a nonfunctional residue;
J³ is a hydrophilic residue;
J⁴ is an acidic residue;
J⁵ is a nonfunctional residue;
J⁶ is a basic residue;
J⁷ is a nonfunctional or aromatic residue;
J⁸ is a nonfunctional residue;
J¹¹ is a basic or a nonfunctional residue;
J¹² is a nonfunctional or aromatic residue;
J¹⁶ is a nonfunctional or hydrophilic residue;
J¹⁸ is a nonfunctional residue;
J¹⁹ is an acidic or basic residue; and
J²¹ is a nonfunctional residue;
J^{C} is J²⁹J³⁰J³¹J³²J³³J³⁴;
J²⁹ is a hydrophilic or nonfunctional residue;
J³⁰ is a hydrophilic or acidic residue;
J³¹ is a lipophilic or nonfunctional residue;
J³² is a basic residue;
J³³ is an acidic residue; and
J³⁴ is an aromatic residue.

20. The composition of matter of claim 19, wherein:
J¹ is A, S or Y;
J² is V;
J³ is S;
J⁴ is E;
J⁵ is I;
J⁶ is Q;
J⁷ is L or F;
J⁸ is M or Nle;
J¹¹ is L, R, or K;
J¹² is G or W;
J¹⁶ is N, S, or A;
J¹⁸ is M, Nle, L, or V;
J¹⁹ is E or R;
J²¹ is V, M, or Nle;
J is Q or A;
J³⁰ is D or E;
J³¹ is V or I;
J³² is H;
J³³ is N; and
J³⁴ is F or Y.

21. The composition of matter of claim 18, wherein:
J^{N} is J¹J²J³J⁴J⁵J⁶;
J¹ is a nonfunctional or aromatic residue;
J² is a nonfunctional residue;
J³ is a hydrophilic residue;
J⁴ is an acidic residue;
J⁵ is a nonfunctional residue;
J⁶ is a basic residue;
J⁷ is a nonfunctional or aromatic residue;
J⁸ is a nonfunctional residue;
J¹¹ is a basic or a nonfunctional residue;
J¹² is a nonfunctional or aromatic residue;
J¹⁶ is a nonfunctional or hydrophilic residue;
J¹⁸ is a nonfunctional residue;
J¹⁹ is an acidic or basic residue;
J²¹ is a nonfunctional residue;
J^{C} is J²⁹J³⁰J³¹;
J²⁹ is a hydrophilic or nonfunctional residue;
J³⁰ is a hydrophilic or acidic residue; and
J³¹ is a lipophilic or nonfunctional residue.

22. The composition of matter of claim 21, wherein:
J¹ is A, S or Y;
J² is V;
J³ is S;
J⁴ is E;
J⁵ is I;
J⁶ is Q;
J⁷ is L or F;
J⁸ is M or Nle;
J¹¹ is L, R, or K;
J¹² is G or W;
J¹⁶ is N, S, or A;
J¹⁸ is M, Nle, L, or V;
J¹⁹ is E or R;
J²¹ is V, M, or Nle;
J²⁹ is Q or A;
J³⁰ is D or E; and
J³¹ is V or I.

23. The composition of matter of claim 18, wherein:
J^{N} is J¹J²J³J⁴J⁵J⁶;
J¹ is a nonfunctional or aromatic residue;
J² is a nonfunctional residue;
J³ is a hydrophilic residue;
J⁴ is an acidic residue;
J⁵ is a nonfunctional residue;
J⁶ is a basic residue;
J⁷ is a nonfunctional or aromatic residue;
J⁸is a nonfunctional residue;
J¹¹ is a basic or a nonfunctional residue;
J¹² is a nonfunctional or aromatic residue;
J¹⁶ is a nonfunctional or hydrophilic residue;
J¹⁸ is a nonfunctional residue;
J¹⁹ is an acidic or basic residue;
J²¹ is a nonfunctional residue;
J^{C} is J²⁹J³⁰;
J²⁹ is a hydrophilic or nonfunctional residue; and
J³⁰ is a hydrophilic or acidic residue.

24. The composition of matter of claim 23, wherein:
J is A, S or Y;
J² is V;
J³ is S;
J⁴ is E;
J⁵ is I;
J⁶ is Q;
J⁷ is L or F;
J⁸ is M or Nle;
J¹¹ is L, R, or K;
J¹² is G or W;
J¹⁶ is N, S, or A;
J¹⁸ is M, Nle, L, or V;
J¹⁹ is E or R;
J²¹ is V, M, or Nle;
J²⁹ is Q or A; and
J³⁰ is D or E.

25. The composition of matter of claim 18, wherein:
J^{N} is J¹J²J³J⁴J⁵J⁶;
J¹ is a nonfunctional or aromatic residue;
J² is a nonfunctional residue;
J³ is a hydrophilic residue;
J⁴ is an acidic residue;
J⁵ is a nonfunctional residue;
J⁶ is a basic residue;
J⁷ is a nonfunctional or aromatic residue;
J⁸ is a nonfunctional residue;
J¹¹ is a basic or a nonfunctional residue;
J¹² is a nonfunctional or aromatic residue;
J¹⁶ is a nonfunctional or hydrophilic residue;
J¹⁸ is a nonfunctional residue;
J¹⁹ is an acidic or basic residue;
J²¹ is a nonfunctional residue;
J^{C} is J²⁹; and
J²⁹ is a hydrophilic or nonfunctional residue.

26. The composition of matter of claim 25, wherein:
J¹ is A, S or Y;
J² is V;
J³ is S;
J⁴ is E;
J⁵ is I;
J⁶ is Q;
J⁷ is L or F;
J⁸is M or Nle;
J¹¹ is L, R, or K;
J¹² is G or W;
J¹⁶ is N, S, or A;
J¹⁸ is M, Nle, L, or V;
J¹⁹ is E or R;
J²¹ is V, M, or Nle; and
J²⁹ is Q or A.

27. The composition of matter of claim 18, wherein:
J^{N} is J¹J²J³J⁴J⁵J⁶;
J¹ is a nonfunctional or aromatic residue;
J² is a nonfunctional residue;
J³ is a hydrophilic residue;
J⁴ is an acidic residue;
J⁵ is a nonfunctional residue;
J⁶ is a basic residue;
J⁷ is a nonfunctional or aromatic residue;
J⁸ is a nonfunctional residue;
J¹¹ is a basic or a nonfunctional residue;
J¹² is a nonfunctional or aromatic residue;
J¹⁶ is a nonfunctional or hydrophilic residue;
J¹⁸ is a nonfunctional residue;
J¹⁹ is an acidic or basic residue;
J²¹ is a nonfunctional residue; and
J^{C} is absent.

28. The composition of matter of claim 27, wherein:
J¹ is A, S or Y;
J² is V;
J³ is S;
J⁴ is E;
J⁵ is I;
J⁶ is Q;
J⁷ is L or F;
J⁸ is M or Nle;
J¹¹ is L, R, or K;
J¹² is G or W;
J¹⁶ is N, S, or A;
J¹⁸ is M, Nle, L, or V;
J¹⁹ is E or R; and
J²¹ is V, M, or Nle.

29. The composition of matter of claim 18, wherein the PTH/PTHrP modulating domain is selected from Table 1B.

30. The composition of matter of claim 1 wherein the PTH/PTHrP modulating domain is of the formula
(SEQ ID NO: 5)
O^{N}LHO¹⁰O¹¹O¹²KSIO¹⁶O¹⁷LRRRFO²³LHHLIO^{C}
wherein:
O^{N} is absent or is YO¹O²O³O⁴O⁵O⁶O⁷, O¹O²O³O⁴O⁵O⁶O⁷, O²O³O⁴O⁵O⁶O⁷, O³O⁴O⁵O⁶O⁷, O⁴O⁵O⁶O⁷, O⁵O⁶O⁷, O⁶O⁷, or O⁷;
O¹ is an amino acid residue;
O² is an amino acid residue;
O³ is an amino acid residue;
O⁴ is an amino acid residue;
O⁵ is an amino acid residue;
O⁶ is an amino acid residue;
O⁷ is an amino acid residue;
O¹⁰ is an amino acid residue;
O¹¹ is an amino acid residue;
O¹² is an amino acid residue;
O¹⁶ is an amino acid residue;
O¹⁷ is an amino acid residue;
O²³ is an amino acid residue;
O^{C} is absent or is O²⁹, O²⁹O³⁰, O²⁹O³⁰O³¹, O²⁹O³⁰O³¹O³², O²⁹O³⁰O³¹O³²O³³, O²⁹O³⁰O³¹O³²O³³O³⁴, O²⁹O³⁰O³¹O³²O³³O³⁴O³⁵, or O²⁹O³⁰O³¹O³²O³³O³⁴O³⁵O³⁶; and
O²⁹ through O³⁶ are each independently amino acid residues;
provided that one or more of O¹⁴ through the C-terminal residue is a cysteine residue.

31. The composition of matter of claim 30, wherein:
O^{N} is O⁷;
O⁷ is a nonfunctional residue;
O¹⁰ is an acidic or hydrophilic residue;
O¹¹ is a basic or nonfunctional residue;
O¹² is an aromatic or nonfunctional residue;
O¹⁵ is a hydrophilic or nonfunctional residue;
O¹⁶ is a hydrophilic residue;
O¹⁷ is an acidic or nonfunctional residue;
O²³ is an aromatic residue; and
O^{C} is absent.

32. The composition of matter of claim 30, wherein:
O^{N} is O¹O²O³O⁴O⁵O⁶O⁷;
O¹ is a nonfunctional amino acid residue;
O² is a nonfunctional amino acid residue;
O³ is a hydrophilic amino acid residue;
O⁴ is an acidic amino acid residue;
O⁵ is a basic or nonfunctional amino acid residue;
O⁶ is a hydrophilic amino acid residue;
O⁷ is a nonfunctional residue;
O¹⁰ is an acidic or hydrophilic residue;
O¹¹ is a basic or nonfunctional residue;
O¹² is an aromatic or nonfunctional residue;
O¹⁵ is a hydrophilic or nonfunctional residue;
O¹⁶ is a hydrophilic residue; and
O¹⁷ is an acidic or nonfunctional residue; and
O²³ is an aromatic residue.

33. The composition of matter of claim 32, wherein:
O¹ is A;
O² is V;
O³ is S;
O⁴ is E;
O⁵ is H or I;
O⁶ is Q;
O⁷ is L;
O¹⁰ is N or D;
O¹¹ is K or L;
O¹² is G, F, or W;
O¹⁵ is I or S;
O¹⁶ is Q or N;
O¹⁷ is D or L;
O²³ is F or W.

34. The composition of matter of claim 30, wherein the PTH/PTHrP modulating domain comprises a sequence selected from Table 2 in which one or more residues at position 14 through the C-terminus is substituted with a cysteine residue.

35. The composition of matter of claim 1, wherein the PTH/PTHrP modulating domain has the amino acid sequence of TIP39.

36. The composition of matter of claim 1, wherein the composition of matter is selected from Table 4.

37. A composition of matter, which comprises a peptide selected from Table 1B.

38. A method of treating osteopenia, which comprises administering a PTH agonist and a bone resorption inhibitor, wherein the PTH agonist comprises a composition of matter of claim 1.

39. A method of treating osteopenia, which comprises administering a PTH agonist and a bone resorption inhibitor, wherein the PTH agonist comprises a composition of matter of claim 4.

40. A method of treating osteopenia, which comprises administering a PTH agonist and a bone resorption inhibitor, wherein the PTH agonist comprises a composition of matter of claim 18.

41. A method of treating osteopenia, which comprises administering a PTH agonist and a bone resorption inhibitor, wherein the PTH agonist comprises a composition of matter of claim 30.

42. A method of treating osteopenia, which comprises administering a PTH agonist and a bone resorption inhibitor, wherein the PTH agonist comprises a composition of matter of claim 37.

43. The method of claim 38, wherein the bone resorption inhibitor is selected from OPG, OPG-L antibody, calcitonin, bisphosphonates, estrogens, estrogen receptor modulators, and tibolone.

44. The method of claim 39, wherein the bone resorption inhibitor is selected from OPG, OPG-L antibody, calcitonin, bisphosphonates, estrogens, estrogen receptor modulators, and tibolone.

45. The method of claim 40, wherein the bone resorption inhibitor is selected from OPG, OPG-L antibody, calcitonin, bisphosphonates, estrogens, estrogen receptor modulators, and tibolone.

46. The method of claim 41, wherein the bone resorption inhibitor is selected from OPG, OPG-L antibody, calcitonin, bisphosphonates, estrogens, estrogen receptor modulators, and tibolone.

47. The method of claim 42, wherein the bone resorption inhibitor is selected from OPG, OPG-L antibody, calcitonin, bisphosphonates, estrogens, estrogen receptor modulators, and tibolone.

48. A method of treating osteopenia, which comprises administering a composition of matter of claim 1.

49. A method of treating osteopenia, which comprises administering a composition of matter of claim 4.

50. A method of treating osteopenia, which comprises administering a composition of matter of claim 18.

51. A method of treating osteopenia, which comprises administering a composition of matter of claim 30.

52. A method of treating osteopenia, which comprises administering a composition of matter of claim 37.
